(19) United States
(12) Patent Application Publication
(10) Pub. No.: US 2016/0319239 A1
(43) Pub. Date: Nov. 3, 2016

(54) COMPOSITIONS AND METHODS FOR CULTURING CELLS FROM NORMAL HUMAN TUBO-OVARIAN EPITHELIUM AND HUMAN TUBO-OVARIAN TUMORS

(71) Applicants: Whitehead Institute for Biomedical Research, Cambridge, MA (US); The Brigham and Women's Hospital, Inc., Boston, MA (US)

(72) Inventor: Tan A. Ince, Miami, FL (US)

(21) Appl. No.: 15/147,541

(22) Filed: May 5, 2016

Related U.S. Application Data
(63) Continuation of application No. 14/007,008, filed on Feb. 7, 2014, now abandoned.
(60) Provisional application No. 61/467,949, filed on Mar. 25, 2011.
(60) Provisional application No. 61/467,363, filed on Mar. 24, 2011.

(51) Int. Cl. C12N 5/00; C12N 5/071; C12N 5/09; G01N 33/50

(52) U.S. Cl. CPC C12N 5/0018; G01N 33/5011; C12N 5/0682; C12N 5/0693; C12N 2500/38; C12N 2500/16; C12N 2500/22; C12N 2500/20; C12N 2500/25; C12N 2500/32; C12N 2501/01; C12N 2501/11; C12N 2501/39; C12N 2500/46; C12N 2500/36; C12N 2501/395; C12N 2500/40

(57) ABSTRACT

Described herein are cell culture media, kits and methods for preparing cell culture media, and methods for culturing cells, for example, cells of the female reproductive tract, and tumor cells.

### CROSS-REFERENCE TO RELATED APPLICATIONS

This application claims the benefit of U.S. Provisional Application No. 61/467,363, filed on Mar. 24, 2011 and U.S. Provisional Application No. 61/467,949, filed on Mar. 25, 2011, the disclosures of which are incorporated herein by reference in their entirety.

### BACKGROUND OF THE INVENTION

Ex vivo culture of cell lines has greatly aided the study and treatment of human disease. Today, the mechanistic origins of many disorders have been characterized in cultured cell lines, and cell lines have been used both for screening and for manufacturing therapeutics. In the fields of cancer and women's reproductive health, work has been enhanced by the culture of normal cells and tumor cells from the female reproductive tract. However, many cell types prove difficult to culture using available media and existing techniques. For example, current methods for culturing normal ovarian cells are unable to distinguish between subtypes of ovarian cells, such as ciliated versus non-ciliated cells or ovarian surface epithelium versus inclusion cyst epithelium. In addition, fallopian tube epithelial cells, which have been implicated as putative cells-of-origin for high-grade papillary serous adenocarcinomas, have not been cultured ex vivo. Tumor cells derived from ovarian tumors have also proven difficult to grow in culture. While a small number of ovarian cancer cell lines exists (Verschraegen C F et al. Clin Cancer Res. 2003 February; 9(2):845-52 and U.S. Pat. No. 5,710,038), these cell lines have not been well-characterized in terms of population doubling information, clinical-pathological subtype of the original tumor tissue, verification of malignant origin, or phenotype. Accordingly, there remains a need in the art for culture cell media, kits, and methods for establishing cell lines derived from ovarian tissues, fallopian tube cells, and tumor cells.

### SUMMARY OF INVENTION

One aspect of the present invention relates to a cell culture medium, or a kit for preparing a cell culture medium, which comprises adenosine triphosphate; a carrier protein (e.g., albumin, such as bovine serum albumin); cholesterol, linoleic acid, and lipoic acid; glutathione; a nucleotide salvage pathway precursor base selected from hypoxanthine, xanthine, adenine, guanine and thymidine (preferably xanthine and/or hypoxanthine); phosphoethanolamine; selenium; transferrin; triiodothyronine; vitamin A, vitamin C, and vitamin D; Zn, Mg, and Cu; an agent that increases intracellular cAMP (preferably cholera toxin); epidermal growth factor (EGF); hydrocortisone; insulin; and serum. The cell culture medium may also comprise one or more of adenosine monophosphate, vitamin E, vitamin K3, niacin, and niacinamide.

In certain embodiments wherein the agent that increases intracellular cAMP is cholera toxin, the cell culture medium may comprise between 10 ng/mL and 70 ng/mL, preferably between 15 and 30 ng/mL, such as between 20 ng/mL and 25 ng/mL of cholera toxin. In certain preferred embodiments, the cell culture medium comprises about 20 ng/mL of cholera toxin. In other preferred embodiments, cell culture medium comprises about 25 ng/mL of cholera toxin.

In certain embodiments, the cell culture medium comprises between 3 ng/mL and 50 ng/mL of EGF, preferably about 8-12 ng/mL, such as 10 ng/mL of EGF.

In certain embodiments, the cell culture medium comprises between 0.005 μg/mL and 1.5 μg/mL of hydrocortisone and/or between 1.0 μg/mL and 75.0 μg/mL of insulin.

In certain embodiments, the cell culture medium comprises between 0.2% and 4.0% v/v of serum. In certain embodiments, the cell culture medium comprises between 0.2% and 10.0% v/v or between 0.2% and 5.0% v/v of serum.

Some embodiments of the present invention relate to a cell culture medium adapted for the culture of tumor cells (such as ovarian tumor cells) and comprising between 1.0% and 10.0% v/v of serum, between 1.0% and 4.0% v/v of serum, preferably between 1.8% v/v and 2% v/v of serum, most preferably about 1.8% v/v of serum. In some such embodiments, the cell culture medium comprises between 0.15 μg/mL and 0.3 μg/mL of hydrocortisone, preferably about 0.15 μg/mL of hydrocortisone and/or between 5.0 μg/mL and 50.0 μg/mL of insulin, preferably about 15.0 μg/mL of insulin. An exemplary medium adapted for culture of tumor cells is WIT-oc.

In such embodiments adapted for the culture of certain ovarian tumor cells, such as those derived from endometrioid tumors and mucinous tumors, the cell culture medium further comprises an estrogen, for example an estrogen (e.g., 17-beta-estradiol) at a concentration of equivalent potency of between 30 nM and 300 nM of 17-beta-estradiol, preferably about 100 nM of 17-beta-estradiol. In other embodiments, such as those adapted for the culture of certain ovarian tumor cells, such as tumor cells derived from papillary serous tumors, clear cell tumors, carcinosarcomas, and dysgerminomas, the cell culture medium is substantially free of estrogens. The exemplary medium WIT-oc may comprise estrogen or may be substantially free of estrogen, depending on the cell type that will be cultured therein.

WIT-oc and cell culture media adapted for culture of tumor cells, such as ovarian tumor cells, may support proliferation of ovarian tumor cells for at least about 14, 25, or even 35 population doublings (PD) in vitro.

In yet other embodiments, adapted for the culture of normal ovarian and fallopian tube cells, the cell culture medium comprises between 0.25% and 0.75% v/v of serum, preferably about 0.5% v/v of serum. In this cell culture medium, the concentration of hydrocortisone is preferably between 0.25 μg/mL and 0.50 μg/mL, e.g., about 0.5 μg/mL of hydrocortisone, and/or between 5.0 μg/mL and 50.0 μg/mL, preferably about 20.00 μg/mL of insulin. An exemplary medium adapted for culture of normal ovarian and fallopian tube cells, such as epithelial cells, is WIT-fo.

WIT-fo and cell culture media adapted for culture of normal ovarian and fallopian tube cells may support proliferation of ovarian cells and/or fallopian tube cells for at least about 14, 25, or even 35 population doublings (PD) in vitro. In some embodiments, ovarian cells and/or the fallopian tube cells are immortalized cells. Immortalized cells may overexpress a catalytic subunit of telomerase, for example human telomerase reverse transcriptase (hTERT). One aspect of the invention is a culture comprising a cell in which hTERT has been overexpressed, wherein overexpression of hTERT is sufficient to render the cell capable of undergoing at least 14, 25, or 35 population doublings. In certain embodiments, the culture is a substantially purified culture of cells.

One aspect of the invention relates to a substantially purified culture of ovarian cells, wherein the ovarian cells overexpress the probesets DOK5, CD47, HS6ST3, DPP6, OSBLP3; wherein the culture comprises at least 10³ cells; and wherein the cells are capable of undergoing at least 14, 25, or 35 population doublings. Similarly, one aspect relates to a substantially purified culture of fallopian tube cells, wherein the fallopian tube cells overexpress the probsets STC2, SFRP1, SLC35F3, SHMT2, TMEM164; wherein the culture comprises at least 10³ cells, and wherein the cells are capable of undergoing at least 14, 25, or 35 population doublings. Another aspect of the invention relates to kits for preparing the cell culture media described herein. In some embodiments, the kit may comprise a first one or more containers comprising adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu; and a second one or more containers comprising an agent that increases intracellular cAMP; epidermal growth factor (EGF); hydrocortisone; insulin; and serum; and optionally estrogen, such that combining the contents of the first and second containers (or first and second sets of containers) in an appropriate proportion results in the cell culture medium, e.g., as variously defined herein. In some embodiments, this kit may support proliferation of cells for at least 14, 25, or even at least 35 population doublings (PD) in vitro.

Still another aspect of the invention is a cell culture medium supplement, wherein the supplement comprises an agent that increases intracellular cAMP; epidermal growth factor (EGF); hydrocortisone; insulin; serum; and optionally, estrogen, in relative concentrations such that adding an appropriate proportion of the supplement to a basal cell culture medium results in the cell culture media described herein. For example, adding the supplement to a cell culture medium may yield 0-70 ng/mL of the agent that increases intracellular cAMP, at least 3 ng/mL of EGF, 0.015-0.5 μg/mL of hydrocortisone; at least 10.00 μg/mL of insulin, 0.2%-4.0% v/v of the serum supplement, and optionally, 30-300 nM of estrogen, e.g., as described variously herein.

A further aspect of the invention relates to methods of preparing the cell culture media described herein. In some embodiments, the method comprises combining adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu; an agent that increases intracellular cAMP; epidermal growth factor (EGF); hydrocortisone; insulin; and serum; and optionally estrogen, whereby combining the contents in an appropriate proportion results in a cell culture medium as variously described herein. In some embodiments, one or more ingredients (for example, the first eleven ingredients, adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu) are added from a first one or more containers, and one or more additional ingredients, for example, the latter five ingredients and optionally estrogen are added from a second one or more containers.

Yet another aspect of the invention is a method for culturing ovarian and fallopian tube epithelial cells. This method comprises obtaining ovarian and fallopian tube epithelial cells from an ovary or a fallopian tube; and culturing the cells in the cell culture media described herein, wherein the ovarian and fallopian tube epithelial cells can undergo at least 14, 25, or even 35 population doublings in the cell culture medium. The cells may be ovarian epithelial cells, such as those originating from an ovarian surface. The cells may be fallopian tube epithelial cells, such as those originating from the fimbriated surface of the fallopian tube.

Similarly, an aspect of the invention relates to a method for culturing tumor cells. This method comprises obtaining tumor cells; culturing the tumor cells in the cell culture media adapted for culture of tumor cells as described herein and exemplified by WIT-oc, wherein the agent increases intracellular cAMP is cholera toxin, and the cell culture medium comprises 25 ng/mL of cholera toxin, 10 ng/mL of epidermal growth factor (EGF), 0.15 μg/mL of hydrocortisone; 15.00 μg/mL of insulin; and 1.8% of the serum supplement (and preferably substantially free of estrogens), and wherein the tumor cells can undergo at least 14, 25, or even 35 population doublings in the cell culture medium. In some embodiments, the method further comprises treating culture plates with trypsin for the first 1-5 cell passages, thereby enriching for cancer cells. In other embodiments, the tumor cells are ovarian cancer cells, human breast cancer cells, pancreatic cancer cells, adenoid cystic carcinoma cells, and/or neuroendocrine tumor (carcinoid) cells from the lung or the gastrointestinal tract. For example, tumor cells may be ovarian cancer cells. The ovarian cancer cells may be obtained from primary solid ovarian tissues or ascites fluid of a patient, or from tumor xenografts grown in an animal model. In further embodiments, the tumor cells are papillary serous tumor cells, clear cell tumor cells, carcinosarcoma cells, or dysgerminoma cells. In other embodiments, e.g., for culturing endometrioid tumor cells or mucinous tumor cells, the cell culture medium may further comprise 100 nM of 17-beta-estradiol or an equipotent amount of another estrogen.

An additional aspect of the invention relates to a method of identifying candidate therapeutic agents, which comprises culturing cells (i.e., cells derived from a primary tumor, such as an ovarian tumor which is at least one of a papillary serous tumor, a clear cell tumor, a carcinosarcoma, a dysgerminoma, an endometrioid tumor, or a mucinous tumor); contacting the cells with an agent; and measuring physiology of the cells; wherein the agent that modulates the physiology of the cells is a candidate therapeutic agent. In some embodiments, the method further comprises growing the cells in a test animal, prior to contacting the cells with the agent.

### BRIEF DESCRIPTION OF DRAWINGS

FIG. 1A shows the anatomy of ovary and fallopian tubes.

FIG. 1B also shows the ovaries, the female reproductive organs in which oocytes (egg cells) are produced. The ovaries are attached to the uterus with a ligament and juxtaposed to the fimbriated end of the fallopian tubes.

FIG. 1C shows the ovarian surface, which is covered with a thin epithelium. During ovulation the oocytes burst through the ovarian surface and released into the fimbriated end of the fallopian tube. During the next 7 days the oocytes travel up the fallopian tube into the endometrial cavity of the uterus.

FIG. 1D shows the site of the ovulation is normally repaired, but sometimes small inclusion cysts that are lined with epithelium are left behind under the surface of the ovary. Figures are adapted from Baba A I, Câtoi C., Comparative Oncology. Bucharest: The Publishing House of the Romanian Academy; 2007 and the National Cancer Institute website about ovarian epithelial cancer treatment on the World Wide Web at www.cancer.gov/cancertopics/ovarianepithelial.

FIGS. 2A and 2B illustrate the results of long term culture of normal ovarian and fallopian tube epithelial cells in WIT-fo medium.

FIG. 2A shows growth of fallopian tube epithelium (FTE) cells in WIT-fo medium ( blue curve) or in Control medium (▪ red curve). As control medium a 1:1 mixture of Dulbecco's Modified Eagle's Medium (DMEM) and Ham's F12, supplemented with 5% serum was used. In WIT-fo medium FTE cells divided without a decrease in their proliferation rate continuously at least 120 days and reached 24 population doublings ( blue curve). In contrast, matched cells from the same donor growth arrested in 14 days in the control medium ((▪ red curve).

FIG. 2B shows growth of ovarian epithelium (OSE) cells in WIT-fo medium ( blue curve) or in Control medium (▪ red curve). As control medium a 1:1 mixture of MCDB 105/Medium 199 with 10% fetal bovine serum, 2 mm 1-glutamine and 10 ng/ml epidermal growth factor was used. In WIT-fo medium OSE cells divided without a decrease in their proliferation rate continuously for at least 40 days and reached 15 population doublings ( blue curve). In contrast, matched cells from the same donor growth arrested in a few weeks and never reached 3 population doublings in the control medium (▪ red curve).

FIGS. 3A-3G are a series of panels that show that PAX8, CK7 and FOXJ1 identify specific cell subtypes in normal human ovary and fallopian tube epithelium.

FIGS. 3A-3D show normal human ovarian tissue; immunoperoxidase staining of formalin-fixed paraffin embedded (FFPE) sections with PAX8. Review of numerous samples from different patients demonstrates that most of the ovarian inclusion cysts are lined by a PAX8+ epithelium (brown nuclear stain in FIG. A and FIG. B). In contrast, ovarian surface epithelium is almost always PAX8 negative (FIG. A, FIG. C), with occasional inclusion cyst epithelium that can also be PAX8 negative (FIG. D).

FIG. 3E shows normal human fallopian tube tissue; double immunoperoxidase staining of FFPE sections. The ciliated cells are FOXJ1+(nuclear brown) and non-ciliated cells are PAX8+(nuclear red).

FIG. 3F shows normal human fallopian tube tissue; double immunoperoxidase staining of FFPE sections. The ciliated cells are FOXJ1+(nuclear brown), and non-ciliated cells are Keratin 7+(cytoplasmic red). The double immunostains (FIG. E, FIG. F) were performed by sequential FOXJ1-HRP (brown) followed by PAX8/CK7 alkaline phosphatase (red) staining (scale bar=20 μM).

FIG. 3G shows that cultured cells lines exhibit immunoprofiles that are consistent with ovarian cyst epithelium (OSE) and non-ciliated fallopian tube epithelium (FNE).

FIGS. 4A-4E are a series of panels that illustrate that normal ovarian inclusion cyst (OC) and fallopian tube non-ciliated epithelium (FN) cells are immortalized with hTERT alone in WIT-fo medium.

FIG. 4A shows OC cells expressing hTERT-gfp, live cell imaging (bar=50 μM).

FIG. 4B shows FN cells expressing hTERT-gfp, live cell imaging (bar=50 μM).

FIG. 4C shows Fluorescence-Assisted Cell Sorting (FACS), in which almost all the cells in culture are gfp positive (red and blue curves) compared to parental ovarian epithelial cells (black curve).

FIG. 4D shows ovarian inclusion cyst epithelium cells that express hTERT (OCE) grown in WIT-fo medium ( blue curve) or in Control medium (MCDB 105/Medium 199 (1:1 mixture) with 10% fetal bovine serum, 2 mm 1-glutamine and 10 ng/ml epidermal growth factor) (▪ red curve). In WIT-0 medium OCE cells divided without a decrease in their proliferation rate continuously for at least 150 days and reached 70 population doublings (blue line). In contrast, matched cells from the same donor growth arrested in a few days in the control medium (▪ red curve).

FIG. 4E shows fallopian tube non-ciliated epithelium cells that express hTERT (FNE), grown in WIT-fo medium or in Control medium (1:1 mixture of Dulbecco's Modified Eagle's Medium (DMEM) and Ham's F12, supplemented with 0.1% BSA, 5% serum (▪ red curve). In WIT-fo medium FNE cells divided without a decrease in their proliferation rate continuously at least 150 days and reached 40 population doublings ( blue curve). In contrast, matched cells from the same donor growth arrested in a few days in control medium (▪ red curve).

FIGS. 5A-5C are a series of panels that show the tumorigenic transformation of normal human ovarian (OCE) and fallopian tube (FNE) cells.

FIG. 5A shows OC and FN cells that were isolated from the same donor and immortalized with hTERT alone in step 1, giving rise to OCE1 and FNE1 cells respectively. These cells were transformed into tumorigenic cells with SV40T/t (SV40ER) in step 2, and with a mutant H-Ras V12 in step 3, giving rise to tumorigenic ovarian epithelial cells (OCLER1) and tumorigenic fallopian tube epithelial cells (FNLER1). These experiments were repeated with cells from a second donor giving rise to OCLER2 and FNLER2 cells.

FIG. 5B is a western immunoblot showing the expression of SV40 LT and H-Ras proteins in OCLER1/FNLER1 pair (donor 1) and OCLER2/FNLER2 pair (donor 2), compared to negative staining in OCE and FNE cells expressing only hTERT.

FIG. 5C shows immunofluorescence images, which demonstrate similar expression of SV40-Large T Ag (LT, nuclear green) and H-Ras (Ras, cytoplasmic green) protein expression in OCLER and FNLER cells. DAPI nuclear stain; 40× magnification, scale bar=50 μM. Large T Ag (LT, nuclear green) and H-Ras (Ras, cytoplasmic green) protein expression in OCLER and FNLER cells. DAPI nuclear stain; 40× magnification, scale bar=50 μM.

FIGS. 6A-6D are a series of images that show OCLER and FNLER tumor histopathology in immunocompromised nude mice.

FIGS. 6A and 6B show hematoxylin and eosin (H&E) stains of representative tumor sections from ovarian OCLER (FIG. 6A) and fallopian tube FNLER (FIG. 6B) tumor xenografts. Both tumors were predominantly high grade and poorly differentiated, however, focally rare micropapillary structures reminiscent of papillary serous adenocarcinoma were present.

FIGS. 6C and 6D show PAX8 immunoperoxidase stains of representative FFPE tumor sections from OCLER (FIG. 6C) and FNLER (FIG. 6D) xenografts confirm the OC and FN origin of these tumors. Histological sections were prepared from formalin-fixed paraffin-embedded (FFPE) tumor tissues that were explanted 5 to 9 weeks following intraperitoneal and subcutaneous injection of cells (scale bar=20 μM).

FIGS. 7A-7E are a series of figures that collectively show that FNLER tumors are more metastatic than OCLER tumors.

FIGS. 7A and 7B show hematoxylin and eosin staining of formalin-fixed paraffin-embedded (FFPE) xenograft tumors shows FNLER (FIG. 7A) and OCLER (FIG. 7B) invading mouse pancreas. (scale bar=250 μM).

FIG. 7C shows metastases of FNLER tumors to mouse lungs from intraperitoneal or subcutaneous injection sites were initially observed as GFP positive tumor nodules on fluorescence dissecting microscope (scale bar=250 μM).

FIGS. 7D, 7E, and 7F show metastatic FNLER cells were identified in FFPE mouse lungs by H&E (FIG. 7D) as well as SV40 LT positive (FIG. 7E) and P53 positive (FIG. 7F) stained cells. (scale bar=50 μM).

FIG. 7G shows the frequency of lung metastasis, which was significantly higher for FNLER tumors as compared with OCLER tumors (P<0.05, Mann-Whitney test). Among mice with a total tumor burden >0.5 g, the frequency of metastasis to the lungs from intraperitoneal and subcutaneous injection sites was compared between OCLER and FNLER after 5-9 weeks of tumor incubation in vivo. There was no significant difference in tumor burden or time of incubation between FNLER and OCLER.

FIGS. 8A-8F collectively indicate that the gene expression signature of OCE and FNE cells identifies ovary-like and fallopian-tube like gene expression patterns in human ovarian adenocarcinoma tissues. The 10 most significant ovary/fallopian tube cell-of-origin genes were validated in two independent ovarian cancer datasets.

FIG. 8A shows association between each gene and clinical variables in the Wu dataset (8 genes were present in Wu dataset due to array platform differences). Coefficients >0 or <0 signify up- or down-regulation, respectively, of each gene with higher grade, stage or serous subtype. P-values from linear logistic regression (grade and stage as ordinal variables) were corrected for false discovery rate.

FIG. 8B is a density plot that shows a somewhat bimodal distribution of OV/FT-like scores in Wu tumor samples, supporting our segregation into OV-like and FT-like tumor clusters.

FIG. 8C shows the association of OV-like and FT-like subgroups in the Wu data with clinical characteristics (P-values from logistic regression (grade, stage as ordinal variables) and Fisher's Exact test (histological subtype).

FIG. 8D is a density plot that demonstrates a slight left skewing of OV/FT-like scores in Tothill data which suggests a small subpopulation (arrow) of OV-like tumors.

FIG. 8E shows the association of OV- and FT-like subgroups in the Tothill data with clinical features (P-values calculated as in (FIG. 8c).

FIG. 8F shows the Kaplan-Meier plots demonstrate significant differences in survival between OV- and FT-like subgroups in the Tothill data (Cox proportional hazard P-values were adjusted for tumor grade, stage and histological subtype).

FIGS. 9A and 9B collectively show that the transformed cell-like signature (TCS) and immortalized cell-like signature (ICS) identify human ovarian adenocarcinomas with different outcomes.

FIG. 9A shows the Kaplan-Meier survival curve shows that human ovarian cancers with a TSC+ expression signature (red line) have a significantly better overall survival than tumors with a ICS+ signature (blue line) (p=0.046). As expected, Limma models (including the FDR multiple testing correction) identified a large number of probes (n=17,641) that were differentially expressed at the p<0.05 level of significance. We selected the 1,000 most highly significant probes from the tumor vs. normal comparison in cell lines, and directly applied this gene signature to classify ovarian tumors in the same two independent datasets (Tothill) using consensus kmeans clustering to identify (k=2) clusters of ‘tumor-like’ and ‘normal-like’ samples. Among 136 serous high-grade (G3), advanced-stage (FIGO SIII/IV) tumors in the Tothill dataset, two clusters with high cluster stability (cluster consensus=0.89 and 0.94) were identified that included 51 and 85 samples, respectively.

FIG. 9B shows that 51 samples in the Tothill dataset were identified as expressing a “transformed cell signature” (TSC) by kmeans clustering, and 85 samples were identified as having an Immortalized Cell Signature (ICS). Out of the 51 cases we classified as TCS only 3 were classified as Tothill as poor prognosis (C1) and 46/85 ISC tumors were classified as poor prognosis (C1) by Tothill.

FIGS. 10A-10C collectively show the culture of ovarian tumors in newly developed WIT-OC media vs. conventional cell culture medium.

FIG. 10A is a chart showing the population doublings of ovarian carcinoma cells in WIT-oc. Primary ovarian papillary serous carcinoma tissue (OCI-U1a) was plated into WIT-oc ( blue curve), or MCDB-105/M199 with 10% serum (▴ green curve), or RPMI 1640 with 10% serum (▪ red curve). The cells were counted weekly, and passaged into a new flask. After 7 weeks nearly all cells in MCDB-105/M199 and RPMI-1640 media were killed (green and red lines). In contrast the cells in WIT-oc grew at a nearly constant rate and reached at least 30 population doublings, when the experiment was stopped. One clone arose in RPMI-1640 around day 90 (red line) and was established as a permanent cell line.

FIG. 10B depicts a comparative genomic hybridization (CGH) array analysis, in which the genomic DNA of the OCI-U1a tumor cells that were grown in WIT-oc and RPMI were compared to the original tumor. The whole genome CGH trace reveals several peaks that are gained (amplifications, red arrows) and lost (deletions, green arrows) in tumor cells grown in RPMI (top trace) compared to uncultured tumor sample (middle trace). In contrast, the CGH pattern of tumor cells grown in WIT-oc was significantly more similar to the uncultured tumor (bottom and middle CGH traces respectively).

FIG. 10C is a graph showing that the ovarian clear cell carcinoma cells (OCI-5Cx) were grown at least 60 population doublings in WIT-oc medium ( blue curve). In the control medium (DME:F12 with 10% serum, the cells growth arrested and died in 30-40 days (▪ red curve).

FIGS. 11A and 11B collectively show that tubo-ovarian tumor cell lines (OCI) cannot be cultured in ATCC/ECACC recommended standard ovarian tumor cell medium.

FIG. 11A shows an equal number of OCI lines (706, 29, and 4) plated in WIT-oc medium vs. McCoy's 5A, MCDB105/M199, or RPMI-1640 media supplemented with 15% serum. The cells were counted after 5 days in culture. The OCI cell lines were rapidly killed in these standard medium and were not possible to culture.

FIG. 11B shows an equal number of ATCC/ECACC ovarian cancer cell lines (SKOV-3, OV-90, and A2780) plated in WIT-oc medium vs. McCoy's 5A, MCDB105/M199, or RPMI-1640 media supplemented with 15% serum. The cells were counted after 5 days in culture. All the ATCC/ECACC ovarian cancer cell lines proliferated in WIT-oc medium as well as their recommended medium. The A2780 cells proliferated better in WIT-oc, than the recommended standard medium.

FIGS. 12A-12F is a series of images illustrating the histopathology of OCI xenografts, which recapitulate the original tumor. Formalin fixed paraffin embedded primary tumor and xenograft tumor tissue sections were stained with Hematoxylin and eosin.

FIG. 12A shows a tumor xenograft section of ECACC ovarian cancer cell line SKOV3.

FIG. 12B shows a tumor xenograft section of ATCC ovarian cancer cell line OV90. There is no specific architecture of the xenograft tumors derived from ATCC lines (FIGS. 12A-B). The typical features of human adenocarcinomas such as glands, papillae, stromal cores, mucin or hormone receptor expression, desmoplastic stroma are absent. These tumors are almost entirely composed of sheets of tumor cells, and appearance that is similar to cells in tissue culture.

FIG. 12C shows a tumor xenograft section of OCI-P9a line derived from a human papillary serous adenocarcinoma (PSC). Note the clear presence of stromal cores and papillary architecture.

FIG. 12D shows a tumor xenograft section of OCI-E1p line derived from a human endometrioid adenocarcinoma. Note the clear presence of glands. Inset: immunohistochemistry confirms estrogen receptor and mucin expression in these xenograft tumors consistent with the endometrioid phenotype.

FIG. 12E shows an original tumor section of the human PSC tumor tissue from which OCI-P9a was derived from. Note stromal cores and papillary architecture.

FIG. 12F shows an original tumor section of the human endometrioid adenocarcinoma tissue from which OCI-E1p line derived from.

FIG. 13 shows a comparison of OCI lines vs. original tumor tissue with SNP array analysis. The genomic DNA from 12 primary uncultured tumors (T, black) and 12 continuous tubo-ovarian cell lines derived from them in WIT-oc medium (OCI, red), and nine ATCC/ECACC lines (blue) were hybridized to a 250 Affymetrix SNP array and the results were analyzed with Genechip. Blue=copy number alteration or loss of heterozygosity, yellow=no detectable DNA alteration. Note that in this unsupervised hierarchical clustering analysis every OCI cell line is clustered next to its parental uncultured primary tumor sample. Hence, each cell line is correctly identified as being most similar to its parental original tumor tissue. The vast majority of the alterations in the OCI lines involved significantly shorter segments of the chromosomes compared to ATCC/ECACC ovarian cancer cell lines. While OCI-U1p and OCI-P3a had large segment alterations, this appears to be a feature of the original tumors they were derived from and not a consequence of cell culture artifact (TU1 and TP3 respectively).

FIG. 14 illustrates that gene expression profile of OCI lines recapitulate clinical subgroups of human ovarian adenocarcinomas in culture. The unsupervised hierarchical clustering of mRNA expression levels of OCI lines, normal ovary-fallopian tube cells and ATCC/ECACC ovarian cancer lines were measured with Affymetrix U133Plus chips (red=high expression, blue=low expression). Note that normal ovary and fallopian tube cells (blue bar), OCI lines derived from papillary serous (red bar) and clear cell/endometrioid (green) tumors formed distinct clusters. The ATCC/ECACC ovarian cancer cell lines formed a distinct cluster (black) separate from the other OCI lines with the exception of several clear cell OCI lines (pink).

FIG. 15 shows that the proteomic profile of OCI lines recapitulates clinical subgroups of human ovarian adenocarcinomas in culture. The Reverse Phase Protein Assay (RPPA) analysis of OCI lines were carried out, examining the expression of >200 proteins. RPPA is a high-throughput and quantitative proteomic technology that allows quantitative analysis of the differential expression of hundreds of proteins simultaneously. These assays are carried out by printing serial dilution of protein extracts from cells in array on a slide or membrane and probe the array with a single antibody. The specific binding of the antibody to each spot on the array is quantitated and plotted. The RPPA analysis of OCI lines revealed that the cell lines that are established from different tumor types formed separate clusters, similar to the mRNA expression profiles. These results suggest that the cultured cells must retained significant level of cell biological information in culture that allows their molecular features recognized as normal ovarian and fallopian cells (blue bar), clear cell tumors (green bar), endometrioid tumors (pink bar) and papillary serous tumors (red bar). A few of the OCI lines did not cluster with their own histotype; which may reflect heretofore unrecognized molecular subtypes or variants of these major histotypes. Since there was only one example mucinous carcinoma, carcinosarcoma and mullerian carcinoma (yellow) in this data set, the ability of these cell types to cluster according tumor origin could not be assessed. These experiments were repeated in triplicate with similar results.

FIGS. 16A-C collectively show a significantly different drug response in OCI lines as compared to ATCC/ECACC lines.

FIG. 16A shows the ATCC/ECACC ovarian tumor lines (SKOV3, OV90, TOV-1120 and A2780) and the OCI ovarian tumor lines (C5×, P9a1, P7a and FCI-P1p) plated in WIT-oc medium (5000 cells/well) in 96 well plates in triplicate. The next day serial dilutions of MAPK inhibitor UO126 were added to the plate. The number of viable cells was measured as 590/530 florescence with an Alamar Blue assay after 144 hrs with drug incubation. There was a significant difference in the effect of the MAPK inhibitor on OCI cell lines vs. ATCC/ECACC cell lines. The concentration of UO126 that reduced the cell numbers by 50% (LD₅₀) in OCI was approximately 5 uM for most ATCC/ECACC cells as expected. However, surprisingly most of the OCI lines were at least 5-6 times more resistant to MAPK inhibition (LD₅₀>30 uM).

FIG. 16B shows the OCI ovarian tumor lines (P7a, P2a, C2p) and ATCC/ECACC ovarian tumor lines (ES2 and OV90) plated in WIT-oc medium (5000 cells/well) in plates in triplicate. The next day serial dilutions of paclitaxel were added to the plate. The viable cells were measured as 590/530 florescence with an Alamar Blue assay after 72 hrs with drug incubation.

FIG. 16C shows the OCI ovarian tumor lines (P7a, P2a, C2p) and ATCC/ECACC ovarian tumor lines (ES2 and OV90) plated in WIT-oc medium (5000 cells/well) in plates in triplicate. The next day serial dilutions of cisplatin were added to the plate. The viable cells were measured as 590/530 florescence with an Alamar Blue assay after 72 hrs with drug incubation. There was a dramatic difference in the effect of the paclitaxel or cisplatin on OCI cell lines vs. ATCC/ECACC cell lines. The concentration of these drugs that produced a 50% decrease in tumor cell numbers (LD₉₀) was more than 6-fold higher in OCI lines compared to ATCCC/ECACC lines.

FIGS. 17A and 17B collectively show a comparison of OCI and ATCC/ECACC ovarian tumor cell line gene expression with human ovarian tumors.

FIG. 17A shows hierarchical clustering of gene expression data of 37 cells and 285 human tissues. Genes with an expression level that has at least 2-fold difference relative to median value across tissues in at least 4 cells were selected for hierarchical clustering analysis (3,831 gene features). The data are presented in matrix format in which rows represent individual gene and columns represent each tissue. Each cell in the matrix represents the expression level of a gene feature in an individual tissue. The red and green color in cells reflect relative high and low expression levels respectively as indicated in the scale bar (log 2 transformed scale). Purple bar=human tumor samples, red bar OCI tumor lines, blue bar ATCC ovarian tumor lines+ OCI lines that co cluster with ATCC lines.

FIG. 17B shows clinical overall survival analysis data of human ovarian tumors in panel a. The human tumors that have a gene expression profile that is similar to OCI lines have a worse outcome than human tumors that have gene expression profile similar to ATCC lines.

FIGS. 18A-18F are images of several other tumor types that have been successfully grown in WIT-oc medium.

FIGS. 18A-18B are microscopic images of the tumor cell lines that have been established from two different samples of human invasive ductal carcinoma of the breast.

FIGS. 18C-18D are microscopic images of the tumor cell lines that have been established from two different samples of head and neck tumor Adenoid Cystic Carcinoma.

FIGS. 18E-18F are microscopic images of the tumor cell lines that have been established from two different samples of human neuroendocrine tumor (carcinoid) of the lung.

### DETAILED DESCRIPTION

### I. Cell Culture Medium

A. Composition of Culture Medium

The subject invention relates to cell culture media that support long-term, growth and proliferation of primary cells (including ovarian and fallopian tube epithelial cells) and tumor cells (including ovarian tumor cells) in vitro. The phrases “cell culture medium,” “culture medium” (plural “media” in each case) and “medium formulation” refer to a nutritive solution for cultivating cells and may be used interchangeably.

Cells cultured in the media described herein can grow for at least 4 weeks (or 15 population doublings), up to several months, in such a culture medium without losing differentiation potential. In one embodiment, the subject medium supports long-term growth and proliferation of ovarian and fallopian epithelial cells, and/or ovarian and fallopian tube cells that have been transfected with telomerase, for at least about 15 weeks or at least about 35 population doubling (PD) in vitro, without any additional detectable genetic alterations, or losing differentiation potential. In another embodiment, the subject medium supports growth and proliferation of these cells for at least 5, 6, 7, 8, 9, 10, 11, 12, 13, 14, 15 weeks or more in culture. Similarly, the subject medium may support growth of primary cell lines derived from human tumor tissue, from tumor cells in malignant body fluids, and/or primary xenografts of tumors grown in animal models for at least about 15 weeks or at least about 14, 25 or even 35 population doubling (PD) in vitro, without any additional detectable genetic alterations, or losing its phenotype (morphological, structural, and/or molecular, etc.) and/or histopathology. In another embodiment, the subject medium supports growth and proliferation of these cells for at least 5, 6, 7, 8, 9, 10, 11, 12, 13, 14, 15 weeks or more in culture.

The cell culture media of the present invention are aqueous-based (but can be reconstituted from dry powder and/or frozen components), comprising a number of ingredients in water, liquid, and/or an aqueous solution.

The term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

By “cell culture” or “culture” is meant the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

In some embodiments, the medium is substantially free of one or more specified components. In certain embodiments, “substantially free” refers to a low amount of the component that has no statistically significant effect on cell growth and/or differentiation state. In some embodiments, “substantially free” refers to less than 1%, 0.1%, 0.01%, 0.001%, or 0.0001% v/v of a liquid or w/v of a solute. In some embodiments, “substantially free” refers to a concentration of less than 0.001, 0.0001, 0.00001, 0.000001, or 0.0000001 mg/L. In some embodiments, “substantially free” refers to a concentration of less than 10 nM, 1 nM, 100 pM, 10 pM, or 1 pM.

In one aspect of the invention, the subject cell culture medium comprises: (1) one or more antioxidants; (2) one or more nucleotide salvage pathway synthesis precursors; (3) one or more lipid synthesis precursors; (4) one or more protein synthesis precursors; (5) one or more carbohydrate synthesis and energy metabolism precursors; (6) one or more buffers (not essential); (7) one or more cations (monovalent and/or divalent), ions, trace metals and enzyme cofactors; (8) one or more carrier proteins (such as bovine serum albumin); (9) one or more detergents (such as tween80); (10) one or more agents that induce increased intracellular 3′-5′ cyclic adenosine monophosphate (cAMP) levels; (11) one or more hormones and growth factors, and/or (12) serum. In other embodiments, one or more of the above-listed categories of components may be omitted, provided that the resulting medium supports growth and/or proliferation of cells such as normal ovarian and fallopian tube cells and/or tumor cells such as ovarian tumor cells for at least about 4 weeks or at least about 15, 25, or even 35 population doublings (PD) in vitro.

Thus, the media of the invention comprise one or more antioxidants; nucleotide synthesis and salvage pathway precursors; lipid synthesis precursors; agonists of intracellular cAMP level; hormones and growth factors; and serum. The media may additionally comprise other components such as amino acid supplements, vitamins necessary for cell growth/proliferation, trace minerals, inorganic salts, energy sources (e.g. for glycolysis), and other components such as pH indicators, etc. In other words, ingredients of the present invention may include amino acids, vitamins, inorganic salts, adenine, D-glucose, N-[2-hydroxyethyl]piperazine-N′-[2-ethanesulfonic acid] (HEPES), hydrocortisone, insulin, lipoic acid, phenol red, phosphoethanolamine, putrescine, sodium pyruvate, triiodothyronine (T3), thymidine and transferrin. Each of these ingredients may be obtained commercially, for example from Sigma (Saint Louis, Mo.).

While not wishing to be bound by any particular theory, antioxidants generally help to quench free-radicals, which are thought to be detrimental to cell growth in general. The antioxidants of the invention may include, without limitation, one or more of the following: beta-carotene, vitamin E, vitamin C (ascorbic acid), vitamin K3, glutathione (reduced), niacin (or niacinamide), or DTT (dithiothreitol). The antioxidants may optionally be supplemented with trace metals, including Zn, Se, Cr, Cu, Mg, or Mn.

Again, without wishing to be bound by theory, trace minerals may be necessary for the constitution of certain enzymes. For example, glutathione peroxidase uses selenium and glutathione superoxide uses copper as a cofactor. It was postulated that in diseases where there is a large free radical load, there may be deficiencies of these trace elements in a particular microenvironment. The presence of trace minerals may be helpful to enzymatic antioxidants (which may have been devoid of the cofactors). Thus the presence of the trace minerals may allow effective use of the enzymatic antioxidants by the host. Since it is known that zinc can up-regulate superoxide dismutase and selenium can up-regulate glutathione peroxidase, increasing trace minerals in a given microenvironment would produce a net increase in enzymatic antioxidants in the microenvironment. A net increase in the enzymatic antioxidants and increasing amphipathic antioxidant would further reduce oxidative damage to tissue or cells, as well as other deleterious effects due to free radicals.

Thus many inorganic salt ingredients, cations, ions, trace metals, and vitamins, which may be beneficial in the media of the present invention include a calcium salt (e.g., CaCl₂), CuSO₄, FeSO₄, KCl, a magnesium salt (e.g., MgCl₂), Sodium acetate, NaCl, NaHCO₃, Na₂HPO₄, Na₂SO₄ and ions of the trace elements selenium, and zinc. Optionally, additional inorganic salt ingredients may include a manganese salt (e.g., MnCl₂), silicon, molybdenum, vanadium, nickel, and tin.

These trace elements may be provided in a variety of forms, preferably in the form of salts such as Na₂SeO₃, and ZnSO (or Na₂SiO₃, (NH₄)₆MO₇O₂₄, NH₄VO₃, NiSO₄, SnCl₂ for optional salts). These inorganic salts and trace elements may be obtained commercially, for example from Sigma (Saint Louis, Mo.).

Vitamin ingredients which may be included in the media of the present invention include biotin, choline chloride, D-Ca⁺⁺-pantothenate, folic acid, i-inositol, niacinamide, pyridoxine, riboflavin, thiamine and vitamins A and B12. These vitamins may be obtained commercially, for example from Sigma (Saint Louis, Mo.).

Protein synthesis procursors include amino acid ingredients. In one embodiment, the amino acid ingredients which may be included in the media of the present invention include L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. These amino acids may be obtained commercially, for example from Sigma (Saint Louis, Mo.).

Alternatively, in some other embodiments, only essential amino acids are included in the media of the present invention. Certain cells, such as human cells must have adequate amounts of 9 amino acids to survive. These so called “essential” amino acids cannot be synthesized from other precursors. However, cysteine can partially meet the need for methionine (they both contain sulfur), and tyrosine can partially substitute for phenylalanine. Such essential amino acids include: Histidine, Isoleucine, Leucine, Lysine, Methionine (and/or cysteine), Phenylalanine (and/or tyrosine), Threonine, Tryptophan, and Valine. In certain embodiments, only Histidine, Isoleucine, Leucine, Lysine, Threonine, Tryptophan, and Valine are included.

Some or all of the above ingredients, when admixed together in solution, can form a “basal medium.” To this basal medium, other components, such as at least one nucleotide synthesis and/or salvage pathway precursors (e.g. hypoxanthine), epidermal growth factor (EGF), at least one agent increasing intracellular cyclic adenosine monophosphate (cAMP) levels, and at least one hormone, and at least one protein, are added to formulate the complete culture media of the present invention. These latter added components, such as EGF and the cAMP-increasing agent(s) may be added to freshly formulated basal medium, or they may be admixed as in a stock solution stored frozen, preferably at about −20° C. to about −70° C., until being added to basal medium to formulate the complete medium of the present invention. This complete medium may comprise BPE or other organ/gland extracts in animal cell culture media to achieve the desired cell growth and proliferation, or may be substantially free of BPE or other organ/gland extracts in animal cell culture media. The admixture may also be prepared as a 1-1000× formulation, most preferably as a 1×, 100×, 500× or 1000× formulation, which is then diluted appropriately into culture medium to provide a 1×final formulation in the complete media of the present invention.

The media of the invention may also include one or more hormones, such as: progesterone, testosterone, hydrocortisone, or estrogen, and/or one or more growth factors such as: insulin and EGF (epidermal growth factor).

For example, the media of the invention may comprise EGF, which may be natural or recombinant, and may be human or rodent. EGF available commercially (e.g., from GIBCO/LTI, Gaithersburg, Md.), isolated from natural sources or produced by recombinant DNA techniques (U.S. Pat. No. 4,743,679) according to methodologies that are routine in the art. To formulate the medium of the present invention, in preferred embodiments EGF is added to a medium (such as a medium shown in Table 3) to reach a final concentration of about 0.00001-10 mg/L, preferably about 0.0005-1 mg/L.

The media of the invention may also include nucleotide analogs or precursors, such as hypoxanthine, xanthine, adenine, guanine, and thymidine that can be used in the salvage pathway synthesis of nucleotides.

The media of the invention may also include lipid synthesis precursors, such as: cholesterol, linoleic acid, lipoic acid, or O-phosphoryl ethanolamine.

The medium of the invention also includes one or more cAMP agonists or agents that increase intracellular cAMP levels. A variety of such agents may be used in formulating the media of the present invention. Included are agents which induce a direct increase in intracellular cAMP levels (e.g., dibutyryl cAMP), agents which cause an increase in intracellular cAMP levels by an interaction with a cellular G-protein (e.g., cholera toxin and forskolin), agents which cause an increase in intracellular cAMP levels by acting as agonists of B-adrenergic receptors (e.g., isoproterenol) and agents which cause an increase in intracellular cAMP levels by inhibiting the activities of cAMP phosphodiesterases (e.g., isobutylmethylxanthine (IBMX) and theophylline). Most preferable for use in formulating the media of the present invention is cholera toxin. These cAMP-increasing agents are available commercially, e.g. from Sigma (St. Louis, Mo.), and are used at concentrations approximating those described in Green (Proc. Natl. Acad. Sci. USA 15:801-811 (1978)). For example, cholera toxin is added to the basal medium described above at a concentration of about 0-0.01 mg/L, preferably about 0-0.001 mg/L, and most preferably about 0-0.0001 mg/L. Dibutyryl cAMP, IBMX, isoproterenol etc. can be added to achieve the same level of cAMP as cholera toxin.

It is also desirable to increase intracellular cAMP level by using agents such as cholera toxin, forskolin, G protein-coupled receptor agonists, PKC agonists. In addition, cells may have increased cAMP in response to the beta-adrenergic agonist isoproterenol (Iso), prostaglandin E(2) (PGE(2)), certain prostanoid receptor-selective agonists (beraprost, butaprost), and an adenosine receptor agonist. In addition, overexpression of AC type 6 or agents inhibiting cyclic nucleotide phosphodiesterases increased cellular cAMP levels.

The subject medium may also comprise one or more carbohydrate synthesis and energy metabolism precursors, such as D-glucose, sodium pyruvate, etc.

The subject medium may also comprise one or more carrier proteins, such as bovine serum albumin (BSA). Carrier protein may be a protein which transports specific substances through the cell membrane in which it is embedded and into the cell. Different carrier proteins may be required to transport different substances, as each one is designed to recognize only one substance, or group of similar substances. Certain carrier proteins may bind to one or more media components (such as growth factors, etc.) and confer them extra stability in the media, or to facilitate certain biological processes (e.g. acyl-carrier protein, sterol carrier protein, hormone carrier protein, etc.).

The subject medium may also comprise one or more surfactants, such as nonionic surfactants Tween 60 or Tween 80. Again, without wishing to be bound by any particular theory, such detergent components may help to wet, solubilize, emulsify, or disperse certain media components. For example, they may prevent aggregation of proteins such as BSA, increase solubility of certain components, and may even enhance the function of certain enzymes.

Although not considered essential, the subject medium may additionally comprise one or more buffer systems, such as HEPES and sodium bicarbonate buffer systems, such that a balanced pH is maintained in long-term culture. Frequent, constant or continuous change of culture medium may also help to restore medium pH in fast growing cells.

To illustrate, Table 3 below shows exemplary compositions of three media formulation of the instant invention that supports long-term growth and proliferation of (1) ovarian tumor cell lines derived from papillary serous tumor, clear cell tumor, carcinosarcoma, or dysgerminoma, (2) ovarian tumor cell lines derived from endometrioid tumor or mucinous tumors, and (3) normal ovarian or fallopian tube cells. These media support growth and proliferation of these cell types for at least about 4 weeks or at least about 15 population doubling (PD) in vitro, with a phenotype (morphological, structural, and/or molecular, etc) and/or histopathology that is indistinguishable from the original tumor from which the cells were derived.

The percentage of fibroblasts and other stromal cells decreases sharply after a few passages and population doublings, to the extent that no appreciable amount of fibroblast and stromal cell differentiation markers (e.g. vimentin) can be detected. For example, the epithelial differentiation markers may include keratin 8, keratin 10, keratin 14, keratin 18, keratin 19, E-cadherin, p63, SMA (smooth muscle actin), and β-catenin.

Ex vivo tissue culture exposes cells to oxidative damage, metabolic stress and DNA damage that induce p53 and p16 genes, which in turn induce cell-cycle arrest, senescence and/or apotosis limiting the life-span cultured cells. In another embodiment, the media of the invention supports long-term stress-free growth and proliferation of primary cells. One indication of such stress-free growth in the subject medium is indicated by the low/undetectable expression level of CDK inhibitor p16 and tumor suppressor p53. These two proteins are typically induced to express at a high level in stressed cells, but not healthy growing cells in tissue culture media. In this embodiment the cells can be grown at 37° C., 5% CO₂ and varying O₂ concentrations, e.g., 1%, 2%, 3%, or ambient air.

In another embodiment, the media of the invention is substantially free of at least one member selected from the group consisting of: heparin, fibroblast growth factor (FGF), and bovine pituitary extract (BPE). In certain embodiments, none of the above listed components are present in the subject medium.

However, to the extent that such components do not substantially affect the performance of the medium in terms of supporting primary cell growth and proliferation, the subject medium may in certain embodiments include and tolerate the presence of one or more of such components.

The invention provides embodiments of the media of Table 3 in which any one or more of the components having a concentration range with a lower limit of 0 are absent and embodiments in which such component is present. It should be understood that the medium of the invention as listed in Table 3 is merely for illustrative purposes only. Although the medium itself is sufficient for certain intended purposes, especially culturing the cell types associated herein with each formulation, not all components listed in these Tables may be necessary or even optimum for their intended purposes. A skilled artisan, partly depending on the need for the specific primary cells in question, could readily determine if any listed component is necessary and/or optimum by, for example, eliminating one component or changing the concentration of one component at a time and comparing the growth/proliferation of specific type of cultured cells in such a modified medium with the original medium. One or more components may also be substituted by other chemicals of similar properties when necessary. Such modified medium without one or more non-essential/unnecessary components are within the scope of the invention. Similarly, a skilled artisan could also determine the optimal level of any given component for a particular cell type, by, for example, testing a range of concentrations (e.g., 10%, 25%, 50%, 75%, 100%, 2-, 5-, 10-, 20-, 50-, 100-, 200-, 500-, 1000-fold higher, or 10%, 25%, 50%, 75%, 100%, 2-, 5-, 10-, 20-, 50-, 100-, 200-, 500-, 1000-fold lower) for each listed component based on or starting from the listed concentration of that particular component. Some components have a listed range of concentrations. The proper or optimal concentration for any particular cell types can also be determined similarly starting from the listed concentration. In doing such tests, initial broad-range concentration tests may be narrowed down later based on the outcomes of the initial experiments. For example, for an initial test, the concentration of one component of interest may be changed to 10⁻³, 10⁻², 10⁻¹, 10-fold, 100-fold, and 1000-fold of a concentration listed in Table 3. If the 10⁻² test still supports the desired growth, while 10⁻³ fails to, then the 10-fold concentration difference between 10⁻² and 10⁻³ may be further explored in the second round of test to pin-point the best ranges. Thus, media so optimized for specific cell types are also within the scope of the instant invention.

As will be readily apparent to one of ordinary skill in the art, the concentration of a given ingredient can be increased or decreased beyond the range disclosed and the effect of the increased or decreased concentration can be determined using only routine experimentation. The optimization of the present media formulations for any specific cell type can be carried out using approaches described by Ham (Ham, Methods for Preparation of Media, Supplements and Substrata for Serum-Free Animal Culture, Alan R. Liss, Inc., New York, pp. 3-21, 1984) and Waymouth (Waymouth, C., Methods for Preparation of Media, Supplements and Substrata for Serum-Free Animal Culture, Alan R. Liss, Inc., New York, pp. 23-68, 1984). The optimal final concentrations for medium ingredients are typically identified either by empirical studies, in single component titration studies, or by interpretation of historical and current scientific literature. In single component titration studies, using animal cells, the concentration of a single medium component is varied while all other constituents and variables are kept constant and the effect of the single component on viability, growth or continued health of the animal cells is measured.

It will be understood that certain vitamins and hormones listed herein can exist in different forms, as known in the art (e.g., different naturally occurring or non-naturally occurring forms), and can be used as substitutes for one another. It will also be appreciated that where the instant application discloses a vitamin or hormone, the invention should be understood to encompass embodiments in which any form of such vitamin or hormone having similar biological activity (or compound(s) that can be modified or metabolized in cell culture medium or intracellularly to provide a biologically active form) is used in the inventive media and/or method(s).

It will be appreciated that compounds such as estrogen, progesterone, thyroid hormone, hydrocortisone, insulin, etc., can be substituted in whole or in part by other compounds (naturally occurring or non-naturally occurring, isolated from natural sources or at least in part chemically synthesized) that are agonists of the estrogen receptor, progesterone receptor, thyroid hormone receptor, glucocorticoid receptor, insulin receptor, respectively. A number of such compounds are known in the art.

The medium ingredients can be dissolved in a liquid carrier or maintained in dry form. If dissolved in a liquid carrier at the preferred concentrations shown above (i.e., a “1×formulation”), the pH of the medium should be adjusted to about 7.0-7.6, preferably about 7.1-7.5, and most preferably about 7.2-7.4. The osmolarity of the medium should also be adjusted to about 275-350 mOsm, preferably about 285-325 mOsm, and most preferably about 280-310 mOsm. The type of liquid carrier and the method used to dissolve the ingredients into solution vary and can be determined by one of ordinary skill in the art with no more than routine experimentation. Typically, the medium ingredients can be added in any order.

A cell culture medium is composed of a number of ingredients and these ingredients vary from one culture medium to another. A “1×formulation” is meant to refer to any aqueous solution that contains some or all ingredients found in a cell culture medium at working concentrations. The “1×formulation” can refer to, for example, the cell culture medium or to any subgroup of ingredients for that medium. The concentration of an ingredient in a 1×solution is about the same as the concentration of that ingredient found in a cell culture formulation used for maintaining or cultivating cells in vitro. A cell culture medium used for the in vitro cultivation of cells is a 1×formulation by definition. When a number of ingredients are present, each ingredient in a 1×formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic aced. A “1×formulation” of these amino acids contains about the same concentrations of these ingredients in solution. Thus, when referring to a “1×formulation,” it is intended that each ingredient in solution has the same or about the same concentration as that found in the cell culture medium being described. The concentrations of ingredients in a 1×formulation of cell culture medium are well known to those of ordinary skill in the art. See Methods For Preparation of Media, Supplements and Substrate For Serum-Free Animal Cell Culture Allen R. Liss, N.Y. (1984), which is incorporated by reference herein in its entirety. The osmolarity and/or pH, however, may differ in a 1×formulation compared to the culture medium, particularly when fewer ingredients are contained in the 1×formulation.

A “10×formulation” is meant to refer to a solution wherein each ingredient in that solution is about 10 times more concentrated than the same ingredient in the cell culture medium. For example, a 10×formulation of RPMI-1640 culture medium may contain, among other ingredients, 2.0 g/L L-arginine, 0.5 g/L L-asparagine, and 0.2 g/L L-aspartic acid (compare 1×formulation, above). A “10×formulation” may contain a number of additional ingredients at a concentration about 10 times that found in the 1×culture medium. As will be readily apparent, “25×formulation,” “50×formulation,” “100×formulation,” “500×formulation,” and “1000×formulation” designate solutions that contain ingredients at about 25-, 50-, 100-, 500-, or 1000-fold concentrations, respectively, as compared to a 1×cell culture medium. Again, the osmolarity and pH of the media formulation and concentrated solution may vary.

Preferably, the solutions comprising ingredients are more concentrated than the concentration of the same ingredients in a 1×media formulation. The ingredients can be 10-fold more concentrated (10×formulation), 25-fold more concentrated (25×formulation), 50-fold more concentrated (50×concentration), or 100-fold more concentrated (100×formulation). More highly concentrated formulations can be made, provided that the ingredients remain soluble and stable. See U.S. Pat. No. 5,474,931 (entire contents incorporated herein by reference), which is directed to methods of solubilizing culture media components at high concentrations.

If the media ingredients are prepared as separate concentrated solutions, an appropriate (sufficient) amount of each concentrate is combined with a diluent to produce a 1×medium formulation. Typically, the diluent used is water but other solutions including aqueous buffers, aqueous saline solution, or other aqueous solutions may be used according to the invention.

The culture media of the present invention are typically sterilized to prevent unwanted contamination. Sterilization may be accomplished, for example, by filtration through a low protein-binding membrane filter of about 0.1-1.0 μm pore size (available commercially, for example, from Millipore, Bedford, Mass.) after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1×sterile medium formulation. Autoclaving or other elevated temperature-based methods of sterilization are not favored, since many of the components of the present culture media are heat labile and will be irreversibly degraded by temperatures such as those achieved during most heat sterilization methods.

As will be readily apparent to one of ordinary skill in the art, each of the components of the culture medium may react with one or more other components in the solution. Thus, the present invention encompasses the formulations disclosed in Table 3, supplemented as described above, as well as any reaction mixture which forms after these ingredients are combined.

Many tissue culture media typically contain one or more antibiotics, which are not necessary for cell growth/proliferation per se, but are present to inhibit the growth of undesirable microbes, such as bacteria and/or fungi.

Antibiotics are natural chemical substances of relatively low molecular weight produced by various species of microorganisms, such as bacteria (including Bacillus species), actinomycetes (including Streptomyces) and fungi, that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics are: (1) the β-lactams, including the penicillins, cephalosporins and monobactams; (2) the aminoglycosides, e.g., gentamicin, tobramycin, netilmycin, and amikacin; (3) the tetracyclines; (4) the sulfonamides and trimethoprim; (5) the fluoroquinolones, e.g., ciprofloxacin, norfloxacin, and ofloxacin; (6) vancomycin; (7) the macrolides, which include for example, erythromycin, azithromycin, and clarithromycin; and (8) other antibiotics, e.g., the polymyxins, chloramphenicol and the lincosamides.

Antibiotics accomplish their anti-bacterial effect through several mechanisms of action which can be generally grouped as follows: (1) agents acting on the bacterial cell wall such as bacitracin, the cephalosporins, cycloserine, fosfomycin, the penicillins, ristocetin, and vancomycin; (2) agents affecting the cell membrane or exerting a detergent effect, such as colistin, novobiocin and polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; (4) agents affecting nucleic acid metabolism, e.g., the fluoroquinolones, actinomycin, ethambutol, 5-fluorocytosine, griseofulvin, rifamycins; and (5) drugs affecting intermediary metabolism, such as the sulfonamides, trimethoprim, and the tuberculostatic agents isoniazid and para-aminosalicylic acid. Some agents may have more than one primary mechanism of action, especially at high concentrations. In addition, secondary changes in the structure or metabolism of the bacterial cell often occur after the primary effect of the antimicrobial drug.

Thus for convenience and other practical reasons, the subject media may be additionally supplemented by one or more antibiotics or other substances that inhibit the growth/proliferation of undesirable bacteria/fungi/virus. In other embodiments, however, the subject medium may be free of any antibiotics to ensure optimum growth of primary cells. Extra care should be taken when handling cells growing in antibiotic-free medium in order to avoid possible contamination.

The concentrations listed in Table 3 are not absolute and invariable. Since different cell types may have different growth needs, it is contemplated that generally, a 2-10 fold variation (increase or decrease) for each value is an acceptable range of concentration. Some components may tolerate an even larger variation of final concentration. Further optimization can be achieved using these starting concentrations.

In some embodiments the final concentration of any one or more of the added components differs from that listed in Table 3 by a factor of up to 10, by which is meant that the relevant concentration may range from 0.1 to 10 times that listed in Table 3. In some embodiments the final concentration of any one or more of the added components differs from that listed in Table 3 by a factor of up to 3, by which is meant that the relevant concentration may range from 0.3 to 3 times that listed in Table 3. In some embodiments the final concentration of any one or more of the added components differs from that listed in Table 3 by a factor of up to 2, by which is meant that the relevant concentration may range from 0.5 to 2 times that listed in Table 3. In some embodiments the added concentration of any one or more of the added components differs from those listed in Table 3 by up to 10%, 20%, or 50% from the value listed in Table 3, respectively.

The invention encompasses embodiments in which any 1, 2, 3, 4, or 5 component(s) is/are not added to the medium. In some embodiments the medium is supplemented with insulin, EGF, hydrocortisone, cholera toxin, and serum. Optionally the medium is further supplemented with estrogen.

B. Culture of Normal Ovarian and Fallopian Tube Cells

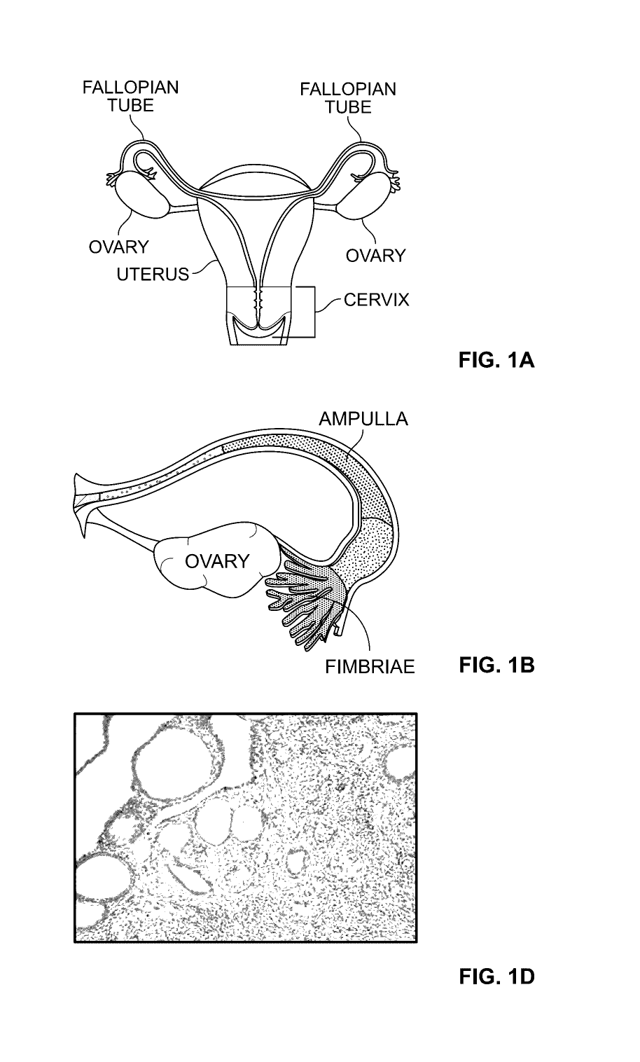

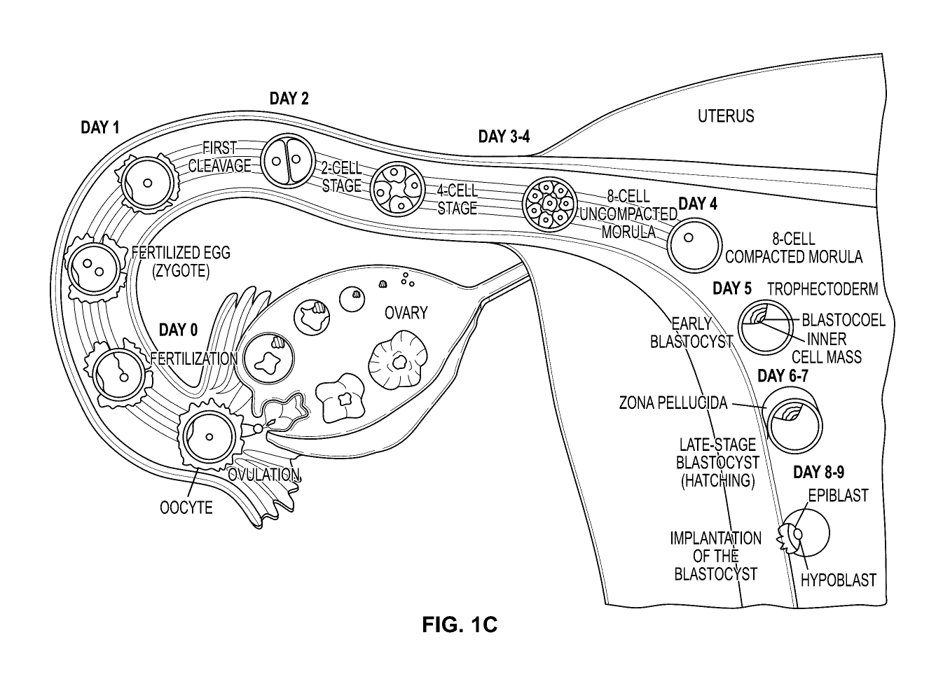

The ovaries are female reproductive organs that are composed of oocytes (egg cells), stromal cells that support the oocytes and epithelium that covers the surface ovary. During ovulation the oocytes (egg cells) burst through the ovarian surface and are captured by the fimbriated end of the fallopian tube. These oocytes migrate up the fallopian tube into the endometrial cavity where they implant (FIG. 1 A-C).

The epithelium of internal organs confines a central lumen such as the milk duct, bowel lumen, bronchial lumen etc. The functional distinction of epithelial cells it that they either secrete substances into the lumen or absorb substances from the lumen; such as secretion of saliva, breast milk, lung mucus, gastric acid, pancreatic enzymes, or absorption of water and nutrient by the gastrointestinal tract and kidney epithelium. The entire tissue structure that is surrounding the lumen is sometimes referred to as a ‘duct’ or ‘gland’. The common functions that are shared by these cells give them unique properties that make them distinct group different from all other cells in the body.

The surfaces of the ovaries are covered with a single layer epithelium, under which the oocytes reside in a zone that is sometimes referred to as the ‘ovarian cortex’. The oocytes are supported by hormonally active ovarian stromal cells that make up the bulk of the ovary. During each cycle several oocytes are released into the fallopian tubes and the gap that forms at the surface of the ovary, which is normally repaired by the ovarian surface epithelium. However, in some cases small cysts are formed at the site of ovulation, which are lined by an epithelium that is distinct from the surface epithelium. These cysts in the ovarian cortex are referred to as “inclusion cysts” (FIG. 1 D). The lumen of the fallopian tube is lined with a single layer epithelium composed of two cell types that are morphologically distinguished by the presence or absence of cilia at their apical surface.

Tumors arise from the cells in the ovaries and fallopian tube. In particular, the tumors that arise from and/or mimic the glandular and ductal epithelium of the internal organs are collectively referred to as adenocarcinomas. The term cancer or carcinoma is used by pathologists to refer to tumors that have an epithelial phenotype. Accordingly, the terms “ovarian cancer,” “ovarian carcinoma” and “ovarian adenocarcinoma” may be used interchangeably.

Notably, the vast majority of human tumors, including tubo-ovarian tumors, as well as lung, breast, colon, prostate, gastrointestinal, endocrine organ, and gynecologic tumors are epithelial adenocarcinomas. These tumors collectively account for more than ninety percent of human deaths due to cancer. The tumors that arise from all the other cells such as blood, immune, muscle, bone, neural, endothelial, and fibroblasts are very rare compared to epithelial cancers.

The tubo-ovarian adenocarcinomas that arise from the epithelium of the ovary and fallopian tube account for nearly ninety percent of malignant ovarian tumors that cause the majority of ovarian cancer related deaths. The tumors that arise from stromal and germ cells in the ovary are rare, accounting for less than five percent of deaths due to ovarian tumors.

Adenocarcinoma of the ovary is a heterogeneous disease that is comprised of at least six major histopathological subtypes with distinct cellular, morphological, and clinical features. The major subtypes of ovarian adenocarcinoma include papillary serous, mucinous, endometrioid, clear cell, squamous and transitional types, that account for more than ninety percent of ovarian adenocarcinomas.

Which cells are putative precursor lesions of tubo-ovarian tumors? The identification and detection of early non-invasive precursor lesions such as ductal carcinoma in situ in breast (DCIS), adenomatous polyps in colon, squamous intraepithelial lesion (SIL) in cervix, or endometrial intraepithelial neoplasia (EIN) has greatly helped in understanding the pathogenesis of cancers arising in these organs. Identification of putative cells-of-origin and early precursor lesions allowed development of early detection tools and a better understanding of the natural progression of tumors in these organs. Since most ovarian cancers become symptomatic very late in their progression it has not been possible to study their precursors lesions adequately. Unfortunately, there are no routine screening tools for early detection of this cancer at this time.

There have been three types of normal cells-of-origin proposed for the origin of human ovarian adenocarcinoma; historically it has been thought that ovarian cancer arises from the ovarian surface epithelium (OSE) or epithelial-lined inclusion cysts (OC) within the ovarian cortex. However, few putative precancerous lesions in the ovary have been identified and whether changes such as ovarian intraepithelial neoplasia observed in normal ovary truly represent a pre-malignant neoplastic lesion is difficult to determine. More recent studies have implicated the fallopian tube epithelium (FTE), specifically the fimbriated end, as a putative cell-of-origin for as many as half of high-grade papillary serous adenocarcinomas. This is supported by data that tubal precursor lesions and tumor tissue from the same women shared common p53 mutations.

Thus, one aspect of the invention comprises a cell culture medium that supports growth and/or proliferation of normal ovarian and fallopian tube cells, for example ovarian surface epithelium (OSE), epithelial-lined inclusion cysts (OC), and fallopian tube epithelium (FTE), for at least about 4 weeks or at least about 15, 25, or even 35 population doublings (PD) in vitro.

Epithelial cells such as the normal ovarian and/or fallopian tube cells (OSE, OC, and/or FTE) may be immortalized in order to extend the life span of the cells. Cells may be immortalized via oncogenic transformation, for example, by exposing normal cells to chemical mutagens, radiation, or other carcinogenic agents, and/or transforming normal cells by expressing viral oncogenes (e.g., H-Ras, Human Papilloma Virus E6/7 (HPV E6/7), Simian Virus 40 Large T/small t (SV40T/t) antigen, and/or adenoviral proteins (E1A)). Cells may also be immortalized by overexpressing a catalytic subunit of telomerase such as human telomerase reverse transcriptase (hTERT). In some embodiments, ovarian and/or fallopian tube cells overexpressing hTERT are cultured in a cell culture medium adapted for growth and/or proliferation of normal ovarian and fallopian tube cells. The combination of hTERT overexpression and cell culture media described herein may be sufficient to immortalize the ovarian and/or fallopian tube cells.

In some embodiments, a culture comprises a cell in which hTERT has been overexpressed, wherein overexpression of hTERT is sufficient to render the cell capable of undergoing at least 14, 25, or 35 population doublings. The culture may be a substantially purified culture. The culture may comprise at least 10³, at least 10⁴, at least 10⁵, or at least 10⁶ cells. hTERT overexpression may be due to, for example, transfection or transformation with a virus.

Ovarian cells and fallopian tube cells, such as immortalized cells, may be transformed into tumorigenic cells. Transformed tumorigenic ovarian and/or fallopian tube cells may be cultured in media adapted for growth and/or proliferation of normal ovarian and fallopian tube cells, such as described in the present section, and exemplified by WIT-fo (Table 3) in the present disclosure.

After oncogenic transformation of non-ciliated fallopian tube cells or ovarian inclusion cyst cells, these cell types are associated with expression of probes. In certain embodiments, transformed non-ciliated fallopian tube cells overexpress the probesets DOK5, CD47, HS6ST3, DPP6, OSBLP3, and transformed ovarian inclusion cyst cells overexpress the probesets STC2, SFRP1, SLC35F3, SHMT2, TMEM164.

One aspect of the invention relates to a culture of ovarian cells and/or fallopian tube cells, wherein the ovarian cells overexpress the probesets DOK5, CD47, HS6ST3, DPP6, OSBLP3, and wherein the fallopian tube cells overexpress the probesets STC2, SFRP1, SLC35F3, SHMT2, TMEM164, and wherein the culture comprises at least 10³ cells, and wherein the cells are capable of undergoing at least 14, 25, or 35 population doublings. In some embodiments, the culture comprises at least 10⁴, 10⁵, 10⁶, 10⁷, 10⁸, 10⁹, and/or 10¹⁰ cells.

In certain embodiments of any of the foregoing, a substantially purified culture of cells is a culture in which at least 50%, at least 55%, at least 60%, at least 65%, at least 70%, at least 75%, at least 80%, at least 85%, at least 90%, at least 95%, at least 98%, or greater than 98% of the cells in the culture are of a particular cell type of interest.

Accordingly, the invention provides a cell culture medium, as described above, useful for culturing normal ovarian epithelial cells and normal fallopian tube cells. In some embodiments, the cell culture medium useful for culturing normal ovarian epithelial cells and normal fallopian tube cells comprises adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu; an agent that increases intracellular cAMP; epidermal growth factor (EGF); hydrocortisone; insulin; and serum. The culture medium may further comprise at least one adenosine monophosphate and vitamin E. The culture medium may comprise at least one of vitamin K3, niacin, or niacinamide. The carrier protein in the culture medium may be albumin. The nucleotide salvage pathway precursor base in the culture medium may be xanthine and/or hypoxanthine.

The agent that increases intracellular cAMP may be cholera toxin. In some embodiments, the cell culture medium comprises cholera toxin in a concentration ranging from 1-100 ng/mL; 1-75 ng/mL; 1-50 ng/mL; 1-25 ng/mL; 1-15 ng/mL; 1-10 ng/mL; 10-100 ng/mL; 10-75 ng/mL; 10-50 ng/mL; 10-25 ng/mL; 10-15 ng/mL; 15-100 ng/mL; 15-75 ng/mL; 15-50 ng/mL; 15-25 ng/mL; 15-20 ng/mL; 20-100 ng/mL; 20-75 ng/mL; 20-25 ng/mL; 25-100 ng/mL; 25-75 ng/mL; or 25-50 ng/mL. Preferably, a cell culture medium may comprise about 20 ng/mL of cholera toxin, or alternately, about 25 ng/mL of cholera toxin.

In some embodiments, the cell culture medium comprises EGF. The concentration of EGF may range from 0.5-50 ng/mL; 0.5-25 ng/mL; 0.5-15 ng/mL; 0.5-10 ng/mL; 0.5-5.0 ng/mL; 0.5-1.0 ng/mL; 1.0-50 ng/mL; 1.0-25 ng/mL; 1.0-15 ng/mL; 1.0-10 ng/mL; 1.0-5 ng/mL; 5.0-50 ng/mL; 5.0-25 ng/mL; 5.0-15 ng/mL; 5.0-10 ng/mL; 10-50 ng/mL; 10-25 ng/mL; or 10-15 ng/mL. Preferably, a cell culture medium may comprise between 3 ng/mL and 50 ng/mL of EGF, or most preferably, may comprise about 10 ng/mL of EGF.

In some embodiments, the cell culture medium comprises hydrocortisone. The concentration of hydrocortisone may range from 0.01-5.0 μg/mL; 0.01-2.5 μg/mL; 0.01-1.0 μg/mL; 0.01-0.5 μg/mL; 0.01-0.30 μg/mL; 0.01-0.25 μg/mL; 0.01-0.10 μg/mL; 0.01-0.05 μg/mL; 0.05-5.0 μg/mL; 0.05-2.5 μg/mL; 0.05-1.0 μg/mL; 0.05-0.5 μg/mL; 0.05-0.30 μg/mL; 0.05-0.25 μg/mL; 0.05-0.10 μg/mL; 0.05-0.05 μg/mL; 0.15-5.0 μg/mL; 0.15-2.5 μg/mL; 0.15-1.0 μg/mL; 0.15-0.5 μg/mL; 0.15-0.30 μg/mL; 0.15-0.25 μg/mL; 0.15-0.10 μg/mL; and 0.15-0.05 μg/mL. Preferably, the concentration of hydrocortisone in the cell culture medium is between 0.015-5.0 μg/mL. For culture of normal ovarian and fallopian tube cells, the cell culture medium preferably comprises between 0.25 μg/mL and 0.50 μg/mL of hydrocortisone, and most preferably comprises about 0.5 μg/mL of hydrocortisone.

In some embodiments, the cell culture medium comprises insulin. The concentration of insulin may range from 1.0-75.0 μg/mL; 1.0-50.0 μg/mL; 1.0-25.0 μg/mL; 1.0-10.0 μg/mL; 10.0-75.0 μg/mL; 10.0-50.0 μg/mL; 10.0-25.0 μg/mL; and 10.0-15.0 μg/mL; 15.0-75.0 μg/mL; 15.0-50.0 μg/mL; 15.0-25.0 μg/mL; and 15.0-20.0 μg/mL. In some preferred embodiments, the range of insulin is 15.0-20.0 μg/mL. For culture of normal ovarian and fallopian tube cells, the cell culture medium preferably comprises between 5.0 μg/mL and 50.0 μg/mL of insulin. Most preferably, the cell culture medium comprises about 20.00 μg/mL of insulin.

In some embodiments, the cell culture medium comprises serum, at a concentration ranging from 0.2%-4.0% v/v; 0.5-3.0%; 1.0%-2.0% v/v; 1.5%-2.0% v/v. In some preferred embodiments for culture of normal ovarian and fallopian tube cells, the cell culture comprises between 0.25% and 0.75% v/v of serum. In most preferred embodiments, the cell culture medium comprises about 0.5% v/v of serum. In some embodiments a kit or medium does not include serum, and the serum (if present) may be supplied separately or by the user.

In some embodiments, the cell culture medium supports proliferation of ovarian cells and/or fallopian tube cells for at least about 10 population doublings (PD) in vitro. The cell culture medium may also support proliferation of ovarian cells and/or fallopian tube cells for at least about 6, 8, 10, 12, 14, 16, 18, 20, 25, 30, 35, 40, 45, or 50 PDs. Population doublings may reflect information that is distinct from the number of “subcultures” or “passages” are used as a measure of cell culture success. The subculture or passage of cells simply refers to removing cells from one culture plate and seeding them into a new culture plate. This indicates that the cells have tolerated the transfer from one plate to another, and remained alive during the transfer. However, the number of cell passage does not necessarily indicate cell proliferation.

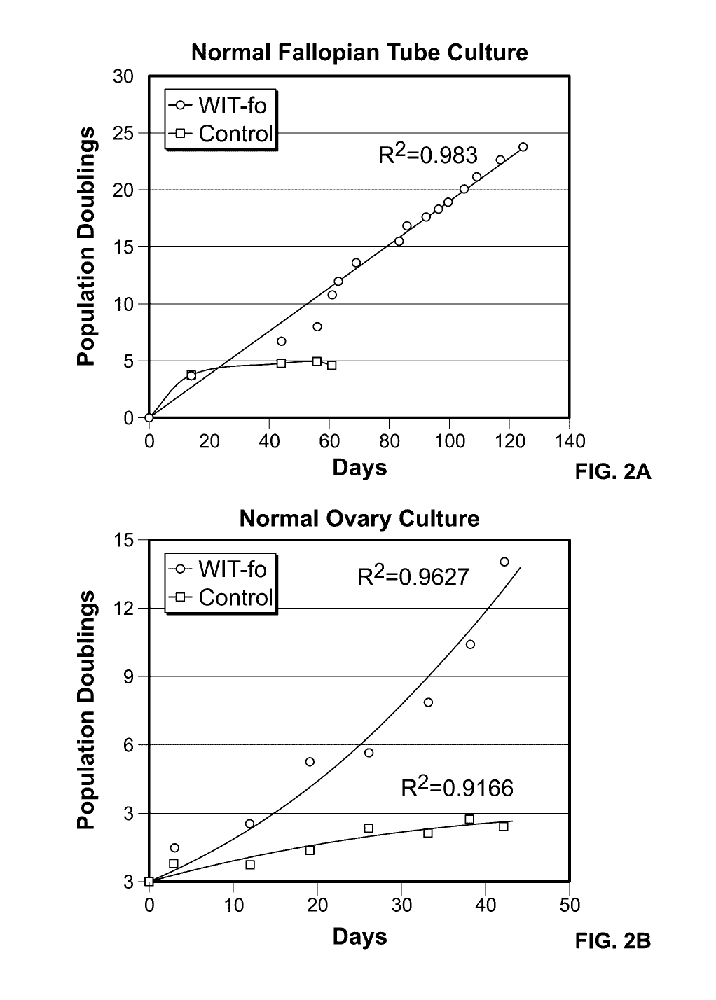

FIG. 2 illustrates the lack of correlation between cell proliferation and passage numbers. In standard control culture conditions ‘passage’ fallopian tube epithelium was passaged for nearly 60 days, during 4 passages (FIG. 2B, red curve). However, the cell growth curve remained almost flat after 14 days and there was no net increase in the number of cells. Thus, passage numbers are uninformative regarding net increase in cell numbers.

The net increase in cell numbers is most accurately measured in ‘population doublings’ (PD). A single cell will produce 1024 cells in 10 population doublings (2¹⁰=2, 4, 8, 16, 32, 64, 128, 256, 512, 1024). Hence, each 10 population doublings is approximately equal to 3 orders-of-magnitude (1,000 fold) net increase in cell numbers, 20 population doublings would be close to 1 million fold increase, and 30 population doublings would be close to 1 billion fold increase in net cell numbers. Thus, a PD vs. time chart is a semi-log 2 exponential plot. In contrast, cell passages may be equal to almost no net increase in cell numbers.

One exemplary cell culture medium is described in Table 3, in the column labeled WIT-fo. In certain embodiments, any specific ingredient is replaced at least in part by an alternate ingredient capable of fulfilling the same function. In certain embodiments of the invention, any specific ingredient listed in Table 3 is replaced at least in part by an alternate ingredient capable of fulfilling the same function. Such substitutions may be made with respect to any one or more listed ingredients (e.g., 1, 2, 3, 4, 5, 6, 7, 8, 9, 10, or more of the ingredients may be substituted by alternate ingredients capable of fulfilling the same function). In one embodiment, the buffer component is substituted. In another embodiment, the detergent or surfactant component is substituted. In another embodiment the carrier protein component is substituted. The specification provides non-limiting examples of suitable substitutes.

Another aspect of the invention relates to methods for culturing normal ovarian epithelial cells and fallopian tube cells. In one embodiment, the method for culturing ovarian and fallopian tube epithelial cells, comprises obtaining ovarian and fallopian tube epithelial cells from an ovarian surface and/or a fimbriated surface of the fallopian tube and culturing the cells in the cell culture medium as described above and in Table 3 (column labeled WIT-fo), wherein the ovarian and fallopian tube epithelial cells undergo at least 14 population doublings in the cell culture medium.

C. Culture of Tumor Cells

One aspect of the invention relates to a cell culture medium that supports primary cell lines derived from (1) solid human tumor tissue, (2) tumor cells in malignant body fluids and (3) tumor cells from primary xenografts of tumors grown in mice. The cultured cells grow substantially as phenocopies of the original tumor cell in tissue and provide clinically relevant model to discover and develop reagents for early detection, diagnosis, prognosis and treatment of human tumors.

The vast majority of human tumors, including tubo-ovarian tumors, as well as lung, breast, colon, prostate, gastrointestinal, endocrine organ, and gynecologic tumors are epithelial adenocarcinomas. These tumors collectively account for more than ninety percent of human deaths due to cancer. The tumors that arise from all the other cells such as blood, immune, muscle, bone, neural, endothelial, and fibroblasts are very rare compared to epithelial cancers.

The tubo-ovarian adenocarcinomas that arise from the epithelium of the ovary and fallopian tube account for nearly ninety percent of malignant ovarian tumors that cause the majority of ovarian cancer related deaths. The tumors that arise from stromal and germ cells in the ovary are rare, accounting for less than five percent of deaths due to ovarian tumors.

Adenocarcinoma of the ovary is a heterogeneous disease that is comprised of at least six major histopathological subtypes with distinct cellular, morphological, and clinical features. The major subtypes of ovarian adenocarcinoma include papillary serous, mucinous, endometrioid, clear cell, squamous and transitional types, that account for more than ninety percent of ovarian adenocarcinomas.

Human tumors are complex tissues that are composed of malignant tumor cells as well as dozens of different normal cell types such as epithelial cells, fibroblasts, endothelial cells, macrophages, smooth muscle cells. Moreover, many tumors are composed of both non-invasive (in situ) and invasive malignant components, as well as premalignant hyperplastic tissue that are present side by side with invasive tumor cells. Thus, when cultures are initiated from tumor tissues, many cell types that are from the different components of the tumor have an opportunity to proliferate in the culture plate. While it seems paradoxical, it is not unusual for normal stromal or normal epithelial cells, as well as the cells from the non-invasive (in situ) component of the tumor or premalignant hyperplastic cells to grow faster than the invasive tumor cells in conventional culture conditions. Thus, additional experiments that verify the malignant nature of the cells that are proliferating in the plate are needed to confirm successful culture of the tumor cells.

The soft agar colony assay may be used to verify that the cultured cells are indeed malignant tumor cells. Normal and premalignant cells are incapable of forming anchorage-independent colonies in soft agar. Thus, the ability to form colonies in this assay is an indication of the malignant nature of the cultured cells. Another method used to verify the malignant nature of the cultured cells is the in vivo tumor forming ability.

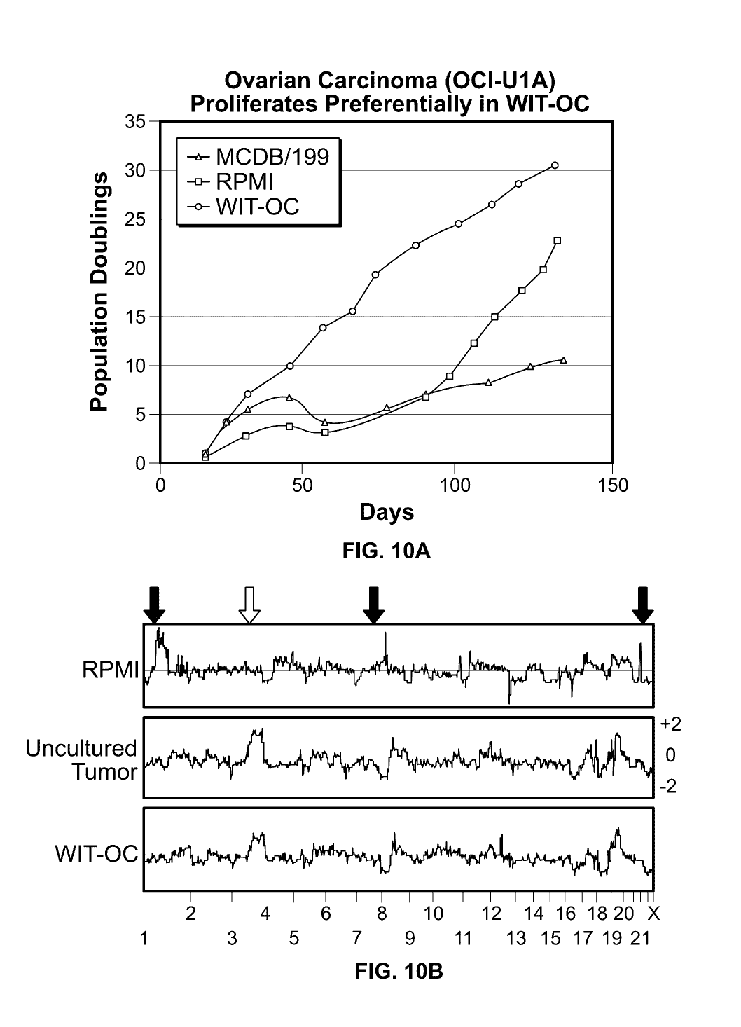

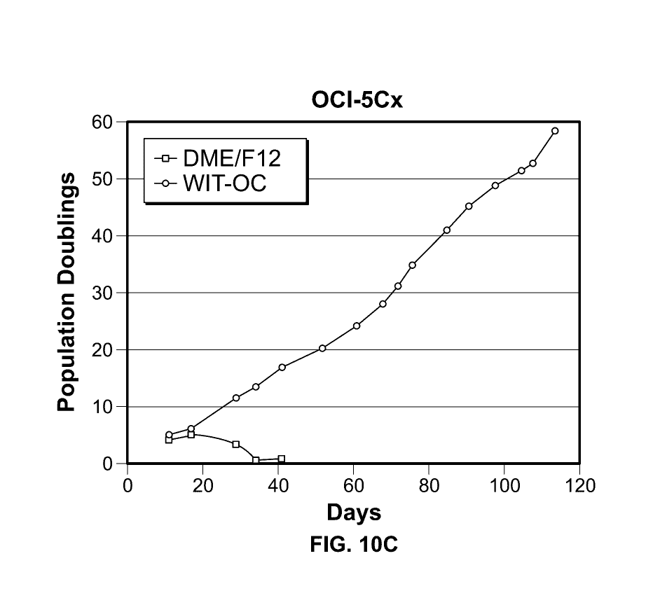

The success of tumor cell culture is expressed in terms of “passage numbers” which is at best an uninformative metric of cell proliferation. The passage of cultured cells refers to mechanically or enzymatically dissociating the cells from the surface of the culture dish or flask and transferring the cells into a new empty flask, at the point when the cells become crowded or slow to proliferate. Typically, the success of tumor cell culture is expressed in terms of “passage numbers” which can be an incomplete metric of cell proliferation. FIG. 10 shows that passage numbers do not always correlate with an increase in cell numbers. Tumor cells often slow down and stop proliferating after 2-3 passages. While the cells remain alive and can be passaged additional times, their numbers may stay the same or may even decrease. Thus, without actual cell counts, the passage numbers alone do not confirm net increase in cell numbers.

A more accurate and objective measure of cell proliferation is the total number of ‘population doublings’ (PD) achieved during continuous culture of cells. A single cell can produce 1024 cells in ten population doublings (2¹⁰=2, 4, 8, 16, 32, 64, 128, 256, 512, 1024). Hence, each ten population doublings is approximately equal to three orders-of-magnitude (1,000 fold) net increase in cell numbers, twenty population doublings would be close to one million fold increase, and thirty population doublings would be close to one billion fold increase in net cell numbers. Thus, a PD vs. time chart is a semi-log 2 exponential plot.

Thus, one aspect of the invention comprises a cell culture medium that supports growth and/or proliferation of cancer cells, for example, ovarian cancer adenocarcinoma cell lines deposited at ATCC (American Type Tissue Collection) or ECACC (the European Collection of Cell Cultures), or, cells derived from primary solid human ovarian tissues, cells derived from ascites fluid, and/or cells derived from human tumor xenografts that have been grown in mice. The cell culture medium supports growth for at least about 4 weeks and/or at least about 30 population doublings (PD) in vitro.

As the vast majority of human tumors, including tubo-ovarian tumors, as well as lung, breast, colon, prostate, gastrointestinal, endocrine organ, and gynecologic tumors are epithelial adenocarcinomas, the culture media described herein may also be used to culture tumor cells derived from human breast cancers, pancreatic cancer, adenoid cystic carcinoma, neuroendocrine tumor (carcinoid) from the lung or from the gastrointestinal tract, and other tumors related to epithelial cells.

In one embodiment, the cell culture medium for culturing tubo-ovarian tumors and cells derived therefrom comprises adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu; an agent that increases intracellular cAMP; epidermal growth factor (EGF); hydrocortisone; insulin; serum; and optionally, estrogen. The culture medium may further comprise at least one adenosine monophosphate and vitamin E. The culture medium may comprise at least one of vitamin K3, niacin, or niacinamide. The carrier protein in the culture medium may be albumin. The nucleotide salvage pathway precursor base in the culture medium may be xanthine and/or hypoxanthine.

The agent that increases intracellular cAMP may be cholera toxin. In some embodiments, the cell culture medium comprises cholera toxin in a concentration ranging from 1-100 ng/mL; 1-75 ng/mL; 1-50 ng/mL; 1-25 ng/mL; 1-15 ng/mL; 1-10 ng/mL; 10-100 ng/mL; 10-75 ng/mL; 10-50 ng/mL; 10-25 ng/mL; 10-15 ng/mL; 15-100 ng/mL; 15-75 ng/mL; 15-50 ng/mL; 15-25 ng/mL; 15-20 ng/mL; 20-100 ng/mL; 20-75 ng/mL; 20-25 ng/mL; 25-100 ng/mL; 25-75 ng/mL; or 25-50 ng/mL. Preferably, a cell culture medium may comprise about 20 ng/mL of cholera toxin, or alternately, about 25 ng/mL of cholera toxin.

In some embodiments, the cell culture medium comprises EGF. The concentration of EGF may range from 0.5-50 ng/mL; 0.5-25 ng/mL; 0.5-15 ng/mL; 0.5-10 ng/mL; 0.5-5.0 ng/mL; 0.5-1.0 ng/mL; 1.0-50 ng/mL; 1.0-25 ng/mL; 1.0-15 ng/mL; 1.0-10 ng/mL; 1.0-5 ng/mL; 5.0-50 ng/mL; 5.0-25 ng/mL; 5.0-15 ng/mL; 5.0-10 ng/mL; 10-50 ng/mL; 10-25 ng/mL; or 10-15 ng/mL. Preferably, a cell culture medium may comprise between 3 ng/mL and 50 ng/mL of EGF, or most preferably, may comprise about 10 ng/mL of EGF.

In some embodiments, the cell culture medium comprises hydrocortisone. The concentration of hydrocortisone may range from 0.01-5.0 μg/mL; 0.01-2.5 μg/mL; 0.01-1.0 μg/mL; 0.01-0.5 μg/mL; 0.01-0.30 μg/mL; 0.01-0.25 μg/mL; 0.01-0.10 μg/mL; 0.01-0.05 μg/mL; 0.05-5.0 μg/mL; 0.05-2.5 μg/mL; 0.05-1.0 μg/mL; 0.05-0.5 μg/mL; 0.05-0.30 μg/mL; 0.05-0.25 μg/mL; 0.05-0.10 μg/mL; 0.05-0.05 μg/mL; 0.15-5.0 μg/mL; 0.15-2.5 μg/mL; 0.15-1.0 μg/mL; 0.15-0.5 μg/mL; 0.15-0.30 μg/mL; 0.15-0.25 μg/mL; 0.15-0.10 μg/mL; and 0.15-0.05 μg/mL. Preferably, the concentration of hydrocortisone in the cell culture medium is between 0.015-5.0 μg/mL. For culture of tubo-ovarian tumor cells, the cell culture medium preferably comprises between 0.15 μg/mL and 0.30 μg/mL of hydrocortisone, and most preferably comprises about 0.15 μg/mL of hydrocortisone.

In some embodiments, the cell culture medium comprises insulin. The concentration of insulin may range from 1.0-75.0 μg/mL; 1.0-50.0 μg/mL; 1.0-25.0 μg/mL; 1.0-10.0 μg/mL; 10.0-75.0 μg/mL; 10.0-50.0 μg/mL; 10.0-25.0 μg/mL; and 10.0-15.0 μg/mL; 15.0-75.0 μg/mL; 15.0-50.0 μg/mL; 15.0-25.0 μg/mL; and 15.0-20.0 μg/mL. In some preferred embodiments, the range of insulin is 15.0-20.0 μg/mL. For culture of tubo-ovarian tumor cells, the cell culture medium preferably comprises between 5.0 μg/mL and 50.0 μg/mL of insulin. Most preferably, the cell culture medium comprises about 15.00 μg/mL of insulin.

In some embodiments, the cell culture medium comprises serum, at a concentration ranging from 0.2%-4.0% v/v; 0.5-3.0%; 1.0%-2.0% v/v; or 1.5%-2.0% v/v. In certain embodiments, the serum concentration ranges from 0.2%-10% v/v; 0.2%-5.0% v/v; or 1.0%-5.0% v/v. In some embodiments, the serum concentration ranges from 4.0%-10.0% v/v; 4.0-6.0% v/v; 5.0-7.0% v/v; 6.0-8.0% v/v; 7.0-9.0% v/v; or 8.0-10.0% v/v. In some preferred embodiments for culture of tubo-ovarian tumor cells, the cell culture comprises between 1.0% and 1.5% v/v of serum. In most preferred embodiments, the cell culture medium comprises about 1.2% v/v of serum.

In some embodiments, tumor cells do not require estrogen for growth. Cell culture medium as described herein that does not contain estrogen may support growth of papillary serous tumors, clear cell tumors, carcinosarcomas, and dysgerminomas.

In other embodiments, certain types of tumor cells may require estrogen for growth. For example, cell culture medium may comprise estrogen, at a concentration ranging from 30-300 nM; 30-200 nM; 30-100 nM; 65-300 nM; 65-200 nM; 65-100 nM; 100-300 nM; 100-150 nM; and 100-200 nM. For culturing endometrioid tumors and/or mucinous tumors, the cell culture medium preferably additionally comprises an estrogen in a concentration range of about 65-150 nM, and most preferably comprises an estrogen in a concentration of about 100 nM. The estrogen may be 17-beta-estradiol or its bioequivalent. A bioequivalent of 17-beta-estradiol will be present in an amount to have the same activity as 17-beta-estradiol at the concentration ranges listed above; for example, the concentration of a bioequivalent may be selected to have the same activity as 100 nM of 17-beta-estradiol.

In some embodiments, the cell culture medium supports proliferation of ovarian cells and/or fallopian tube cells for at least about 30 population doublings (PD) in vitro. The cell culture medium may also support proliferation of ovarian cells and/or fallopian tube cells for at least about 6, 8, 10, 12, 14, 16, 18, 20, 25, 30, 35, 40, 45, or 50 PDs.

Another aspect of the invention relates to methods for culturing tubo-ovarian tumor cells. In one embodiment, the method for culturing ovarian and fallopian tube epithelial cells, comprises obtaining tubo-ovarian tumor cells from a solid tumor, from ascites fluid, or from tumor cells from primary xenografts of tumors grown in mice, and culturing the cells in the cell culture medium as described above and in Table 3 (columns labeled WIT-oc), wherein the ovarian and fallopian tube epithelial cells undergo at least 30 population doublings in the cell culture medium. Some cell lines can be established from malignant ascites fluids, not continuous cell lines from the solid tumors that could be passaged more than seven times. The malignant fluid samples such as peritoneal ascites and lung effusions are free of fibroblasts and other stromal cells. Ascites is typically present in a small subset of patients in very late stages of the disease, and more common in patients with recurrent disease who have been already treated with chemotherapy. Thus, the tumor cells that are isolated from ascites are not from untreated patients. These post-treatment tumors cells may have been exposed to genotoxic drugs, hence are different from the original tumor cells.

In some embodiments, the CO₂ and O₂ levels may be modified to support culture of certain cell types. Most cell culture is carried out in ambient air (18% O₂) supplemented with carbon dioxide (CO₂) to 5%. In other embodiments, endometrioid and mucinous tumors may be cultured in WIT-oc medium supplemented with estrogen under growth conditions comprising low oxygen (O₂) adjusted to 5% and (5%) CO₂.

### II. Methods and Kits for Preparing Cell Culture Medium

Other aspects of the invention relates to methods and kits for preparing the cell culture media described herein. Kits may be used to prepare the cell culture media. For example, components may be prepared as stock solutions of one or more components and divided among containers. In some embodiments, a kit for preparing cell culture medium comprises a first container comprising adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu; and a second container comprising an agent that increases intracellular cAMP; epidermal growth factor (EGF); hydrocortisone; insulin; serum, and optionally, estrogen. The cell culture medium in such a kit may support proliferation of cells for at least 20 population doublings (PD) in vitro.

In some embodiments, a cell culture medium supplement may be added to a cell culture basal medium, in order to produce the cell culture media described herein. A supplement may comprise an agent that increases intracellular cAMP; epidermal growth factor (EGF); hydrocortisone; insulin; serum; and optionally, an estrogen. In a preferred embodiment, adding the supplement to a cell culture medium yields 0-70 ng/mL of the agent that increases intracellular cAMP, at least 3 ng/mL of EGF, 0.015-0.5 μg/mL of hydrocortisone; at least 10.00 μg/mL of insulin, 0.2%-4.0% v/v of the serum supplement, and optionally, 30-300 nM of estrogen.

Another aspect of the invention relates to a method of preparing the cell culture media described herein, comprising combining adenosine triphosphate; a carrier protein; cholesterol, linoleic acid, and lipoic acid; glutathione; a nucleotide salvage pathway precursor base selected from hypoxanthine, xanthine, adenine, guanine and thymidine; phosphoethanolamine; selenium; transferrin; triiodothyronine; vitamin A, vitamin C, and vitamin D; Zn, Mg, and Cu; an agent that increases intracellular cAMP; epidermal growth factor (EGF); hydrocortisone; insulin; serum, and optionally, estrogen. As noted above, components may be added from a first and second container, and/or from concentrated stock solutions of one or more components. In some embodiments, components can be packaged in any number of containers in any combination. In some embodiments, 2, 3, 4 or 5 containers, or between 5 and 10 containers, are used.

### III. Selective Growth, Identification and Use of Cultured Cells

A. Selective Growth of Cultured Cells

One aspect of the invention relates to the use of cell culture media to grow selective cell types from a heterogeneous population. The initial collection of ovarian tissues and/or fallopian tube tissues, for example by brushing the surface of these tissues or by biopsy, may yield more cell types than desired. Similarly, an ovarian tumor sample from a patient may contain a variety of cell types, including non-cancerous cells such as stromal cells or fibroblasts. Because the cell culture media described herein is adapted for growth of normal ovarian cells, normal fallopian tube cells, and/or tumor cells, the media will not select for other cell types. Accordingly, the cell culture media adapted for growth of normal ovarian cells may be used to culture select cell types from tissue comprising normal ovarian cells such as ovarian epithelial cells (surface epithelium and/or inclusion cyst epithelium). The cell culture media adapted for growth of normal fallopian tube cells may be used to culture select cell types from tissue comprising normal fallopian tube cells such as fallopian tube epithelial cells (ciliated cells and/or non-ciliated cells fallopian tube cells). The cell culture media adapted for growth of tumor cells, such as ovarian tumor cells, may be used to culture select tumor cell types. Thus, an ovarian tumor tissue comprising an adenocarcinoma, such as at least one of the six major histopathological subtypes (papillary serous, mucinous, endometrioid, clear cell, squamous, and transitional types) may be cultured in a cell culture media adapted for growth of ovarian tumor cells, in order to enrich for a specific cell type. As detailed above, the enriched cells may be provided as a substantially purified culture of cells. In certain embodiments, the cells have any one or more of the characteristics of the cells detailed herein (e.g., proliferative capacity, gene expression, growth characteristics, and the like).

B. Identification of Cultured Cell Types

Following the selective culture of cell types, cell types may be identified using one or more molecular markers. Among normal ovarian cells, the expression pattern of three proteins (PAX8, FOXJ1, and Keratin 7 (CK7) varies among cell types. Ovarian surface epithelium is CK7 (+), PAX 8(−), and FoxJ1 (−), while ovarian inclusion cyst epithelium is CK7 (+), PAX 8(+), and FoxJ1 (−). Similarly, fallopian tube ciliated cells (CK7 (−), PAX 8(−), and FoxJ1 (+)) may be distinguished from fallopian tube non-ciliated cells (CK7 (+), PAX 8(+), and FoxJ1 (−)).

After oncogenic transformation of non-ciliated fallopian tube cells or ovarian inclusion cyst cells, these cell types are associated with expression of probes. Transformed non-ciliated fallopian tube cells overexpress the probesets DOK5, CD47, HS6ST3, DPP6, OSBLP3, and transformed ovarian inclusion cyst cells overexpress the probesets STC2, SFRP1, SLC35F3, SHMT2, TMEM164. Accordingly, a tumor may be identified as more fallopian tube-like or ovary-like. In some embodiments, a kit may comprise a probeset associated with fallopian tube cells, and/or a probeset associated with ovarian cells.

One aspect of the invention relates to a substantially purified culture of ovarian cells and/or fallopian tube cells, wherein the ovarian cells overexpress the probesets DOK5, CD47, HS6ST3, DPP6, OSBLP3, and wherein the fallopian tube cells overexpress the probesets STC2, SFRP1, SLC35F3, SHMT2, TMEM164, and wherein the culture comprises at least 10³ cells, and wherein the cells are capable of undergoing at least 14, 25, or 35 population doublings. In some embodiments, the culture comprises at least 10⁵, 10⁶, 10⁷, 10⁸, 10⁹, and/or 10¹⁰ cells.

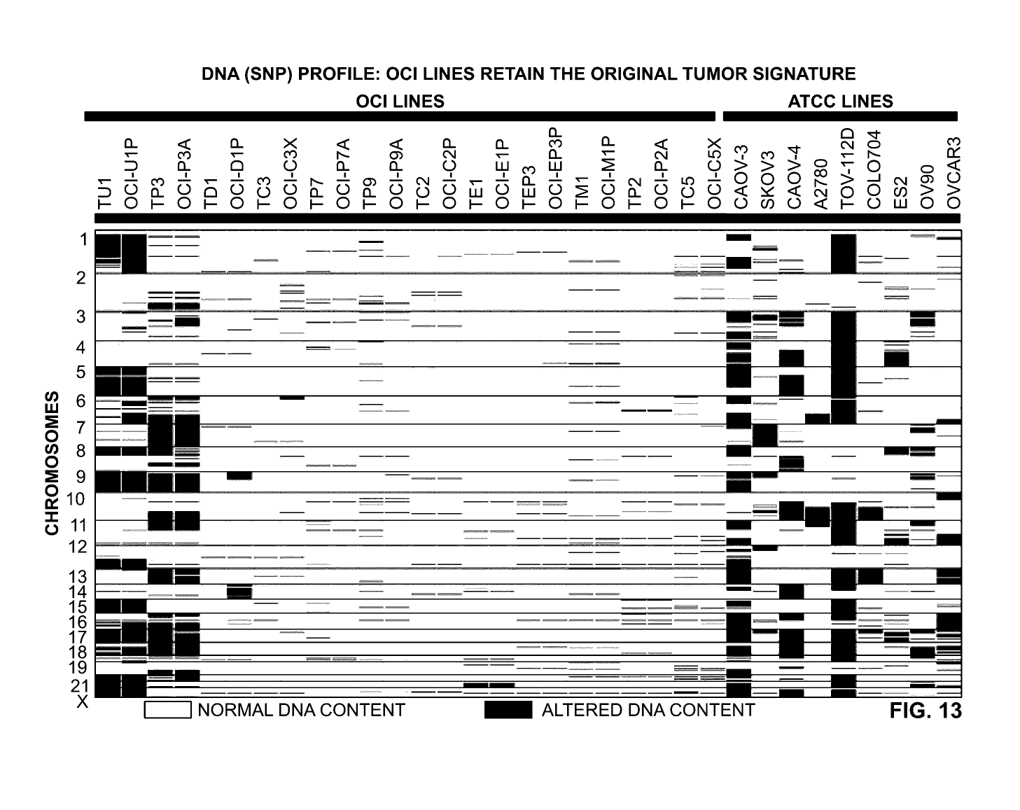

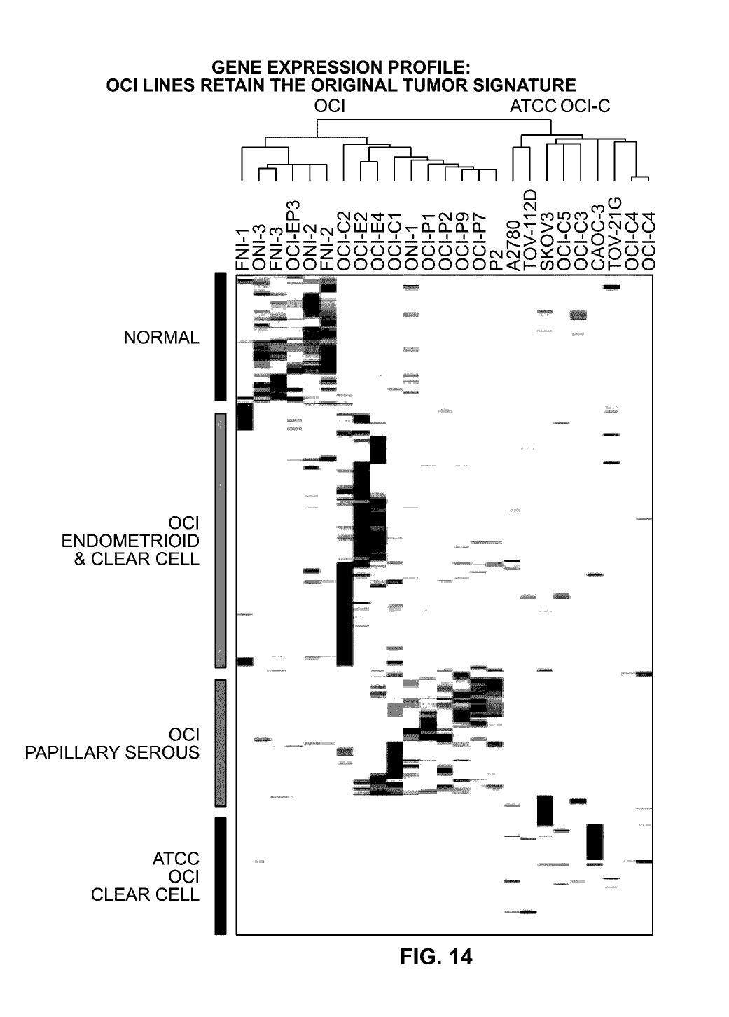

DNA fingerprints may be generated for a line of cultured tumor cells, by generating expression profiles of genomic DNA from the cells. mRNA expression profiles and protein expression profiles may also be created for each tumor cell line. In some embodiments, the disclosure provides ovarian cancer cells with the profiles of FIGS. 13, 14, and/or 15.

As each cell line is tested for drug responsiveness, the expression profiles may be correlated with drug response. In some embodiments, expression profiles may be compared to tumors from patients, and, where knowledge of the patient's prognosis is known, an expression profile from a cultured cell line may indicate the prognosis. In some embodiments, a prediction of a drug's likely effect on a patient's tumor may be established, based on the expression profile of the patient's tumor, and a comparison to a reference cell line for which drug responsiveness is known. For example, if a patient's tumor has an expression profile substantially similar to that of a reference cell, and the reference cell is responsive to a drug treatment, then one can predict that the patient's tumor will also respond to the drug treatment. In some embodiments tumor cells obtained from a biopsy or surgery are cultured and exposed to one or more therapeutic agents in culture. The effect of the agent(s) on proliferation or survival of the cells is assessed in order to obtain a prediction of the one or more agents' likely effect on the subject's tumor. In some embodiments a treatment is selected and/or administered to a subject based at least in part on a prediction or result suggesting that the drug will be beneficial or effective or more effective than one or more alternatives.

C. Exemplary Uses for Cultured Cells

Cultured cells may be used, e.g., in a variety of screening methods, in order to identify candidate agents for cancer therapeutics, candidate agents useful for other therapeutic purposes (e.g., for use in other diseases affecting the ovaries, fallopian tubes, etc.), or other purposes. In some embodiments normal cells are used. In some embodiments tumor cells are used. In some embodiments cells obtained from a subject suffering from a disease of interest (e.g., a disease affecting the reproductive system) are used. A method of identifying candidate therapeutic agents comprises culturing cells, either normal ovarian cells or fallopian tube cells that have been oncogenically transformed, or ovarian tumor cells derived from primary culture, in a suitable media. The cells may then be contacted with an agent and the effects of the agent on cell physiology may be measured. An agent that modulates the physiology of the cells is a candidate therapeutic agent. Cell physiology may comprise the growth properties of the cell, expression of one or more molecular markers, morphology, and/or other cell properties that may be measured and quantified. Candidate therapeutic agents may be biomolecules such as nucleic acids (e.g., short interfering RNA, aptamers, or antisense oligonucleotides) and/or protein, or may be small organic molecules. In certain embodiments, the agents are identified based on, for example, the ability of the agent to inhibit proliferation and/or inhibit survival of the cells. In certain embodiments, the agents are identified based on, for example, the ability of the agent to inhibit the ability of the cells to grow in soft agar.

In an additional step, the cultured cells may be administered or implanted into a test animal, so that a xenograft tumor grows in the test animal. Candidate therapeutic agents may be administered to the test animal, and properties of the xenograft tumor may be measured. Candidate therapeutic agents will modulate the growth, physiology, and/or other properties of the tumor. In particular, candidate therapeutic agents will inhibit growth of the tumor, decrease the rate of growth of the tumor, decrease tumor size, decrease tumor volume and/or decrease metastasis. Thus, in certain embodiments, agents are screened in vivo for their ability to inhibit tumor growth or progression in the context of an animal model.

In some embodiments, cultured cells may be used for regenerative medicine. For example, human cells (typically normal cells) obtained or cultured as described herein may be used in the ex vivo construction of tissues or organs for subsequent implantation into a subject (e.g., a human subject or a non-human subject) or may be implanted to augment or repair diseased, injured, or degenerating tissues or organs. In some embodiments, cells are obtained from a subject, cultured ex vivo, and subsequently implanted into the same subject (autologous). In some embodiments cells from a related or unrelated donor are used.

In some embodiments, cells obtained or cultured as described herein may be subjected to genetic modification. In general, cells can be engineered to express or have increased expression of any one or more genes of interest and/or to have reduced or absent expression of any one or more genes of interest or to have a specific mutation or alteration of interest. For example, in some embodiments, cells, e.g., normal cells, may be genetically modified to express or have increased expression of one or more oncogenes or to have reduced or absent expression of one or more tumor suppressor genes. Genes of interest may encode, e.g., proteins, microRNAs, short hairpin RNAs, etc. One or more vectors (e.g., plasmids, virus vectors, etc.) can be introduced into the cells. Various methods for doing so, such as transfection, viral infection, etc, are known in the art. In some embodiments such genetic modification may be used to generate diseases models.

In some embodiments cultured cells are used in the production of recombinant proteins, e.g., therapeutic proteins.

As is understood in the art, screening typically involves testing one or more agents and comparing the effect of the one or more agents relative to some appropriate control. The use of the control allows the researcher to assess whether the change is significant. In certain embodiments, the cells are used to screen a library of compounds, such as in a high throughput manner.

### EXAMPLES

Having generally described the invention, Applicants refer to the following illustrative examples to help to understand the generally described invention. These specific examples are included merely to illustrate certain aspects and embodiments of the present invention, and they are not intended to limit the invention in any respect. Certain general principles described in the examples, however, may be generally applicable to other aspects or embodiments of the invention. The invention contemplates that any one or more of the aspects, embodiments and other features described above and below can be combined

### Example 1

### Establishment of Normal Ovarian and Fallopian Tube Cultures

a. WIT-Fo Medium:

The normal ovarian epithelial and fallopian tube epithelial cells were cultured in WIT-fo nutrient medium at least 15 population doublings (FIG. 2 A-B, blue line), while replica plates of the same cells under standard media conditions stopped growing after a few passages (FIG. 2 A-B, red line).

b. Standard Ovarian Epithelial Culture Medium:

For ovarian epithelial cells, a control medium composed of a 1:1 mixture of MCDB 105/Medium 199 supplemented with a range of 5-10% fetal bovine serum, 2 mm 1-glutamine and 10 ng/ml epidermal growth factor was used. Dulbecco's modified Eagle's medium (DMEM)/Ham's F-12 (1:1 mixture) with 10-15% fetal bovine serum as a control medium produced similar results. These two media have been used by many other investigators over the past two decades for short term culture of ovarian cells. Ovarian cells were not propagated beyond a few population doublings in these control media. (FIG. 2 A, red line).

c. Standard Fallopian Tube Culture Medium:

For fallopian tube epithelial cells a control medium described by Cromer et al. was used, which is composed of 1:1 mixture of Dulbecco's Modified Eagle's Medium (DME) and Ham's F12, supplemented with 0.1% BSA, 5% serum (1:1 mix of 2.5% fetal bovine serum plus 2.5% Nu Serum) and 17β Estradiol. A slightly modified version of this medium by Levanon et al. was also tested, composed of 1:1 mixture of Dulbecco's Modified Eagle's Medium (DME) and Ham's F12, supplemented with 2% serum. (FIG. 2B, red line).

The ovarian cells grown in WIT-fo medium have reached 14 population doublings in 7 passages (42 days), which is a 8,192-fold increase in net cell number (FIG. 2 B). In standard control medium (DME:F12) the same cells could be passaged 7 times (42 days) as well, however, they only had 2.4 population doublings in 7 passages (42 days) which is equal to 5.3 fold net increase in cell numbers. Thus, there was a 1,526 fold more net increase in the number of cells in WIT-fo than in the standard DME: F12 medium (8,192÷5.3=1,526) (FIG. 2 B).

Several variations of the available cell culture media for culturing ovarian cells were tested. In the media listed below, the ovarian and fallopian tube cells stopped dividing within 3-5 weeks and a net increase in cell numbers was not observed.
MCDB 105/Medium 199 (1:1 mixture) with 10% fetal bovine serum, and 10 ng/ml epidermal growth factor (Karlan et al. American J. of Obs. Gyn. 173 (1), 97-104, 1995).
MCDB 105/Medium 199 (1:1 mixture) with 5% fetal bovine serum (Auersperg et al., Proc. Natl. Acad. Sci. USA Vol. 96, 6249-6254, 1999).
Dulbecco's modified Eagle's medium (DMEM)/Ham's F-12 (1:1 mixture) with 15% fetal bovine serum (Nitta et al, Gynecologic Oncology Volume 81, Issue 1, pp. 10-17, 2001).
MCDB 105/Medium 199 (1:1 mixture) with 10% fetal bovine serum, 2 mm 1-glutamine and 10 ng/ml epidermal growth factor. (Liu at al., Cancer Res. 1; 64(5): 1655-63, 2004).

### Example 2

### Characterization of Normal Ovarian and Fallopian Tube Cells Cultured in WIT-Fo

In order to identify the lineage and origin of the cultured cells, immunohistochemical characterization of normal human ovary and fallopian tube tissues was performed and a marker panel that distinguishes different subsets of epithelial cells in normal fallopian tube and ovarian tissues in vivo was developed.

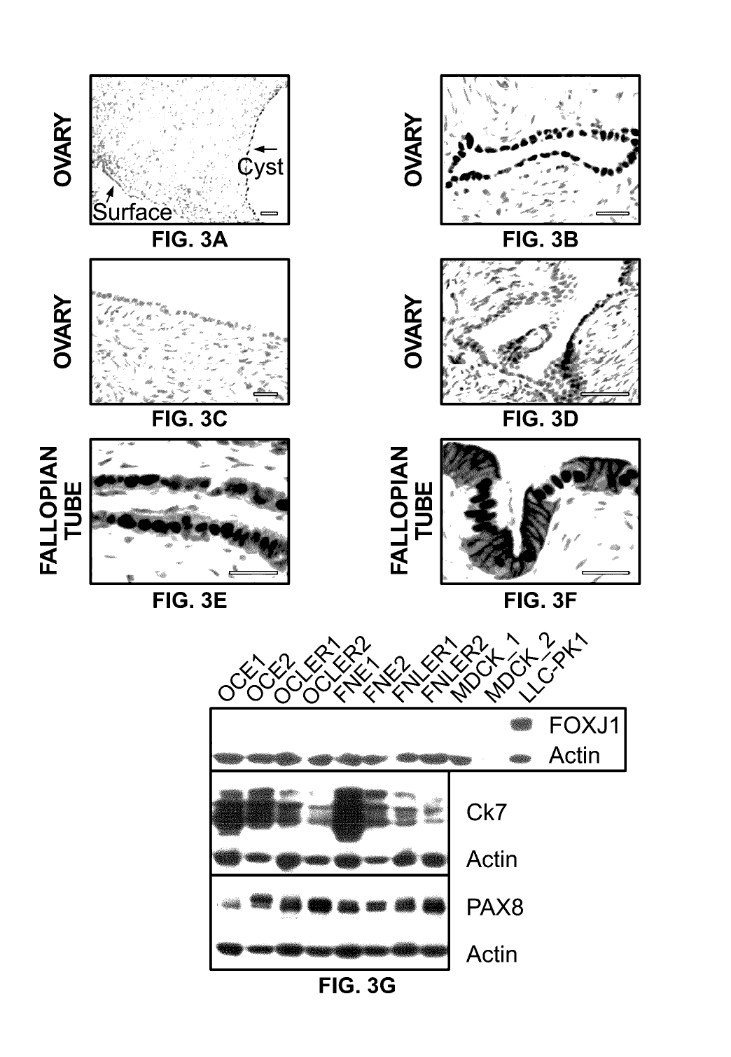

The immunohistochemical examination of formalin-fixed paraffin embedded (FFPE) sections of normal human ovarian and fallopian tube tissues with antibodies that recognize different cells defined normal cell subsets. After screening a number of antibodies, a panel of three antibodies—PAX8, FOXJ1 and Keratin 7 (CK7)—was determined to allow distinguishing between surface vs. inclusion cyst epithelium in the ovary, and ciliated vs. non-ciliated cells in the fallopian tube. All the ovary epithelium and a subset of fallopian tube epithelium were determined to be Keratin 7 positive (+). While the ovarian surface epithelium is PAX8 negative (−), the ovarian inclusion cyst epithelium were PAX8 positive (+). Furthermore, the non-ciliated subset of fallopian tube epithelium were PAX8 positive (+) and FOXJ1 negative (−), distinguishing them from the ciliated cells that were FOXJ1 positive (+) but PAX8 negative (−) (FIG. 3 A-E).

| Tubo-ovarian cell types | Keratin 7 | Pax 8 | FoxJ1 |
|---|---|---|---|
| Ovarian Surface Epithelium | + | − | − |
| Ovarian Inclusion Cyst Epithelium | + | + | − |
| Fallopian Tube Ciliated Cells | − | − | + |
| Fallopian Tube Non-ciliated Cells | + | + | − |

Cells cultured from ovary and fallopian tube were examined. The cells cultured from the ovary expressed CK7 (+), PAX8(+), proteins, but were negative for FOXJ1 (−). This profile is consistent with ovarian inclusion cyst lining. Hereafter, the cultured ovarian cells are referred to as Ovarian Inclusion Cyst (OC) cells (FIG. 3 G).

The cells cultured from normal fallopian tube fimbria expressed CK7 (+), PAX8(+), proteins, but were negative for FOXJ1 (−). This profile is consistent with non-ciliated cells of the fallopian tube epithelium. These cultured cells are referred to as FN cells hereafter (Fallopian tube Non-ciliated cells) (FIG. 3 G).

### Example 3

### Establishment of hTERT Immortalized Normal Ovarian and Fallopian Tube Cultures

a. Immortalization of normal ovarian and fallopian tube cultures. Normal human epithelial cells do not grow well in cell culture and have a finite life span in conventional culture media. In contrast, it is more straightforward to culture all rodent cells and non-epithelial human cell types.

In order to establish continuous normal human epithelial cell cultures, oncogenic transformation is used to create cell lines that can be cultured continuously. In this approach, the oncogenic transformation of normal epithelial cells is achieved by exposing normal human cells to chemical mutagens, radiation or other carcinogenic agents. These agents cause widespread mutations, DNA breaks and chromosomal rearrangements, and cells may no longer be considered as fully normal cells. An alternative method for transforming normal cells uses viral oncogenes to transform normal cells. Among these Human Papilloma Virus E6/7 (HPV E6/7) gene, Simian Virus 40 Large T/small t (SV40T/t) antigen, and adenoviral proteins (E1A), but these methods can be problematic.

A less intrusive method to extend the life-span of normal human cells that does not cause genetic instability or DNA mutations is the over-expression telomerase catalytic subunit (hTERT). hTERT expression actually stabilizes the genome and the cells remain near diploid similar to normal cells.

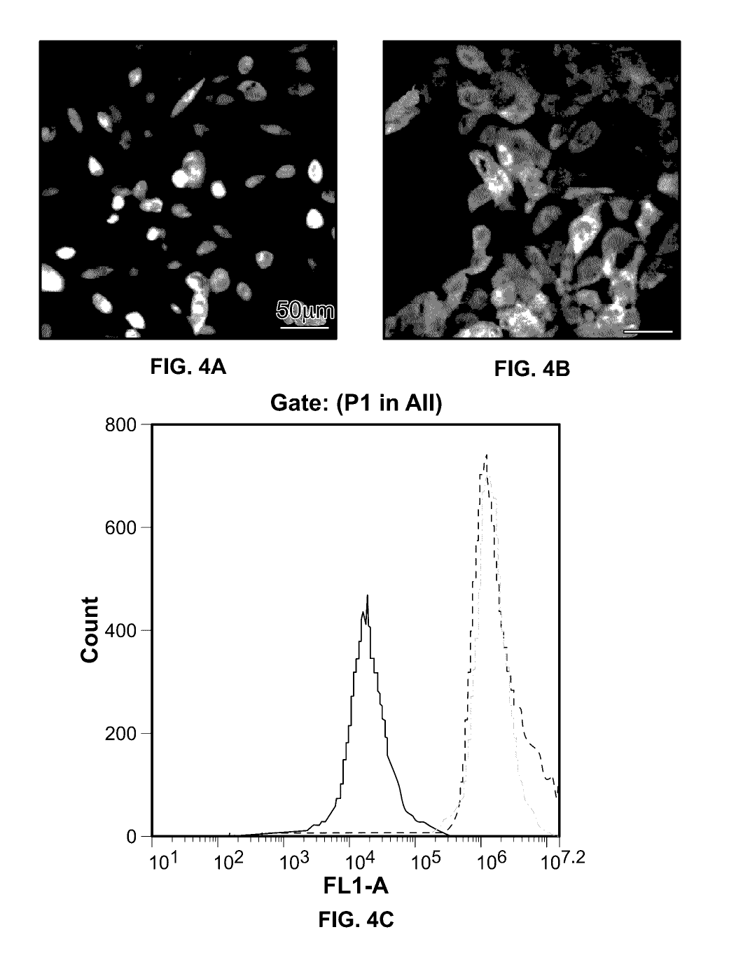

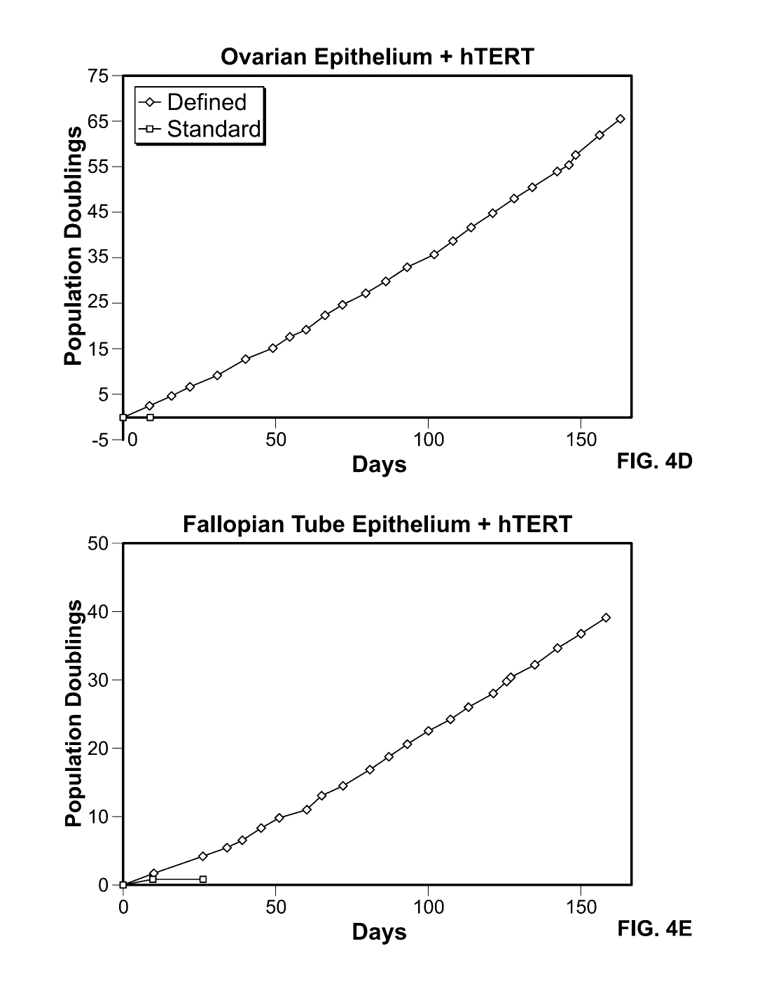

In the WIT-fo medium (see Table 3, column labeled “WIT-fo”), both normal ovarian inclusion cyst (OC) and fallopian tube non-ciliated (FN) epithelial cells were immortalized with hTERT alone. These immortalized OC and FN cells referred to as OCE and FCE cells hereafter respectively (FIG. 3 A-E). These cells were continuously cultured beyond 40 population doublings, a nearly 10¹²-fold net increase in cell numbers, demonstrating their immortalization. In contrast, replica plates of the same cells expressing hTERT under standard media conditions stopped growing after a few passages even in the presence of hTERT over-expression (FIG. 4 A-E).

### Example 4

### Tumorigenic Transformation of Ovarian Inclusion Cyst (OC) and Non-Ciliated Fallopian Tube (FN) Normal Epithelium

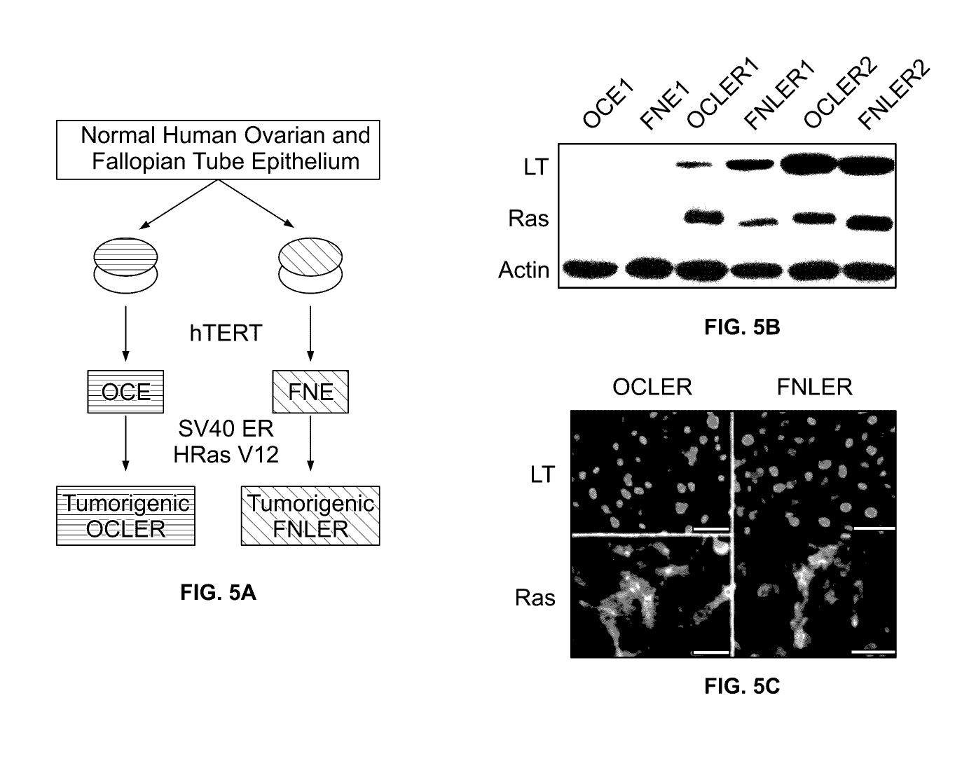

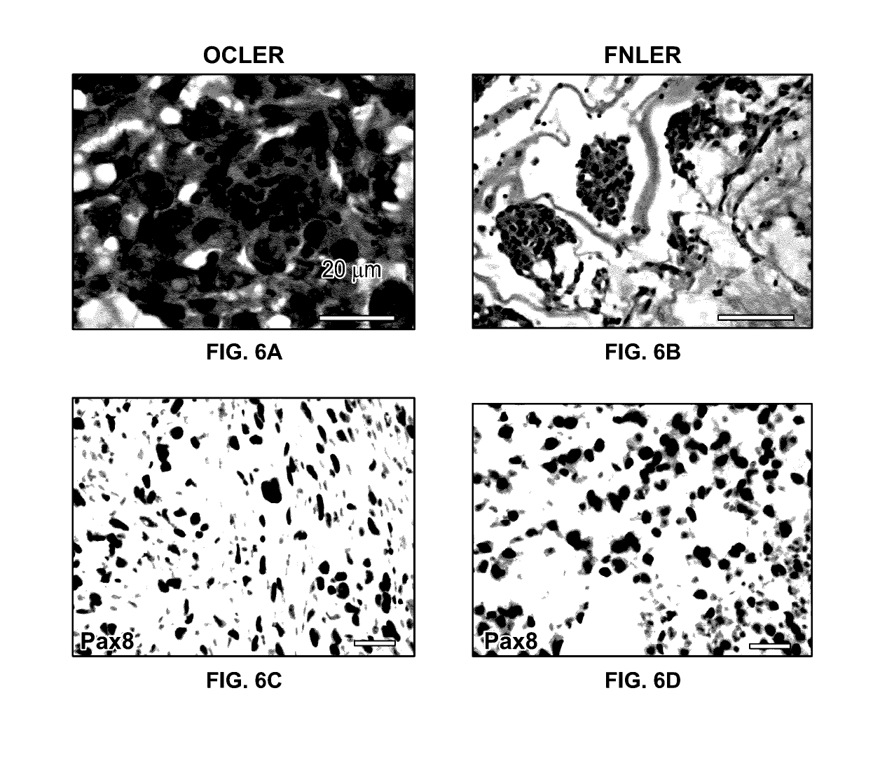

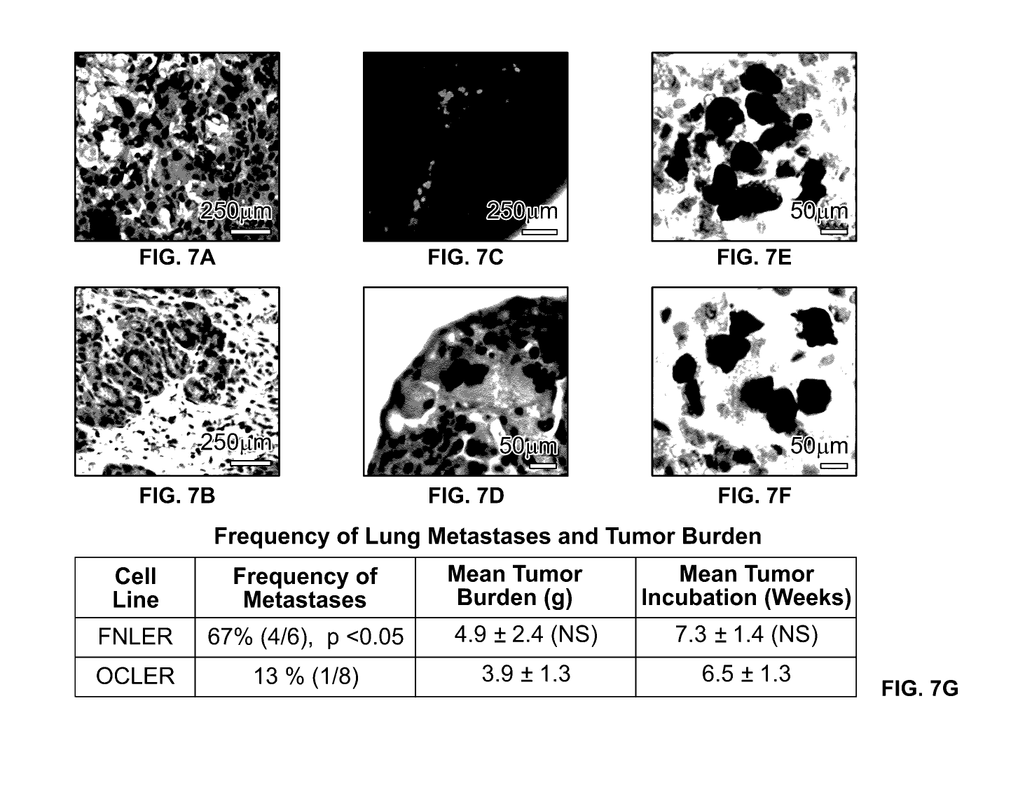

The hTERT immortalized OCE and FNE cells were transformed with SV40T/t and H-Ras as described before (FIG. 5 A). While expression SV40T/t transforms the normal human cells, these cells do not become tumorigenic in mice. Additional expression of an oncogene such as H-Ras makes these transformed cells tumorigenic in mice. The transformed tumorigenic OC cells (OCLER) and FN cells (FNLER) expressed PAX8 and keratin 7 proteins and were negative for FOXJ1 confirming that they retained their original ovarian inclusion cyst and fallopian non-ciliated phenotypes respectively. An equal number of OVLER and FNLER cells were injected into the peritoneal space of immunocompromised nude mice. Necropsy analysis of mice after 5-9 weeks revealed similar size intraperitoneal primary tumors in both groups. Both OCLER and OVLER tumors had a poorly differentiated morphology, however, focally small micopapillary-like structures could be identified (FIG. 6A-B). The tumor cells were also PAX8+(FIG. 6, C-D) and were locally invasive to the surrounding intra peritoneal tissues, such as pancreas (FIG. 7A-B).

Examination of the lungs from tumor bearing mice revealed a striking difference between OC and FN derived tumorigenic cells. The lungs of each mouse was first examined under dissection microscope for gfp+tumor cells (FIG. 7C), and FFPE sections of the same lungs were examined with H&E sections microscopically for atypical tumor cells (FIG. 7D), and with p53 and SV40T/t immunohistochemical stains (FIG. 7 E-F).

The transformed non-ciliated fallopian tube cells (FNLER) formed metastases in the lungs of 67% of the mice (4/6), while isogenic normal ovarian cells transformed with the same oncogenes (OCLER) formed metastases in 13% of the mice (1/8) (FIG. 6G). Since these cells were isolated from the same patient and transformed with identical oncogenes, this suggests a difference in the potential for metastasis in tumors arising in OC vs. FN.

### Example 5

### Discovery of Predictive Cell-of-Origin Gene Expression Signatures

a. Cell origin FNE (fallopian tube) vs. OCE (ovary) expression signature. Using the gene signature derived from these experimental studies, primary ovarian tumor tissues from patients were classified as fallopian tube (FT)-like and ovary (OV)-like. The expression levels of 1,017 probes varied significantly (FDR adjusted P<0.05) between normal immortalized (FNE vs OCE) cells. To produce FT-like and OV-like subdivisions within ovarian cancer datasets, an approach was applied that differs from previous widely used strategies of clustering patients based on global gene expression profiles. Rather, the approach involved selection of the five most highly significant probesets with unique gene symbols that were over-expressed in either FNE (DOK5, CD47, HS6ST3, DPP6, OSBPL3) or OCE (STC2, SFRP1, SLC35F3, SHMT2, TMEM164) and calculation of the sum of the normalized expression values of these genes in two ovarian cancer datasets by weighting FNE genes by (+1) and OCE genes by (−1); specifically, the sum of the normalized expression values of OCE genes were subtracted from the sum of expression values of FNE genes to calculate a score for each tumor. Thus a higher score implies more FT-like. A bimodal distribution of Gaussian curves was fitted using mixture modeling to this score to identify two subpopulations in the dataset; one more OV-like and one more FT-like.

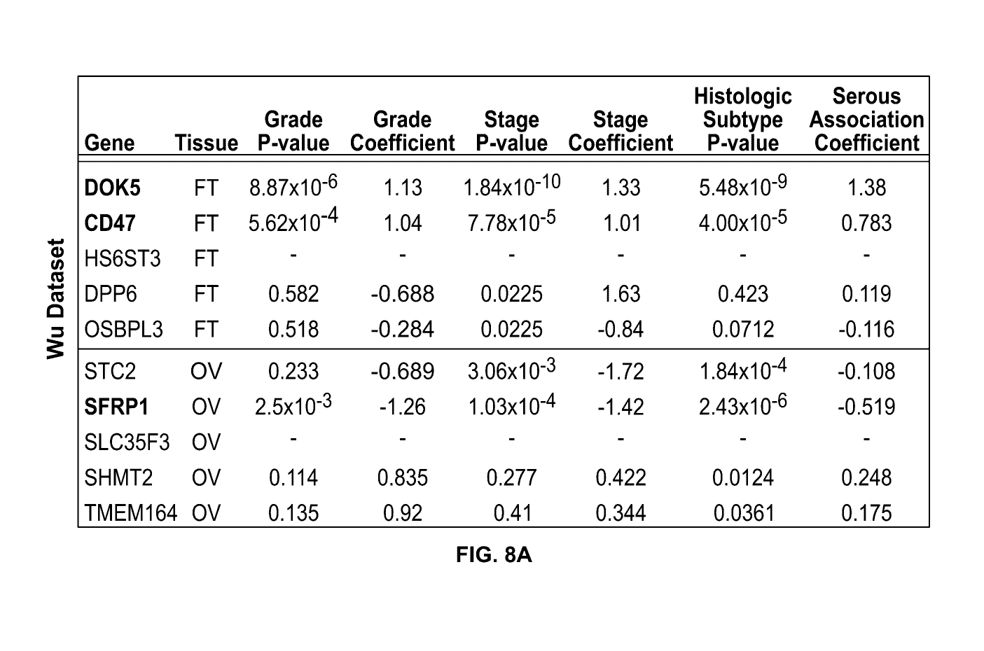

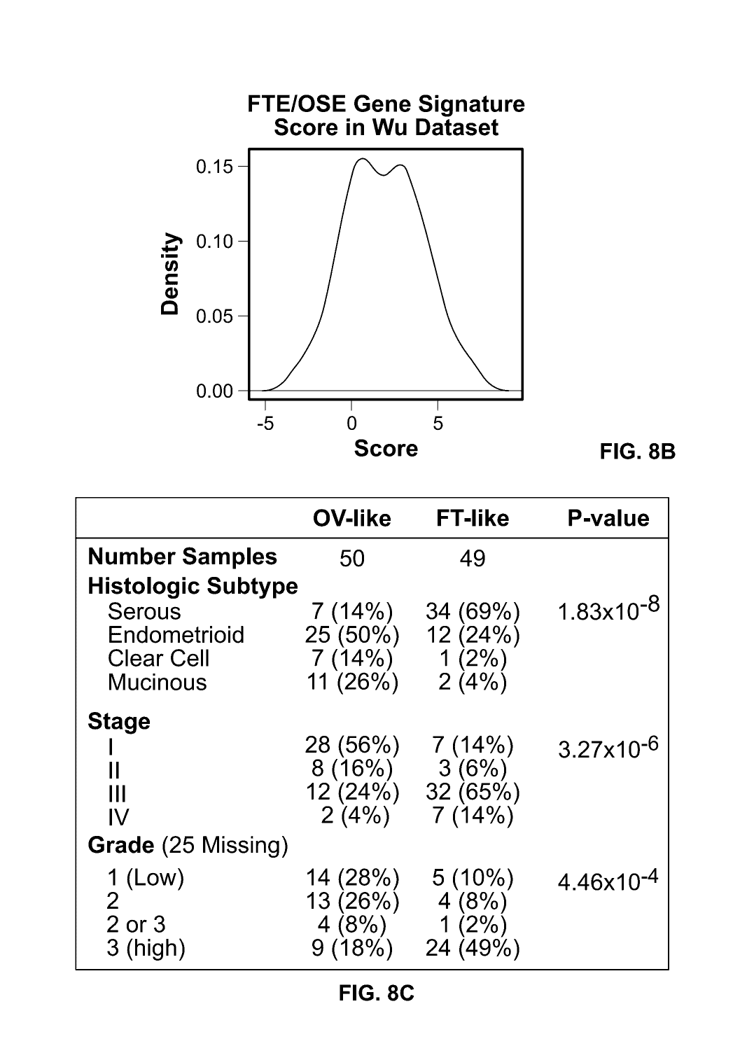

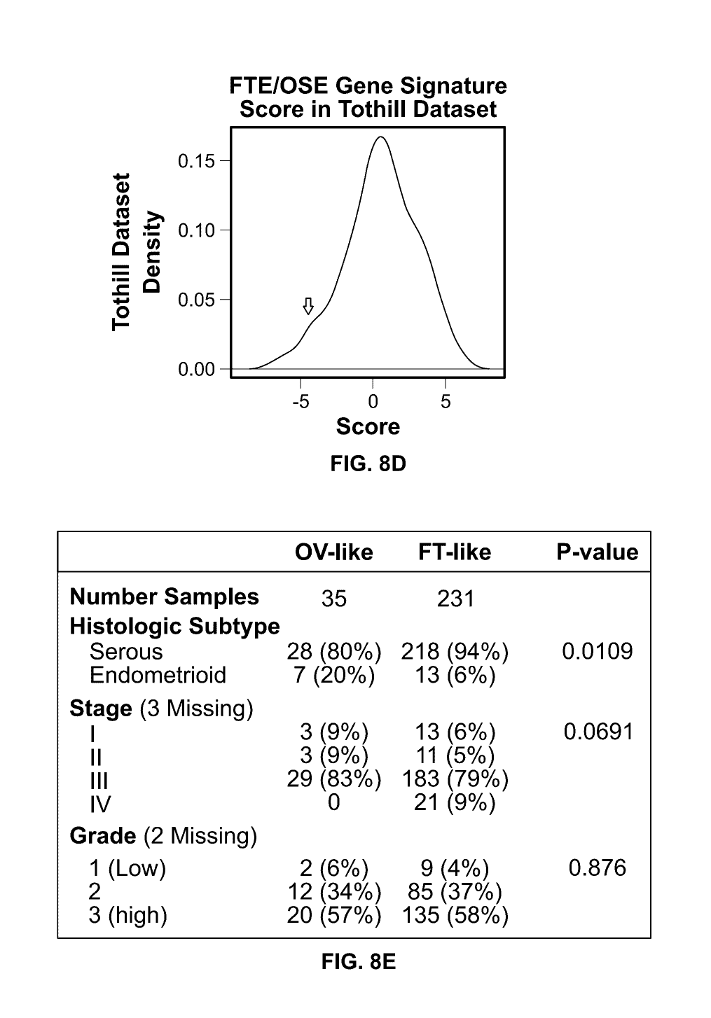

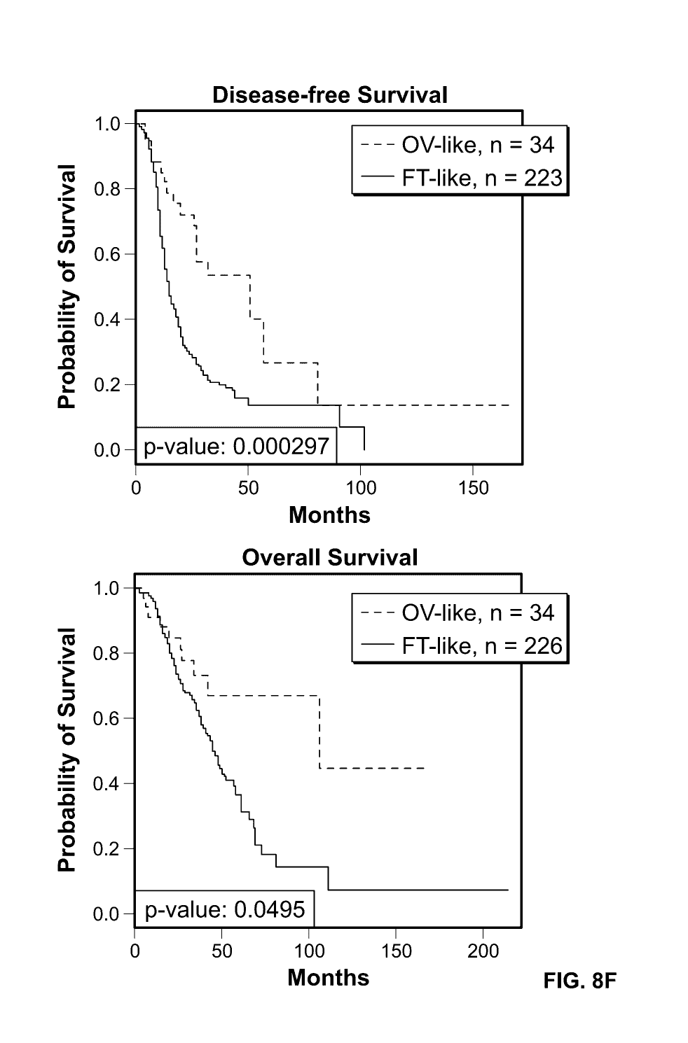

This clustering was performed in the Wu et al. dataset (GEO GSE6008). This study profiled 99 fresh frozen, microdissected epithelial ovarian cancers (including many non-serous histologic subtypes) on a similar array platform. Eight of the 10 genes were available for analysis due to platform differences. In globaltests, these eight genes were strongly related to histologic subtype (P=9.93×10⁻¹¹), grade (P=2.03×10⁻⁷) and stage (P=9.55×10⁻¹²). To identify specific genes that were most strongly associated with these clinical factors, a logistic regression was applied, which revealed that DOK5, CD47, and SFRP1 appeared to be driving this association (FIG. 8 a). The 8-gene signature was used to define FT-like and OV-like subpopulations in the Wu data and visualization of the scores in a density plot illustrates a somewhat bimodal distribution (FIG. 8 b), supporting the segregation into two groups (OV-like and FT-like). Notably, using this scoring system the four normal ovarian surface epithelial samples arrayed in the Wu dataset were all classified as OV-like. The cell-of-origin classification was associated with clinical differences in patient tumors; the FT-like subpopulation included tumors that were of significantly higher stage (P=3.27×⁻⁶), grade (P=4.46×10⁴) and was composed predominantly of serous tumors (P=1.83×10⁻⁸) (FIG. 8 c). In contrast, OV-like tumors included a variety of non-serous histological subtypes, such as endometrioid, clear cell and mucinous tumors, and included more low grade tumors.

The 10-gene signature was further validated in the Tothill dataset (GEO GSE9891) that arrayed fresh frozen tumor pieces (not microdissected) from 246 serous and 20 endometrioid malignant tumors on the same platform; importantly these tissues could be linked with patient survival data. Application of the same Gaussian mixture modeling identified OV-like and FT-like scores that were not clearly bimodal however we observed a slight left skewing which suggests a small subpopulation of OV-like tumors (FIG. 8 d). The FT-like subpopulation was significantly enriched for serous tumors (P=0.0109), contained more high stage tumors (this was non-significant, P=0.0691) and showed no association with grade (P=0.876) (FIG. 8 e). FT-like tumors had significantly worse disease-free survival (P=0.000297) and overall survival (P=0.0495) (FIG. 8 f). Due to the lack of clear bimodality in the Tothill dataset, misclassification of tumors as OV-like or FT-like is possible. However based on the combined results from the Wu and Tothill datasets we concluded that a cell-of-origin signature is present in tubo-ovarian tumors and this classification appears to elucidate previously unrecognized clinically distinct subgroups of tubo-ovarian carcinomas.

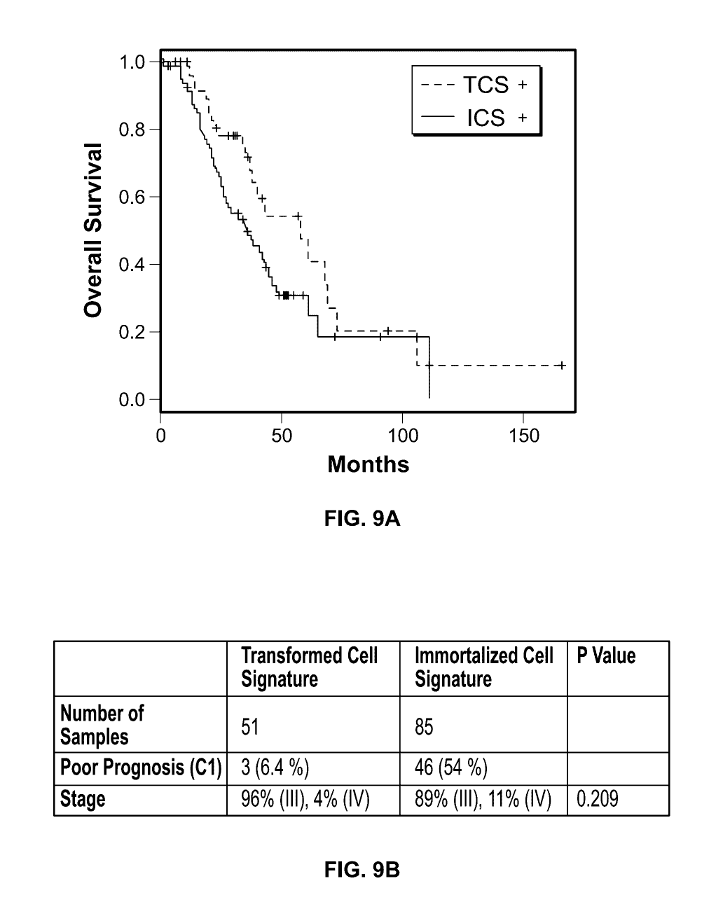

b. Transformed vs. immortalized gene signature. The different cell lines enabled evaluation of gene expression differences between the normal immortalized cell signature (ICS) as compared with their matched transformed cell signature (TCS) irrespective of cell origin (FIG. 9). In order to do this, mRNA expression profiles of immortalized FNE/OCE expressing only hTERT and transformed FNLER/OCLER expressing hTERT+LT/st and H-Ras oncogenes was measured.

The results indicated that ICS+ gene expression signature predicts statistically significant worse clinical outcome in ovarian cancer patients compared to TCS+ovarian cancers (FIG. 9).

Normal immortalized cell signature (ICS) correlated with a worse outcome compared to the transformed cell signature (TCS) (FIG. 9A). This result suggests that the ‘transformed cell signature’ may correlate with a better response to chemotherapy, and predict a better outcome. Microscopic examination of normal epithelium adjacent to tumors almost never shows signs of necrosis or apoptosis after treatment with chemotherapy. In contrast, significant necrosis, apoptosis, inflammation and/or fibrosis is associated with chemotherapy response in tumors. Tumor cells may be more sensitive to chemotherapy because of numerous defective pathways involving DNA damage response, cell cycle regulation, nucleotide and energy metabolism.

Furthermore, a striking overlap was observed between empirically defined poor outcome groups identified by Tothill in their dataset and the ICS signature identified by examining their dataset. Of the 51 cases we classified as ICS, only 3 were classified as Tothill as poor prognosis (C1). In contrast, 46 of 85 ISC tumors were classified as poor prognosis (C1) by Tothill (FIG. 9 B).

### Example 6

### Growth Kinetics of Tumor Cells in Standard Culture Medium

Tumors are complex tissues composed of many cell types; these include many stromal cells such as fibroblasts, endothelial cells, lymphocytes, leukocytes, macrophages, normal epithelial cells etc. that are intermingled with tumor cells. Among these, fibroblasts have been historically the easiest to grow in standard culture medium. When tumor tissue is placed in culture plates, typically there is an explosive growth of fibroblasts such that in a few weeks the fibroblasts completely overtake the culture, and soon all other cells types including tumor cells are eliminated from the culture. Similar results were obtained, in almost all cases using RPMI, DMEM, F12 MCDB105/M199 medium supplemented with serum; the fibroblasts were the major cell type that were proliferating. Hence, in these media the stromal cells completely outgrew the tumor cells, and it was not possible to establish a pure cancer cell line.

In rare samples where the overgrowth of non-tumor cells does not occur, it is possible to achieve short term growth of tumor cells in standard media. However, even in these rare cases of initial success the tumor cells are growth arrested typically after several weeks in standard culture medium (such as DMEM, F12, RPMI and MCDB105/M199), which are most frequently used to culture ovarian cell lines. This is typically followed by wide spread cell death. In most cases no continuous cell line is established and the cultures are abandoned at this point. In very rare cases, a subclone can emerge and give rise to a continuous cell line.

In those rare cases where a continuous tumor cell line can be established in standard medium, generally four stages are observed; brief rapid growth for a few weeks (a), followed by growth plateau (b), followed by wide spread cell death (c) and emergence of a rare cell clone.

This sequence of events is best illustrated in FIG. 10. When a human ovarian tumor sample was plated in RPMI medium, the tumor cells growth arrested around 5 population doublings (FIGS. 10 A and 10C, flat portion of the red and green lines before day 20-40). This was followed by wide spread cell death reflected by decreases in cell numbers (FIGS. 10A and 10C, downward portion of the red and green lines, day 40-50). This kind of growth kinetics is typical of the vast majority of human tumor cultures that are carried out in conventional media.

Only one tumor cell line was established in nearly three dozen attempts using conventional cell culture media. In this one case clone of tumor cells started proliferating in RPMI medium after almost all the other cells were killed as described above (FIG. 10A, red line, day 90).

Significant changes occurred, compared to the uncultured tumor DNA, the genome of the tumor cell clone that arose in RPMI medium after 90 days was examined (FIG. 10B). In contrast, the tumor cells that were grown in WIT-oc were very similar to the original tumor (FIG. 10B).

In summary, with one exception, tumor cells eventually died or the cultures were overtaken by fibroblasts in standard cell culture media, hence, no continuous cell line could be established. Similarly, Verschraegen et al. plated 90 different ovarian carcinoma specimens from 67 patients into RPMI medium, and observed that only eleven out of ninety (12%, 11/90) could be cultured, and only for 15 passages.

Passage Number Vs. Population Doublings:

The number of passages is not equal to population doublings. An example of this is provided in FIG. 10A. Tumor cells were passaged in MCDB-105/M199 for nearly twenty weeks. The typical passage frequency was once a week, equaling to 20 total passages. However, the population doubling curve of these cells was flat after 7 passages. Thus, there was no net increase between passages 7 and 20. Hence, these cells could not provide a practical platform to carry out any meaningful experiments. They were passaged from plate to plate week after week in standard medium with little increase in their cell number.

Lag-Time of Establishing Cultures:

A 90 day lag time was observed before emergence of a cell line in RPMI in our hands (FIG. 10A). This lag time is observed typically due two processes; during this lag period most of the cells die, which in return allows rare subclones that have acquired new mutations to emerge and initiate a fast growing continuous cell line. But as illustrated in FIG. 10, this comes at the expense of genetic alterations that are acquired in cell culture. Thus, such a cell line to be clonal population that significantly deviates from the original tumor cell populations.

### Example 7

### Growth Kinetics of Tumor Cells in WIT-Oc Medium

The growth of tumor cells in WIT-oc medium was immediate, with no lag time. The growth rate was constant, no growth plateau or wide spread cell death was observed (FIGS. 10A and C, blue line). These results suggest that in WIT-oc medium most of the plated tumor cells are able to proliferate without significant in vitro clonal selection (FIGS. 10A and C, blue line). This was typically the growth kinetics of all the cell lines that we established.

Hereafter, the ovarian cancer cell lines that are established in WIT-oc medium as are termed OCI cell lines.

25 continuous OCI cell lines were established; 14 of these were from primary solid human ovarian tissues, 7 continuous OCI cell lines from ascites fluid and 5 OCI cell lines from human tumor xenografts that were initially grown in mice (Table 1).

### Example 8

### Culture of Different Subtypes of Ovarian and Fallopian Tube Tumors

Adenocarcinoma of the ovary is a heterogeneous disease that is comprised of at least six major histopathological subtypes with distinct cellular, morphological, and clinical features. The major subtypes of ovarian adenocarcinoma include papillary serous, mucinous, endometrioid, clear cell, squamous and transitional types, that account for more than ninety percent of ovarian adenocarcinomas. There numerous other rare ovarian tumor subtypes that are mixture of epithelial and mesenchymal phenotypes such as carcinosarcomas, and tumors of germ cell origin such as dysgerminoma.

Continuous tumor cells lines from 6 different subtypes of human ovarian cancer have been established, including tumor types that are particularly difficult to grow such as endometrioid and clear cell cancers (Table 1) and 2. Using fully optimized methods and culture conditions 25 OCI lines in 26 attempts were established, all of these OCI lines were cultured long term (>30 population doublings) with more than 90% success rate. All of the 25 tumor lines were able to form soft agar colonies (Table 2).

In standard culture medium it has not been possible to efficiently grow different subtypes of ovarian cancers. The vast majority of continuous tumor lines were established come from highly aggressive papillary serous ovarian cancers, the other subtypes are rarely cultured.

Moreover, four of the tumor cell lines we established cell lines were from primary fallopian tube carcinomas. Until recently most primary mullerian pelvic tumors were designated as ovarian carcinomas. The culture system we developed was used to establish cell lines from both fallopian tube and ovarian primaries.

### Example 9

### Culture of OCI Cell Lines in Standard Medium

Growth of OCI lines was examined in various standard media. Media recommended for ovarian cancer cells by the American Type Tissue Collection (ATTC), and ECACC was used. Among the seven (7) human ovarian adenocarcinomas available from ATCC, only four three had subtype information available; (1) clear cell carcinoma, one (1) papillary serous carcinoma and one (1) endometrioid carcinoma cell line.

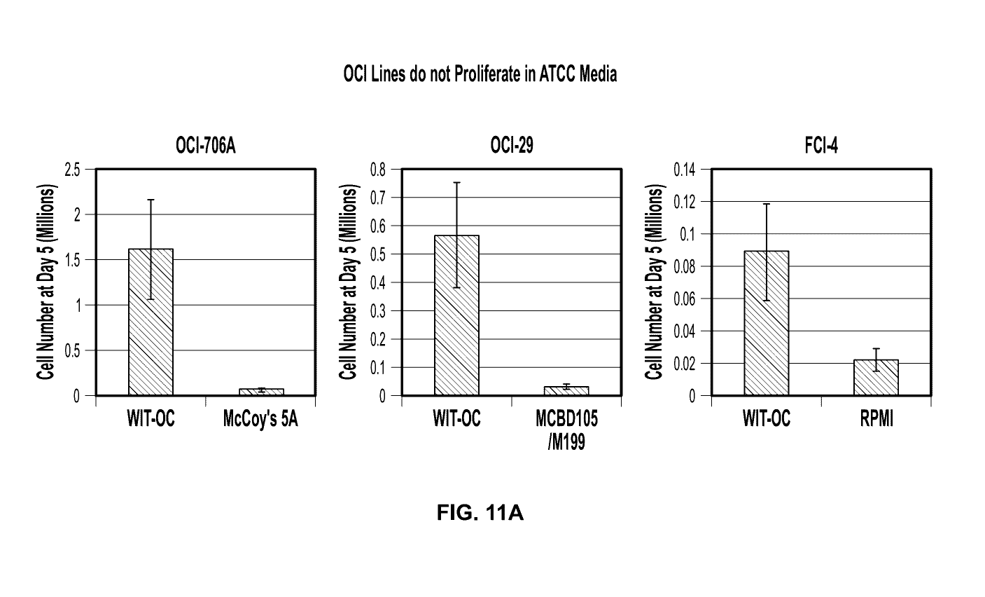

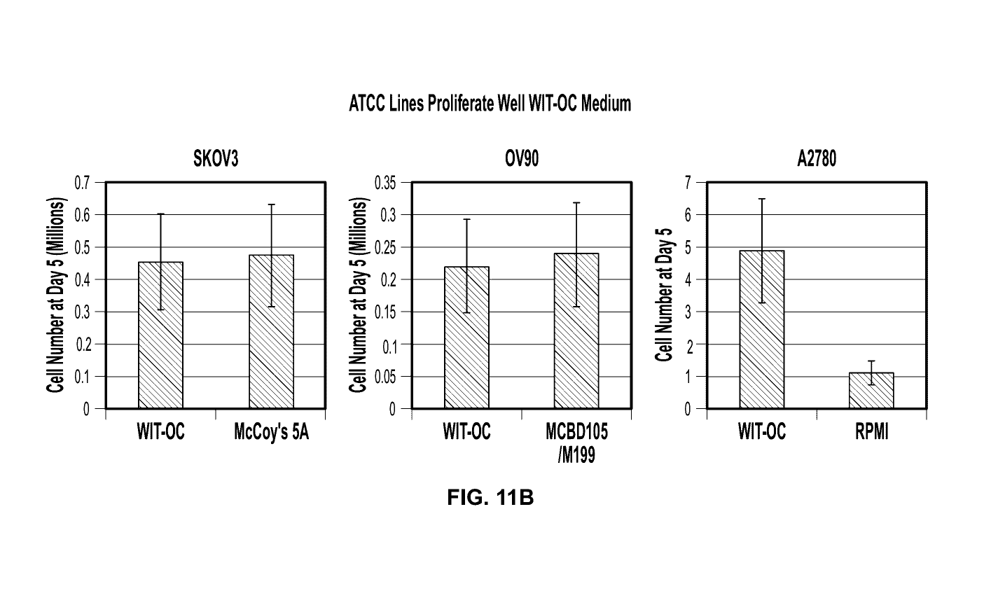

The media recommended for these cells lines by ATCC is MCDB-105/M199, or Dulbecco's Modified Eagle's (DMEM) medium or RPMI medium supplemented with 10-15% serum. The OCI lines could not grow in these media. An example of these experiments with OCI lines is shown in FIG. 11 (top 3 panels). In contrast, all of the ATCC cell lines we tested could be grown in WIT-oc medium (FIG. 11, bottom 3 panels). These results illustrate that standard medium cannot be used to replace WIT-OC.

### Example 10

### Comparison of the Original Tumor Vs. OCI Tumor Line Histopathology

There is a strong relationship between form and function in biology. The specific histopathology (form) of a tumor subtype emerges from a complex interaction between the genetic program of the tumor cells and their microenvironment and systemic factors.

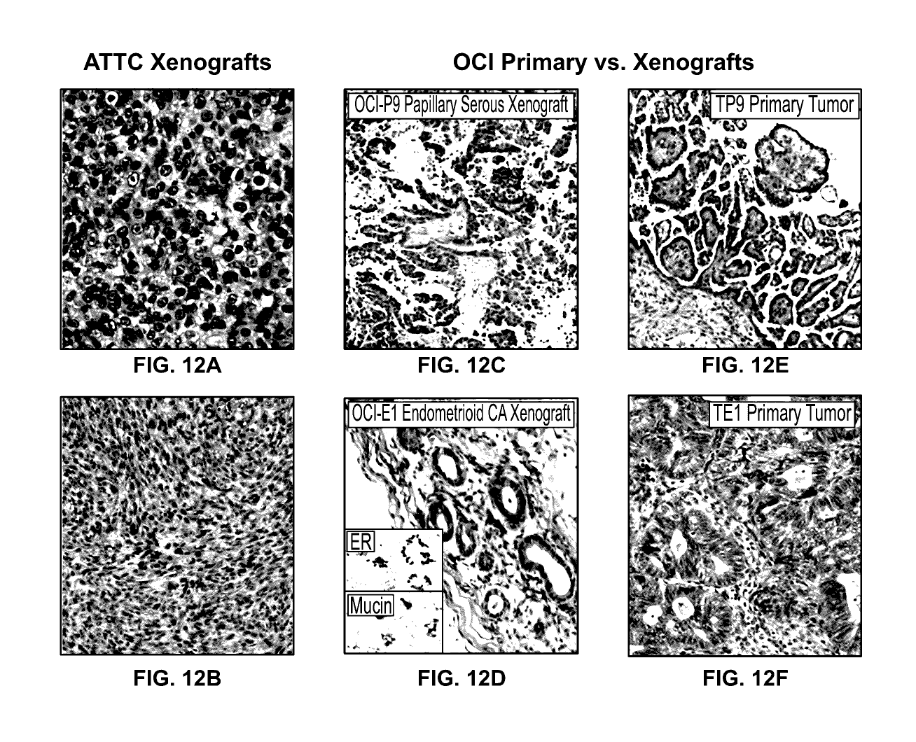

The ATCC/ECACC ovarian tumor lines produce poorly differentiated tumors that do not resemble any human ovarian tumor subtype (FIG. 12A-12B). In contrast, the OCI lines (OCI) that are grown in WIT-oc medium produced tumor xenografts with a histopathology indistinguishable from the original human tumor (FIG. 12C-12F).

In a typical human ovarian papillary serous carcinoma (PSC), the papillary structures are formed by a central stromal core that contains blood vessels. These cores give rise to smaller and smaller papillae that are lined by a malignant epithelium that eventually forms the smallest branches that are formed by clusters of tumor cells with almost no stromal cores (FIG. 12E). This is a very specific architectural feature that is only seen in uterine and ovarian PSCs, and is pathognomic for this tumor. The OCI-P9a line was established from a human PSC, and recapitulated the specific architecture of human PSC when they were injected into immunocompromised mice (FIG. 12C). This is a remarkable result because most xenograft tumors do not recapitulate the histopathologic features of the original tumor.

Another distinct tumor subtype in the ovary is endometrioid adenocarcinoma which typically form back to back glands organized around central lumens. These tumors typically lack papillary structures. These tumors also typically express estrogen receptor and mucins. The OCI-E1p line that was established from an endometrioid adenocarcinoma did form tumors with glandular architecture, expressed estrogen receptor and mucin, recapitulating the original phenotype of the tumor.

In summary, the OCI lines create tumors that are a morphologic phenocopy of human ovarian carcinomas at the histopathologic level, unlike ATCC/ECACC standard ovarian carcinoma lines that do not produce tumor that resemble human ovarian cancers.

### Example 11

### Comparison of the Original Tumor Vs. OCI Tumor Line DNA

The single nucleotide polymorphism (SNP) chips allow a high resolution method to compare the DNA of the cultured ovarian tumor cells and the primary uncultured tumor tissue they originated from. This analysis allows genome-wide detection of chromosomal copy number changes, rearrangements and loss of heterozygosity (LOH).

Genomic DNA of twelve OCI lines and matching original tumor tissue was examined, as well as nine ATCC ovarian carcinoma lines. The genomic DNA from cell lines and their parental tumors were hybridized to Affymetrix 250K SNP arrays and analyzed against a normal reference DNA. These data were plotted in a heatmap in which DNA regions with abnormal changes are highlighted in blue, and the regions with no detectable change are highlighted in yellow. The genome wide pattern of these alternating yellow and blue SNP bands provide a unique DNA fingerprint for each tumor, and allows verification of the cultured tumor cells compared against the original tumor.

There was a remarkable similarity in the CGH profiles of genomic DNA from the uncultured primary tumor tissue and cells grown in WIT-oc. The unsupervised hierarchical clustering of the data shows that each OCI line clusters next to its parental tumor tissue. The OCI lines retained much of the genomic signature of the original tumors, such that they could be identified correctly next to the original tumor DNA reference. Human tumors contain multiple subclones with a spectrum of distinct DNA alterations distinct to each subclone. Thus, the genome wide SNP trace of a tumor is an excellent method to analyze the full genomic heterogeneity of the original tumor. These results indicate that we have been able to retain much of this heterogeneity on cell culture.

The scale of genomic alterations segregated into two distinct groups among OCI lines. Two of the OCI lines (OCI-U1p and P3a) and their matched tumors had large scale alterations that involved up to whole chromosome arms (FIG. 13, red bar, left four lanes). In contrast, the other ten OCI lines and their matched tumors contained genomic alterations that involved narrow regions of the chromosomes (FIG. 13, middle 20 lanes, red bar). These alterations were distributed across the genome, without clear hot spots.

Matching tumor DNA from the patient matching is not publically available for ATCC ovarian tumor lines. Many of these lines were established decades ago, and it is uncertain whether an uncultured piece of tumor tissue can be located for comparison with the original tumor. As a result, ATCC/ECACC lines could not be compared to the original tumor tissue. However, the distribution of the DNA alterations in ATCC/ECACC lines is still informative, because all of the ATCC/ECACC lines without an exception contained large scale Type 1 genomic alterations (FIG. 13, blue bar, right nine lanes), in contrast with majority pattern of OCI lines with a finer small scale alteration pattern.

The SNP profile provides a fingerprint that is different and unique for each tumor, which allows testing and identifying each cell line against the original tumor (FIG. 13).

### Example 12

### mRNA Expression Profile of the OCI Tumor Lines

The mRNA expression profile of OCI lines that were established from different ovarian tumor subtypes had distinct mRNA expression signatures. The mRNA from 12 OCI lines, 5 normal ovary and fallopian lines and 5 ATCC/ECACC lines we hybridized to Affymetrix U133Plus chips. The unsupervised hierarchical clustering of the data revealed that tumor cell lines established from papillary serous, endometrioid and clear cell carcinomas formed separate distinct groups based on their tumor origin (FIG. 14). Furthermore, the mRNA expression signature was distinct from the normal ovarian epithelium (OCE) and fallopian tube epithelium (FNE), and from the ATCC/ECACC ovarian cancer cell lines.

These results confirm that OCI lines retain a significant level of the original tumor gene expression profile that is sufficient to distinguish these tumor types from each other in culture. Furthermore, they indicate that there are significant differences in the expression of hundreds of genes between ATCC lines and OCI lines.

### Example 13

### Protein Expression Profile of OCI Tumor Lines

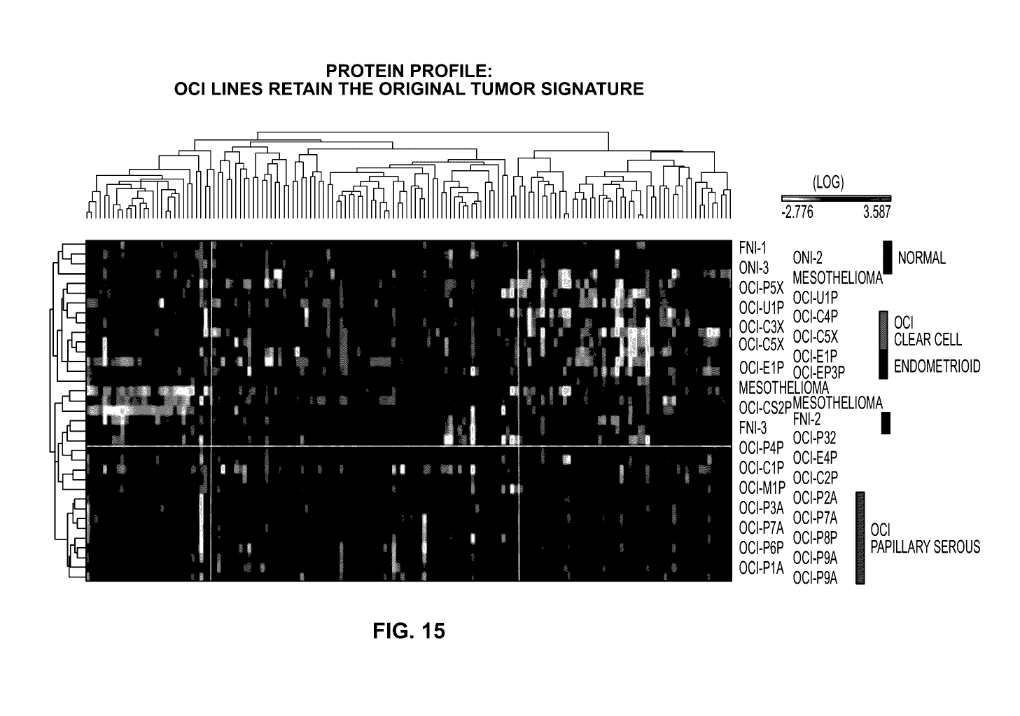

The protein expression profile of OCI lines that are established from different ovarian tumor subtypes had distinct protein expression signatures. The protein extracts of OCI lines were examined with a Reverse Phase Protein Assay (RPPA), which revealed that OCI lines once again clustered according to their tumor origin. The OCI lines established from papillary serous, endometrioid and clear cell carcinomas formed separate clusters (FIG. 15). Furthermore, the protein expression signature was distinct from the normal ovarian epithelium (OCE) and fallopian tube epithelium (FNE), and from the ATCC ovarian cancer cell lines.

These results confirm that OCI lines retain a significant level of the original tumor protein expression profile that is sufficient to distinguish these tumor types from each other and from normal cells correctly in culture. Also, these results indicate that there are significant differences in the expression of dozens of proteins between ATCC/ECACC lines and OCI lines.

### Example 14

### The Drug Response of OCI Lines

As described above, the DNA, RNA and protein profile of the OCI lines are dramatically different than conventional ovarian cancer lines available from the ATCC and ECACC tumor cell line repositories. This raised the possibility that as a result the drug response of the OCI tumor lines may also be different than ATCC/ECACC ovarian tumor lines.

UO126 is an example of a drug that inhibits Mitogen Activated Protein Kinase (MAPK). The MAPK is a member of mitogen-activated kinases, also called “Extracellular Signal-Regulated Kinases” (ERKs). Many growth factors such as EGF and other receptor tyrosine kinases are frequently amplified and over-expressed in cancer activate the MAPK/ERK pathways. In addition, many mutated oncogenes such as Ras activate the MAPK/ERK, which makes this a central pathway in the development of many types of cancer. Hence, many components of the MAPK/ERK pathway are active areas of anti-cancer drug development.

The response of ATCC/ECACC lines (SKOV3, OV90, TOV-1120 and A2780) and the OCI ovarian tumor lines (C5×, P9a1, P7a and FCI-P1p) to MAPK inhibitor UO126 was examined. Both OCI and ATCC/ECACC cell lines were plated in WIT-oc medium (5000 cells/well) into 96 well plates in triplicate. The next day serial dilutions of MAPK inhibitor UO126 was added to the plate. The viable cells were measured with an Alamar Blue assay after 144 hrs with drug incubation, measure as 590/530 florescence.

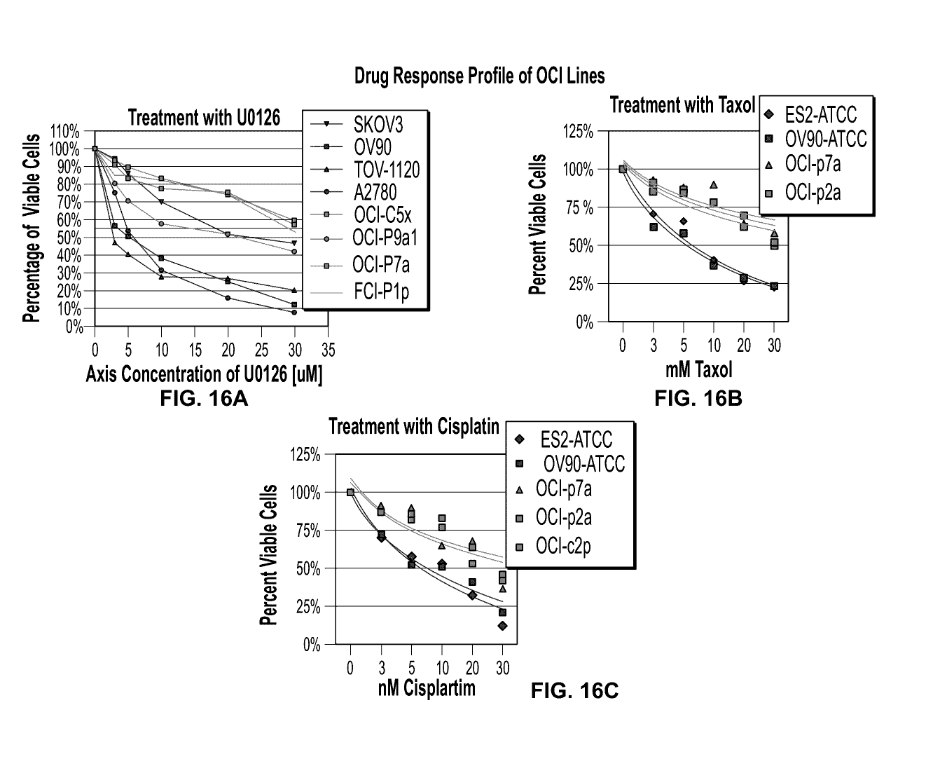

There was a significant difference in the effect of the MAPK inhibitor on OCI cells vs. ATCC/ECACC. As expected, 5 uM of UO126 inhibited proliferation of ATCC/ECACCC cell lines by 50% (LD₅₀). In contrast, approximately 5-fold more UO126 was necessary to achieve 50% inhibition in OCI lines (LD₅₀>30 uM) (FIG. 16A).

Paclitaxel is an example of a drug that inhibits assembly of microtubules. Microtubules are filaments that are part of the cytoskeleton involved in cell division and vesicular transport. Microtubule assembly is required for building the spindle fibers which separates the chromosomes into the daughter cells during mitosis. Therefore, the drugs that inhibit microtubules inhibit the proliferation of tumor cells as well.

The OCI ovarian tumor lines (P7a, P2a, C2p) and ATCC/ECACC ovarian tumor lines (ES2 and OV90) were plated in WIT-oc medium (5000 cells/well) in 96 well plates in triplicate. The next day serial dilutions of paclitaxel were added to the plate. The viable cells were measured as 590/530 florescence with an Alamar Blue assay after 72 hrs with drug incubation.

There was a dramatic difference in the effect of the paclitaxel on OCI cells vs. ATCC/ECACC. The concentration of paclitaxel that produced a 50% decrease in tumor cell numbers (LD₉₀) was more than 5-fold higher in OCI lines compared to ATCCC/ECACC lines (FIG. 16B).

Cisplatin is an example of a chemotherapeutic drug that causes DNA cross linking. Cisplatin binds to DNA and chemically crosslinks the two strands, which causes DNA damage during cell division. This elicits repair mechanisms, which in turn activate apoptosis when repair proves impossible. We plated the OCI ovarian tumor lines (P7a, P2a, C2p) and ATCC/ECACC ovarian tumor lines (ES2 and OV90) in WIT-oc medium (5000 cells/well) in plates in triplicate. The next day serial dilutions of cisplatin were added to the plate. The viable cells were measured as 590/530 florescence with an Alamar Blue assay after 72 hrs with drug incubation.

There was a dramatic difference in the effect of the cisplatin on OCI cell lines vs. ATCC/ECACC cell lines. The concentration of these drugs that produced a 50% decrease in tumor cell numbers (LD₉₀) was more than 6-fold higher in OCI lines compared to ATCCC/ECACC lines (FIG. 7C).

Thus, OCI lines are more resistant to three very different classes of cancer drugs with diverse mechanisms of action, compared to conventional ovarian cancer cell lines (ATCC/ECACC).

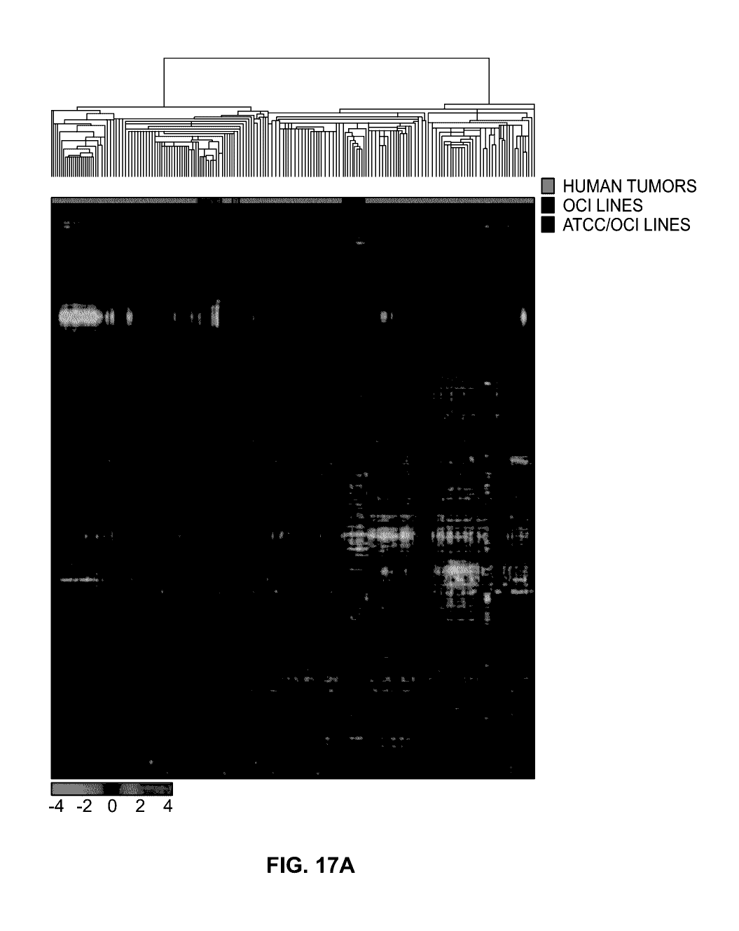

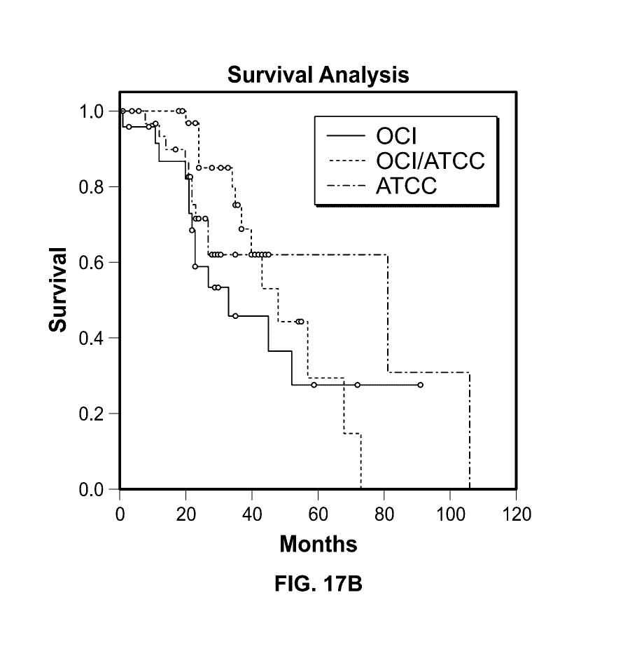

Paclitaxel and cisplatin are commonly used first line drugs in the treatment of ovarian cancer, thus the results described above are highly relevant for development of better treatments. One shortcoming of the existing tumor cell lines is their high level of sensitivity to anti-cancer drugs. This sensitivity leads to many “false positive” results during drug development, i.e.; the drugs that are able to kill cell lines in culture are picked for further pre-clinical and clinical development. However, many of these drugs that are effective on exiting tumor cell lines do not prove effective in the clinic when tested in patients. The lack of correlation between the drug sensitivity of tumor cell lines vs. patients is a major road block for the rapid development of effective cancer treatments. Survival of patients with ovarian tumors with OCI-like gene expression signature: Ovarian tumor patients with a tumor that has an OCI-like gene expression profile have a statistically significantly worse survival than tumors with a ATCC-like expression profile (FIG. 17). This is consistent with the in vitro drug response results described above in which OCI lines were more resistant to drugs that are most commonly used to treat ovarian carcinoma patients, such as paclitaxel and cisplatin. These results suggest that OCI lines do provide more relevant information about the treatment response of patients, compared to existing tumor cell lines.

### Example 15

### Tumor Types Other than Tubo-Ovary Origin have been Successfully Grown in WIT-Oc Medium

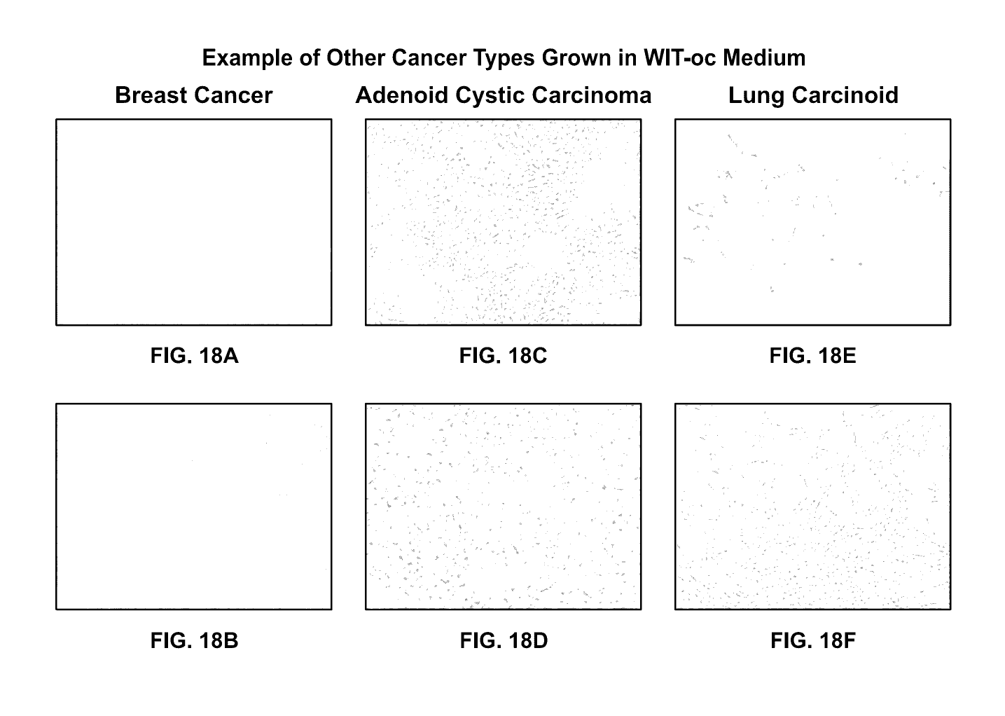

Several tumor samples were removed from human breast cancers, pancreatic cancer, adenoid cystic carcinoma, neuroendocrine tumor (carcinoid) from the lung or from the gastrointestinal tract and tested in WIT-oc medium. The methods and the culture medium described here enables the culture of these tumors (FIG. 18).

### Tables

Table 1. Summary of the number of continuous ovarian tumor cell lines established from different tumor subtypes. This table shows the number of primary tubo-ovarian carcinoma cell lines that were established from primary solid tumors, malignant ascites fluid or from xenograft tumors as well as from different tubo-ovarian subtypes are listed.

TABLE 1

| | | Tissue Source | | | |
|---|---|---|---|---|---|
| Ovarian Cancer (OCI) Lines | | Primary | Ascites | Xenograft | Total |
| Tumor | Papillary Serous | 7 | 6 | 2 | 15 |
| Type | Clear Cell | 2 | 1 | 2 | 4 |
| | Endometrioid | 2 | | 1 | 3 |
| | Mucinous | 1 | | | 1 |
| | Germ Cell | 1 | | | 1 |
| | Carcinosarcoma | 1 | | | 1 |
| | TOTAL | 14 | 7 | 5 | 25 |

Table 2. List of continuous ovarian tumor cell lines established from different tumor subtypes. This table shows the list of 25 tubo-ovarian carcinoma cell lines that were established from primary solid tumors, malignant ascites fluid or from xenograft tumors as well as from different tubo-ovarian subtypes are listed.

TABLE 2

| | | | | | Soft Agar | | |
|---|---|---|---|---|---|---|---|
| | Cell Line | Histopathological | Sample | Primary | Colony | Fresh | Frozen |
| | Name | Subtype | Origin | Tumor Site | Formation | Sample | Sample |
| 1 | OCI-C1p | clear cell | Primary | Ovary | + | | + |
| 2 | OCI-C2p | clear cell | Primary | Ovary | + | | + |
| 3 | OCI-C3x | clear cell | Xenograft | Ovary | + | + | |
| 4 | OCI-C4p | clear cell | Primary | Ovary | + | + | |
| 5 | OCI-C5x | clear cell | Xenograft | Ovary | + | | + |
| 6 | OCI-P1a | papillary serous | Ascites | Ovary | + | + | |
| 7 | OCI-P2a | papillary serous | Ascites | Ovary | + | + | |
| 8 | OCI-P3a | papillary serous | Ascites | Ovary | + | + | |
| 9 | OCI-P4p | papillary serous | Primary | Ovary | + | + | |
| 10 | OCI-P5x | papillary serous | Xenograft | Ovary | + | + | |
| 11 | OCI-P6p | papillary serous | Primary | Ovary | + | + | |
| 12 | OCI-P7a | papillary serous | Ascites | Ovary | + | + | |
| 13 | OCI-P8p | papillary serous | Primary | Omentum | + | + | |
| 14 | OCI-P9a1 | papillary serous | Ascites | Ovary | + | + | |
| 15 | OCI-P9a2 | papillary serous | Ascites | Ovary | + | + | |
| 16 | FCI-P1p | papillary serous | Primary | Fallopian | + | + | |
| | | | | Tube | | | |
| 17 | FCI-P2p | papillary serous | Primary | Fallopian | + | + | |
| | | | | Tube | | | |
| 18 | OCI-E1p | endometrioid | Primary | Ovary | + | + | |
| 19 | OCI-E2p | endometrioid | Primary | Ovary | + | + | |
| 20 | OCI-E4 | endometrioid | unknown | Ovary | + | | + |
| 21 | OCI- | Mixed | Primary | Ovary | + | + | |
| | EP3p | (endometrioid/pap | | | | | |
| | | serous) | | | | | |
| 22 | OCI-M1p | mucinous | Primary | Ovary | + | + | |
| 23 | OCI-U1a | mullerian (not | Ascites | Ovary | + | + | |
| | | specified) | | | | | |
| 24 | OCI-CSp | carcinosarcoma | Primary | Ovary | + | + | |
| 25 | OCI-D1p | dysgerminoma | Primary | Ovary | + | | + |

TABLE 3
Exemplary formulations

| Medium Name | WIT-oc | | WIT-fo |
|---|---|---|---|
| Tissue | Papillary | Endometrioid | Normal |
| | Serous | Tumor | Fallopian |
| | Tumor | | Tube Cells |
| Tissue | Clear Cell | Mucinous | Normal |
| | Tumor | Tumor | Ovarian |
| | | | Cells |
| Tissue | Carcino- | | |
| | sarcoma | | |
| Tissue | Dysgerminoma | | |
| Concentration | mg/L | mg/L | mg/L |
| Serum | 1.8% | 1.8% | 0.5% |
| Estrogen | none | 100 nM | none |
| (B-17 Estradiol) | | | |
| Hydrocortisone | 0.15 | 0.15 | 0.50 |
| EGF | 0.01 | 0.01 | 0.01 |
| Insulin | 15.00 | 15.00 | 20.00 |
| Choleratoxin | 0.20 | 0.20 | 0.20 |
| 2-deoxy-D-ribose | 0.25 | 0.25 | 0.25 |
| Adenine sulfate | 5.00 | 5.00 | 5.00 |
| Adenosine | 0.10 | 0.10 | 0.10 |
| 5′-phosphate | | | |
| Adenosine | 0.50 | 0.50 | 0.50 |
| 5′-triphosphate | | | |
| Alpha-tocopherol | 0.01 | 0.01 | 0.01 |
| Phosphate | | | |
| Ascorbic acid | 0.03 | 0.03 | 0.03 |
| Biotin | 0.01 | 0.01 | 0.01 |
| BSA | 1250.00 | 1250.00 | 1250.00 |
| Calciferol | 0.05 | 0.05 | 0.05 |
| (Vitamin D2) | | | |
| Calcium chloride | 116.61 | 116.61 | 116.61 |
| (CaCl2) | | | |
| Cholesterol | 0.10 | 0.10 | 0.10 |
| Choline chloride | 7.25 | 7.25 | 7.25 |
| Cupric sulfate | 0.00 | 0.00 | 0.00 |
| (CuSO4—5H2O) | | | |
| D-Calcium | 0.26 | 0.26 | 0.26 |
| pantothenate | | | |
| D-Glucose | 1401.00 | 1401.00 | 1401.00 |
| Ferric nitrate | 0.35 | 0.35 | 0.35 |
| (Fe(NO3)—9H2O) | | | |
| Ferric sulfate | 0.42 | 0.42 | 0.42 |
| (FeSO4—7H2O) | | | |
| Folic acid | 0.66 | 0.66 | 0.66 |
| Glutamine | 292.00 | 292.00 | 292.00 |
| Glutathione | 0.03 | 0.03 | 0.03 |
| (reduced) | | | |
| Glycine | 28.75 | 28.75 | 28.75 |
| Guanine | 0.15 | 0.15 | 0.15 |
| hydrochloride | | | |
| Hepes | 2380.00 | 2380.00 | 2380.00 |
| Hypoxanthine-Na | 2.59 | 2.59 | 2.59 |
| i-Inositol | 9.03 | 9.03 | 9.03 |
| L-Alanine | 16.95 | 16.95 | 16.95 |
| L-Arginine | 140.50 | 140.50 | 140.50 |
| hydrochloride | | | |
| L-Asparagine-H2O | 7.51 | 7.51 | 7.51 |
| L-Aspartic acid | 21.65 | 21.65 | 21.65 |
| L-Cysteine | 17.61 | 17.61 | 17.61 |
| HCl—H2O | | | |
| L-Cystine-2HCl | 13.00 | 13.00 | 13.00 |
| L-Glutamic Acid | 44.85 | 44.85 | 44.85 |
| L-Glutamine | 123.00 | 123.00 | 123.00 |
| L-Histidine - | 21.44 | 21.44 | 21.44 |
| HCl—H2O | | | |
| L-Hydroxyproline | 5.00 | 5.00 | 5.00 |
| Linoleic Acid | 5.00 | 5.00 | 5.00 |
| Lipoic Acid | 0.11 | 0.11 | 0.11 |
| L-Isoleucine | 22.00 | 22.00 | 22.00 |
| L-Leucine | 36.55 | 36.55 | 36.55 |
| L-Lysine | 53.25 | 53.25 | 53.25 |
| hydrochloride | | | |
| L-Methionine | 9.75 | 9.75 | 9.75 |
| L-Phenylalanine | 15.00 | 15.00 | 15.00 |
| L-Proline | 37.25 | 37.25 | 37.25 |
| L-Serine | 17.75 | 17.75 | 17.75 |
| L-Threonine | 20.95 | 20.95 | 20.95 |
| L-Tryptophan | 6.02 | 6.02 | 6.02 |
| L-Tyrosine 2Na | 23.91 | 23.91 | 23.91 |
| 2H20 | | | |
| L-Valine | 18.35 | 18.35 | 18.35 |
| Magnesium chloride | 28.61 | 28.61 | 28.61 |
| Magnesium sulfate | 48.84 | 48.84 | 48.84 |
| (MgSO4) | | | |
| Menadione | 0.01 | 0.01 | 0.01 |
| (Vitamin K3) | | | |
| Niacin | 0.01 | 0.01 | 0.01 |
| Niacinamide | 0.03 | 0.03 | 0.03 |
| Para-aminobenzoic | 0.03 | 0.03 | 0.03 |
| acid | | | |
| Phenol red | 10.60 | 10.60 | 10.60 |
| Phosphoethanolamine | 5.00 | 5.00 | 5.00 |
| Potassium chloride | 311.80 | 311.80 | 311.80 |
| (KCl) | | | |
| Putrescine-2HCl | 0.08 | 0.08 | 0.08 |
| Pyridoxal | 0.01 | 0.01 | 0.01 |
| hydrochloride | | | |
| Pyridoxine | 0.04 | 0.04 | 0.04 |
| hydrochloride | | | |
| Riboflavin | 0.02 | 0.02 | 0.02 |
| Ribose | 0.25 | 0.25 | 0.25 |
| Selenous acid | 0.01 | 0.01 | 0.01 |
| Sodium acetate | 25.00 | 25.00 | 25.00 |
| Sodium bicarbonate | 1688.00 | 1688.00 | 1688.00 |
| (NaHCO3) | | | |
| Sodium chloride | 7199.50 | 7199.50 | 7199.50 |
| (NaCl) | | | |
| Sodium phosphate, | 71.00 | 71.00 | 71.00 |
| dibasic | | | |
| Sodium phosphate, | 70.00 | 70.00 | 70.00 |
| monobasic | | | |
| Sodium Pyruvate | 55.00 | 55.00 | 55.00 |
| Thiamine | 0.16 | 0.16 | 0.16 |
| hydrochloride | | | |
| Thymidine | 0.35 | 0.35 | 0.35 |
| Thymine | 0.15 | 0.15 | 0.15 |
| Transferrine | 11.25 | 11.25 | 11.25 |
| Triiodothyronine | 0.00000016 | 0.00000016 | 0.00000016 |
| Tween 80 | 10.00 | 10.00 | 10.00 |
| Uracil | 0.15 | 0.15 | 0.15 |
| Vitamin A (acetate) | 0.05 | 0.05 | 0.05 |
| Vitamin B12 | 0.70 | 0.70 | 0.70 |
| Xanthine-Na | 0.17 | 0.17 | 0.17 |
| Zinc sulfate | 0.43 | 0.43 | 0.43 |
| (ZnSO4—7H2O) | | | |

### Materials & Methods

1) Tissue Collection

Brushings from the normal ovarian surface and the fimbriated end of the fallopian tube were collected using a surgical kit, from patients undergoing surgery for benign gynecologic conditions. The two donor patients were 56 and 65 years old and did not have any type of gynecologic cancer.

2) Isolation and Culture of Ovarian Surface and Fallopian Tube Epithelium

Cells were suspended in a cell culture media and transferred to a tissue culture flask with a modified surface treatment (Primaria, BD Biosciences, Bedford, Mass.) and incubated at 37° with 5% CO2. After 10-15 days, during which the medium was changed every 2-3 days, cells were lifted using 0.05% trypsin and subcultures were established by seeding cells at a minimum density of 1×10⁴/cm². Trypsin was inactivated using medium containing 10% serum, followed by centrifugation of cells in polypropylene tubes (500×g, 4 minutes) to remove excess trypsin and serum. The nutrient medium was replaced 24 hrs after re-plating cells and every 48-72 hours thereafter. Normal ovarian surface and fallopian tube epithelium were cultured in nutrient medium that was optimal for these cells (WIT-fo).

3) Retroviral Infections

Amphotropic retroviruses were produced by transfection of the 293T producer cell line with 1 μg of retroviral vector and 1 μg total of the packaging (pUMVC3) and envelope (pCMV-VSV-G) plasmid at an 8:1 ratio using Fugene 6 (Roche Applied Science, Indianapolis, Ind.). The viral supernatants were harvested at 24 and 48 hrs and used to infect primary ovarian surface and fallopian tube epithelial cells with 8 μg/ml polybrene. Retroviruses were introduced to recipient cells in individual steps in the following order: pmig-GFP-hTERT, pBABE-zeo-SV40-ER and pBABE-puro-H-ras V12. Selection of infected cells was performed serially and drug selection (500 μg/ml zeocin (zeo) and 1 μg/ml puromycin (puro)) was used to purify polyclonal infected populations after each infection. Primary ovarian surface epithelial cells were immortalized with hTERT between passages 2 to 6 and transformed between passages 26 to 30. Primary fallopian tube epithelial cells were transduced with hTERT between passages 1 to 4 and transformed at passage 16. Cells immortalized with hTERT and those that were transduced with SV40 and/or H-ras were cultured in defined media on Primaria tissue culture ware. All protocols involving the use of retroviruses were approved by the Committee on Microbiological Safety.

The immortalized ovarian surface and fallopian tube epithelial cell lines (containing only the pmig-GFP-hTERT vector) will hereafter be referred to as OCE and FNE and fully transformed derivatives as OCLER and FNLER following the introduction of vectors encoding hTERT (E), SV40 early region (L) and HRas (R).

4) Western Blotting

Protein expression was determined by immunoblotting by separation of 30 micrograms of total cell protein on 4-12% NuPAGE Bis-Tris gels (Invitrogen, Carlsbad, Calif.) that were subsequently transferred onto PVDF membranes. Membranes were probed with primary antibodies specific for SV40 LT (Pab 101) (Santa Cruz Biotechnology Inc, Santa Cruz, Calif.), Ras (clone RAS10) and Cytokeratin 7 (MAB3554) (Millipore, Billerica, Mass.), PAX8 (10336-1-AP, ProteinTech Group, Inc, Chicago, Ill.), FOXJ1 (HPA005714) and (β-Actin (clone AC-15) (Sigma-Aldrich, St. Louis, Mo.).

5) Immunofluorescence

Protein expression of cells grown for two days on untreated 12 mm glass coverslips (Warner Instruments, Hamden, Conn.) was measured by immunofluorescence following fixation with 2% paraformaldehyde/0.1% Triton X-100 and 2% paraformaldehyde/acetone for SV40 and Ras, respectively, and incubation with primary antibodies specific for SV40 LT (sc-147, Santa Cruz Biotechnology) and Ras (clone Ras10, Millipore). Images were taken at 40× magnification with oil immersion using a Nikon TE2000-U inverted microscope and SPOT-RT software (Diagnostic Instruments, Sterling Heights, Mich.).

6) Live Cell Imaging and Fluorescence Activated Cell Sorting

Cells were grown for two days on untreated fluorodishes (World Precision Instruments, Sarasota, Fla.) and images of live cells were taken at 40× magnification with oil immersion using the Nikon TE2000-U inverted microscope and EZ-C1 software (Nikon) for image acquisition. Fluorescence activated cell sorting (FACS) analysis using a FACS Aria multicolor high speed sorter (BD Biosciences, San Jose, Calif.) was applied to determine the proportion of ovarian and fallopian tube cells that were GFP positive following infection with pmig-GFP-hTERT.

7) Analysis of Tumorigenicity and Metastasis

The protocol for tumorigenesis experiments in immunocompromised mice was approved by the Harvard Standing Committee on Animals. All experiments were performed in compliance with relevant institutional and national guidelines and regulations. Single-cell suspensions were prepared in a Matrigel: WIT-fo mixture (1:1) and 1 million cells per 100 μl volume were injected in one intraperitoneal and two subcutaneous sites per mouse. Tumor cell injections were performed on 6-8 week old female immunodeficient nude (Nu/Nu) mice (Charles River Laboratories International, Inc, Wilmington, Mass.). Tumors were harvested 5 to 9 weeks after implantation of tumorigenic cells from tissue culture into intraperitoneal and subcutaneous sites in nude mice. Tumor histopathology was assessed from hematoxylin and eosin stained sections from formalin-fixed paraffin-embedded (FFPE) tissues. Immunohistochemistry was carried out on FFPE tissues using cell type specific markers (CK7, FOXJ1, PAX8) to determine immunostaining patterns in mouse OCLER and FTLER xenografts as well as normal human ovaries and fallopian tubes online for full details of immunostaining procedure). Immunostaining was carried out using the conventional ABC technique. Metastasis of GFP-expressing tumor cells to lungs was analyzed initially using an Olympus SZX16 Stereo Fluorescence microscope in fresh tissues, followed by microscopic examination of hematoxylin and eosin stained sections of formalin-fixed, paraffin-embedded tissues. The presence of tumor cells in mouse lungs was confirmed by immunostaining for SV40 LT (Pab 101) and p53 (FL-393, Santa Cruz Biotechnology Inc).

8) RNA Extraction and Expression Profiling

Total RNA was extracted from each cell line in triplicate (different passages from the same cell line) using the RNeasy Mini kit (Qiagen, Valencia, Calif.) according to the manufacturer's instructions. RNA was checked with a size fractionation procedure using a capillary electrophoresis instrument (Bioanalyzer 2100, Agilent Technologies, Santa Clara, Calif.) to ensure high quality and RNA concentrations were estimated using the Nanodrop ND-1000 (NanoDrop Technologies Inc, Wilmington, Del.). Between 5-15 μg of RNA was used to generate biotinylated cDNA target that was subsequently hybridized to Affymetrix Human Genome U133 Plus 2.0 (Affymetrix Inc., Santa Clara, Calif.), according to standard protocols. All samples were profiled at the same time at the same microarray core facility to minimize batch effects.

9) Microarray Data Normalization and Analysis

Affymetrix microarrays of cell lines (OCE, FNE, OCLER, FNLER) and publically available ovarian cancer datasets by Wu et al. (GEO Series accession number GSE6008) and Tothill et al. (GEO Series accession number GSE9891) were independently normalized using the variance stabilization method (vsnrma) in R (version 2.10.1). Comparisons of gene expression between cell lines were performed using 24 Human Genome U133 Plus 2 microarrays (HG-U133Plus2, Affymetrix) measuring 54,675 probes. Samples that were arrayed included four biological replicates (immortalized and transformed cells from two patients) and three experimental replicates (different passages) for each cell type.

We first carried out unsupervised hierarchical clustering analysis based on global gene expression profiles and we observed strong separation between immortalized and transformed cells. Within each immortalized and transformed cluster the next subdivision of samples was by patient (1 or 2) and then by cell type (ovary or fallopian tube origin).

We applied a modified t-test (P<0.05) using Linear Models for Microarray Data (Limma) and correcting for the False Discovery Rate (FDR) to identify genes that were differentially expressed between cells of fallopian tube vs ovarian origin. These comparisons were carried out within immortalized and transformed cell groups as separate contrasts (e.g. FNE vs OCE; FNLER vs OCLER). Setting the FDR adjusted P-value at 0.05, 1,017 and 300 probes varied significantly between normal immortalized (FNE vs OCE) or transformed (FNLER vs OCLER) cells, respectively. To account for patient differences, we carried out a second analysis using Limma, this time using the duplicateCorrelation function to account for differences between patients. This confirmed a strong overlap between differentially expressed genes identified by the first and second (duplicateCorrelation) comparisons; thus we used the first set of gene signatures in subsequent analyses.

To classify human ovarian tumors as fallopian tube (FT)-like and ovary (OV)-like, genes were selected from the 1,017 probe (FNE vs OCE) signature (hereafter referred to as the ‘cell-of-origin’ signature) because we hypothesized that the carcinogenic transformation procedure could obscure inherent differences between fallopian tube and ovarian epithelial cells. To identify FT-like and OV-like subdivisions within two ovarian cancer datasets, we applied an approach that differs from previous widely used strategies of clustering patients based on global gene expression profiles. Instead we selected the five most highly significant probesets with unique gene symbols that were over-expressed in either FNE (DOK5, CD47, HS6ST3, DPP6, OSBPL3) or OCE cells (STC2, SFRP1, SLC35F3, SHMT2, TMEM164) and calculated the sum of the normalized expression values of these 10 genes in two ovarian cancer datasets by weighting FNE genes by (+1) and OCE genes by (−1); specifically, the sum of the normalized expression values of OCE genes were subtracted from the sum of expression values of FNE genes to calculate a score for each tumor (e.g. a higher score tumor is more FT-like). We then fit a bimodal distribution of Gaussian curves using mixture modeling to this score to identify two tumors within each dataset that were more OV-like vs FT-like.

We first performed this clustering in the Wu et al. dataset (GEO GSE6008) that contains expression profiles of 99 fresh frozen, microdissected epithelial ovarian cancers (including many non-serous histologic subtypes) arrayed on a similar platform (HG-U133A). Eight of the 10 selected genes were available for analysis due to array platform differences. We applied globaltests to determine the association between these eight genes and clinical factors in the Wu dataset (histologic subtype, grade and stage). We then applied linear logistic regression to identify specific genes that were most strongly associated with these clinical variables.

We used this 8-gene cell-of-origin signature to define FT-like and OV-like subpopulations in the Wu data (as discussed above) and visualized the distribution of these scores using density plots to determine the validity of this classification. We evaluated the clinical characteristics of the FT-like/OV-like classification and calculated their associated P-values using ordinal logistic regression (grade, stage) or Fisher's Exact Test (histologic subtype).

The 10-gene cell-of-origin signature was further validated in the Tothill dataset (GEO GSE9891); this includes 246 serous and 20 endometrioid fresh frozen malignant tumor pieces (not microdissected) that were arrayed on the same HG-U133Plus2 platform and could be linked to patient survival. The methods for Gaussian mixture modeling and tumor classification described for Wu were applied to the Tothill dataset. Further, to assess whether the FT-like/OV-like classification was associated with differences in patient disease-free and overall survival, we constructed Kaplan-Meier plots and applied a Cox proportional hazards test, adjusting for grade, stage and histologic subtype, to determine statistical significance. All microarray and survival analyses were conducted using R version 2.11.1.

10) Tumor Cell Culture

Fresh tumor tissue fragments were minced and plated on Primaria plates before and after digestion with 1 mg/ml collagenase. The tumor cells were cultured in an optimal nutrient medium (WIT-oc). The tumor cells were passaged at a ˜1:3 ratio once a week and plated into a new flask at approximately 1×10⁴ cells/cm². During the initial weeks of culture, the plates contain tumor cells as well as various normal epithelial and stromal cells. These non-tumor cells are more sensitive to trypsin and they come off the plate with 0.05% Trypsin treatment. Thus, during the early passages (˜1-5) the plates are treated with 0.05% trypsin first. The stromal and normal cells that come off the plate were removed with several washes with media. The remaining cells that are still attached to the culture plate were treated with 0.25% trypsin. In general tumor cultures were free of other cell types in 4-6 passages. The cells from papillary serous, clear cell, dysgerminoma, carcinosarcoma tumors were cultured in 5% CO₂ at 37° C. as monolayers attached to culture plates. The tumor cells from endometrioid and mucinous tumors were cultured in 5% CO₂ and 5% O₂ at 37° C. as monolayers attached to culture plates. The WIT-oc nutrient medium that was supplemented with 17 β-estrogen for endometrioid and mucinous tumors. The ATCC/ECACC cell lines were grown as per the instructions of the vendor.

11) Soft Agar Colony Assay

The cells from established cultures (passage 6-8) were harvested and plated in 0.4% agar. The well bottoms of a 12-well plate were sealed with 0.6% agar in prepared in WIT-oc medium to prevent monolayer formation. A single cell suspension in 0.4% agar in WIT-oc medium was added and allowed to set at room temperature, and placed in 37° C. incubators with 5% CO₂. The cells were fed with 0.4% agar in WIT-oc at 2 weeks, and colonies formation was observed after 2-4 weeks after plating.

Alternatively, tumor cells were grown in suspension cultures. The tumor spheres were grown in WIT-oc medium with 2% B27, 20 ng/ml EGF, 20 ng/ml bFGF (BD Biosciences), 4 ug/ml heparin, and 0.5% methyl cellulose. For sphere formation experiments, 15,000-20,000 cells/well were plated into 6-well ultra-low attachment plates (Corning), fed at days 1, 3, and 5, and spheres were counted at day 7.

12) Protein, DNA and RNA Analysis

The genomic DNA of tumor tissues were extracted from paraffin sections or when available from fresh tissues. The fresh tumor tissues were homogenized directly in RTL+ cell lysis buffer (Quiagen). The cell line lysates were prepared directly in the plate using the same lysis buffer. The DNA was extracted from the lysates using the Qiagen All-Prep mini kit. For Reverse-Phase Protein Analysis (RPPA) the semi-confluent cell monolayer cells were lysed in 125 ul RPPA lysis buffer on ice in triplicate (different passages from the same cell line). The RPPA extracts were probed with >200 antibodies. Total RNA was extracted from each cell line in triplicate (different passages from the same cell line) using the RNeasy Mini kit (Qiagen, Valencia, Calif.) according to the manufacturer's instructions. RNA was checked with a size fractionation procedure using a capillary electrophoresis instrument (Bioanalyzer 2100, Agilent Technologies, Santa Clara, Calif.) to ensure high quality and RNA concentrations were estimated using the Nanodrop ND-1000 (NanoDrop Technologies Inc, Wilmington, Del.). Between 5-15 μg of RNA was used to generate biotinylated cDNA target that was subsequently hybridized to Affymetrix Human Genome U133 Plus 2.0 (Affymetrix Inc., Santa Clara, Calif.), according to standard protocols. The genomic DNA from tumors and cell lines were analyzed with Affymetrix 250K Sty chips. Briefly, DNA is cleaved with Sty1, and the fragments are PCR amplified. The purified products are further fragmented with DNaseI, biotinylated, hybridized to a chip, and fluorescently labeled with phycoerythrin-conjugated streptavidin with signal amplification. SNP inferred LOH analysis was done using dCHIP and employed the hidden Markov mode.

13) Analysis of Tumorigenicity and Metastasis

Single-cell suspensions were prepared in a Matrigel: WIT mixture (1:1) and 1 million cells per 100 μl volume were injected in one intraperitoneal and two subcutaneous sites per mouse. Tumor cell injections were performed on 6-8 week old female immunodeficient nude (Nu/Nu) mice (Charles River Laboratories International, Inc, Wilmington, Mass.). Tumors were harvested 5 to 9 weeks after implantation of tumorigenic cells from tissue culture into subcutaneous and intraperitoneal sites in nude mice. Tumor histopathology was assessed from hematoxylin and eosin stained sections from formalin-fixed paraffin-embedded (FFPE) tissues.

14) Drug Sensitivity Experiments

The ATCC/ECACC ovarian tumor and the OCI ovarian tumor lines and were both plated in WIT-oc medium in 96 well plates in triplicate. The next day serial dilutions of MAPK inhibitor UO126 or microtubule inhibitor paclitaxel were added to the plate. The number of viable cells was measured as 590/530 florescence with an Alamar Blue assay.

### BIBLIOGRAPHY

1. Feeley, K. M. and M. Wells, Precursor lesions of ovarian epithelial malignancy. Histopathology, 2001. 38(2): p. 87-95.
2. Fox, H., Pathology of early malignant change in the ovary. Int J Gynecol Pathol, 1993. 12: p. 153-155.
3. Wong, A. S. and N. Auersperg, Ovarian surface epithelium: family history and early events in ovarian cancer. Reprod Biol Endocrinol, 2003. 1(1): p. 70.
4. Dubeau, L., The cell of origin of ovarian epithelial tumors and the ovarian surface epithelium dogma: does the emperor have no clothes? Gynecol Oncol, 1999. 72(3): p. 437-42.
5. Dubeau, L., The cell of origin of ovarian epithelial tumours. Lancet Oncol, 2008. 9(12): p. 1191-7.
6. Folkins, A. K., et al., A candidate precursor to pelvic serous cancer (p53 signature) and its prevalence in ovaries and fallopian tubes from women with BRCA mutations. Gynecol Oncol, 2008. 109(2): p. 168-73.
7. Jarboe, E. A., et al., Tubal and ovarian pathways to pelvic epithelial cancer: a pathological perspective. Histopathology, 2008. 53(2): p. 127-38.
8. Lee, Y., et al., A candidate precursor to serous carcinoma that originates in the distal fallopian tube. J Pathol, 2007. 211(1): p. 26-35.
9. Auersperg, N., S. L. Maines-Bandiera, and H. G. Dyck, Ovarian carcinogenesis and the biology of ovarian surface epithelium. J Cell Physiol, 1997. 173(2): p. 261-5.
10. Auersperg, N., et al., Characterization of cultured human ovarian surface epithelial cells: phenotypic plasticity and premalignant changes. Lab Invest, 1994. 71(4): p. 510-8.
11. Briton-Jones, C., et al., Human oviductin mRNA expression is not maintained in oviduct mucosal cell culture. Fertil Steril, 2002. 77(3): p. 576-80.
12. Corner, M. T., H. J. Leese, and J. Southgate, Induction of a differentiated ciliated cell phenotype in primary cultures of Fallopian tube epithelium. Hum Reprod, 1998. 13(11): p. 3114-20.
13. Okamura, H., H. Katabuchi, and T. Ohba, What we have learned from isolated cells from human ovary? Mol Cell Endocrinol, 2003. 202(1-2): p. 37-45.
14. Gregoire, L., et al., Organotypic culture of human ovarian surface epithelial cells: a potential model for ovarian carcinogenesis. In Vitro Cell Dev Biol Anim, 1998. 34(8): p. 636-9.
15. Liu, J., et al., A genetically defined model for human ovarian cancer. Cancer Res, 2004. 64(5): p. 165563.
16. Ince, T. A., et al., Transformation of Different Human Breast Epithelial Cell Types Leads to Distinct Tumor Phenotypes. Cancer Cell, 2007. 12(2): p. 160-170.
17. Elenbaas, B., et al., Human breast cancer cells generated by oncogenic transformation of primary mammary epithelial cells. Genes Dev, 2001. 15(1): p. 50-65.
18. Cho, K R., Ovarian cancer update: lessons from morphology, molecules, and mice. Arch Pathol Lab Med, 2009. 133(11): p. 1775-81.
19. Wu, R., et al., Mouse model of human ovarian endometrioid adenocarcinoma based on somatic defects in the Wnt/beta-catenin and PI3K/Pten signaling pathways. Cancer Cell, 2007. 11(4): p. 321-33.
20. Huber, W., et al., Variance stabilization applied to microarray data calibration and to the quantification of differential expression. Bioinformatics, 2002. 18 Suppl 1: p. S96-104.
21. Smyth, G. K., Linear models and empirical bayes methods for assessing differential expression in microarray experiments. Stat Appl Genet Mol Biol, 2004. 3: p. Article 3.
22. Monti, S., et al., Consensus Clustering: A Resampling-Based Method for Class Discovery and Visualization of Gene Expression Microarray Data. Machine Learning, 2003. 52: p. 91-118.
23. Verhaak, R. G., et al., Integrated genomic analysis identifies clinically relevant subtypes of glioblastoma characterized by abnormalities in PDGFRA, IDH1, EGFR, and NF1. Cancer Cell, 2010. 17(1): p. 98
24. Verschraegen C F, et. al. Establishment and characterization of cancer cell cultures and xenografts derived from primary or metastatic Mullerian cancers. (Clin Cancer Res. 2003 February; 9(2):845-52.
25. Baba A I, Câtoi C., Comparative Oncology. Bucharest: The Publishing House of the Romanian Academy; 2007

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A cell culture medium, or a kit for preparing a cell culture medium, comprising:
(a) adenosine triphosphate;
(b) a carrier protein;
(c) cholesterol, linoleic acid, and lipoic acid;
(d) glutathione;
(e) a nucleotide salvage pathway precursor base selected from hypoxanthine, xanthine, adenine, guanine and thymidine;
(f) phosphoethanolamine;
(g) selenium;
(h) transferrin;
(i) triiodothyronine;
vitamin A, vitamin C, and vitamin D;
(k) Zn, Mg, and Cu;
(l) an agent that increases intracellular cAMP;
(m) epidermal growth factor (EGF);
(n) hydrocortisone;
(o) insulin; and
(p) serum.

2. The cell culture medium of claim 1, further comprising one or more of:
(a) adenosine monophosphate;
(b) vitamin E; or
(c) at least one of vitamin K3, niacin, or niacinamide.

3. (canceled)

4. The cell culture medium of claim 1, wherein the carrier protein is albumin.

5-7. (canceled)

8. The cell culture medium of claim 1, wherein the agent that increases intracellular cAMP is cholera toxin.

9-24. (canceled)

25. The cell culture medium of claim 1, further comprising an estrogen.

26-27. (canceled)

28. The cell culture medium of claim 25, wherein the estrogen is 17-beta-estradiol.

29. The cell culture medium of claim 1, wherein the medium is substantially free of estrogen.

30-36. (canceled)

37. The cell culture medium of claim 1, wherein the medium supports proliferation of ovarian tumor cells for at least about 15 population doublings (PD) in vitro.

38. The cell culture medium of claim 1, wherein the medium supports proliferation of ovarian cells and/or fallopian tube cells for at least about 15 population doublings (PD) in vitro.

39. A kit for preparing the cell culture medium of claim 1, comprising a first one or more containers comprising components (a)-(k) and a second one or more containers comprising components (l)-(p) and optionally estrogen, whereby combining the contents of the first and second containers in an appropriate proportion results in the cell culture medium.

40. A cell culture medium supplement, wherein the supplement comprises:
(a) an agent that increases intracellular cAMP;
(b) epidermal growth factor (EGF);
(c) hydrocortisone;
(d) insulin;
(e) serum; and optionally,
(f) an estrogen,
wherein adding the supplement to a basal cell culture medium results in the cell culture medium of claim 1.

41. A method of preparing the cell culture medium of claim 1, comprising combining components (a)-(p) and optionally estrogen.

42. (canceled)

43. A method for culturing ovarian and fallopian tube cells, comprising:
(a) obtaining ovarian and/or fallopian tube cells from an ovary or a fallopian tube;
(b) culturing the cells in the cell culture medium of claim 1,
wherein the cell culture medium supports at least 15 population doublings of the ovarian and fallopian tube epithelial cells.

44. A method for culturing tumor cells, comprising:
(a) obtaining tumor cells;
(b) culturing the tumor cells in the cell culture medium of claim 1,
wherein the cell culture medium supports at least 15 population doublings of the tumor cells.

45-51. (canceled)

52. A composition comprising
the cell culture medium of claim 1, and
cells, wherein the cells are immortalized ovarian cells and/or fallopian tube cells.

53-58. (canceled)

59. A method of identifying candidate therapeutic agents, comprising:
(a) culturing cells according to the method of claim 43;
(b) contacting the cells with an agent; and
(c) measuring one or more physiological features of the cells;
wherein the agent that modulates the one or more physiological features of the cells is a candidate therapeutic agent.

60-65. (canceled)

66. A substantially purified culture of ovarian cells, wherein the ovarian cells overexpress the probesets DOK5, CD47, HS6ST3, DPP6, OSBLP3; wherein the culture comprises at least 10³ cells; and wherein the cells are capable of undergoing at least 14 population doublings.

67. A substantially purified culture of fallopian tube cells, wherein the fallopian tube cells overexpress the probesets STC2, SFRP1, SLC35F3, SHMT2, TMEM164; wherein the culture comprises at least 10³ cells, and wherein the cells are capable of undergoing at least 14 population doublings.

68-70. (canceled)

71. A method of identifying candidate therapeutic agents, comprising:
(a) culturing cells according to the method of claim 44;
(b) contacting the cells with an agent; and
(c) measuring one or more physiological features of the cells;
wherein the agent that modulates the one or more physiological features of the cells is a candidate therapeutic agent.
